(19) United States
(12) Patent Application Publication
(10) Pub. No.: US 2025/0326739 A1
(43) Pub. Date: Oct. 23, 2025

(54) CD38 MODULATORS AND USES THEREOF

(71) Applicant: Aeovian Pharmaceuticals, Inc., Berkeley, CA (US)

(72) Inventor: John KINCAID, Fox Point, WI (US)

(21) Appl. No.: 18/960,209

(22) Filed: Nov. 26, 2024

Related U.S. Application Data
(63) Continuation of application No. PCT/US23/67887, filed on Jun. 2, 2023.
(60) Provisional application No. 63/348,381, filed on Jun. 2, 2022.
(60) Provisional application No. 63/376,647, filed on Sep. 22, 2022.

(51) Int. Cl. C07D 401/14; A61K 31/4439; A61K 31/4545; A61K 31/506; A61K 31/5377; C07D 401/04; C07D 405/14; C07D 409/14; C07D 493/08

(52) U.S. Cl. CPC C07D 401/14; A61K 31/4439; A61K 31/4545; A61K 31/506; A61K 31/5377; C07D 401/04; C07D 405/14; C07D 409/14; C07D 493/08

(57) ABSTRACT

The disclosure provides compounds and salts for the treatment of disease, such as Formula (I), Formula (I-A), Formula (I-B), Formula (I-C), or Formula (II).

### CROSS-REFERENCE

This application is a continuation of International Patent Application No. PCT/US2023/067887 filed on Jun. 2, 2023, which claims the benefit of U.S. Provisional Patent Applications Nos. 63/348,381 filed on Jun. 2, 2022; and 63/376,647 filed on Sep. 22, 2022; each of each of which is incorporated herein by reference in its entirety.

### BACKGROUND OF THE INVENTION

Nicotinamide Adenine Dinucleotide (NAD⁺) is a biochemical found in all cells that was first characterized over 100 years ago due to its role in oxidoreductase reactions. Since then, NAD⁺ and its related pyridine nucleotides NADH, NADP⁺, and NADPH are recognized as the major redox carriers in all organisms. These pyridine dinucleotides regulate the cytosolic and mitochondrial redox state and are key participants monitoring the metabolic status of the cell. This is because NAD⁺ and NADH act as hydride accepting and donating cofactors for metabolic enzymes involved in glycolysis, the TCA cycle, and the respiratory chain and thereby redistribute reducing equivalents generated from these catabolic processes into the de novo synthesis of new biomolecules. (Houtkooper et al Endo Reviews (2010) 31:194-223; Koch-Nolte et al Science Signaling (2009) 2:mr1; Houtkooper and Auwerx J. Cell Biol (2012) 199:205-209; Berger et al Trends in Bioch Sci (2004) 29:111-18).

In addition to its long recognized role as a cofactor for oxidoreductases, more recent research demonstrates that NAD⁺ is also a substrate for various enzymes, where it is consumed in the process of donating its ADP ribose to acceptor molecules. The enzymes that are the major consumers of NAD+ are the ADP ribosyl transferases (i.e., PARP and ART family of enzymes), the sirtuins (Sirt1-7), and the ADP ribosyl cyclases/hydrolases (CD38/CD157). These enzymes are involved in pathways that regulate Ca⁺⁺ signaling, gene transcription, DNA repair, cell survival, energy metabolism, and oxidative stress. Thus, NAD⁺ and its phosphorylated relatives NADP and NAADP, both of which are derived from NAD⁺, also act as signaling molecules. NAD⁺ is also a key component of the circadian cycle with daily oscillations that tie cellular metabolism to chromatin remodeling and gene transcription.

It is known that exercise and caloric restriction elevate NAD⁺ levels, while aging and obesity decrease cellular NAD⁺ levels. Restoring NAD⁺ levels in disease states that consume significant amounts of NAD⁺ will likely have medical benefits as the cell strives to maintain its energy status during stress. (Tevy et al Trends in Endo and Metab (2013) 24:229-237; Pugh et al Aging Cell (2013) 12:672-681; Massudi et al PLoS ONE (2012) 7:e42357; Xu and Sauve (2010) Mech of Ageing and Development 131:287-298). Cellular NAD⁺ is produced by either the de novo synthesis pathway from tryptophan or by a salvage synthesis pathway from precursors such as nicotinic acid (niacin) and nicotinamide, both of which are obtained from dietary sources.

A third way to modulate cellular NAD⁺ levels is to block consumption of NAD⁺ by inhibiting enzymes that consume NAD⁺. CD38 is one such consumer of NAD. Also known as ADP ribosyl cyclase, CD38 is a type II membrane-anchored enzyme. It efficiently catalyzes the breakdown of NAD⁺ to nicotinamide and ADPR and hydrolyzes NAADP to ADPRP. CD38 can also act as a cyclase converting NAD⁺ to cADPR, although it is 100-fold less efficient as a cyclase than as a hydrolase.

CD38 was first characterized as a surface antigen on immune cells and is broadly distributed throughout most tissues in the body. It exists on the plasma membrane and on the membranes of intracellular organelles such as the nucleus and mitochondria. As predicted from its function as a NAD⁺ glycohydrolase, CD38 KO mice have elevated NAD⁺ levels relative to wild-type controls. Likewise, inhibitors of CD38 enzyme activity also modulate NAD⁺ tissue levels and would be useful in treating various diseases where CD38 is over expressed or where cellular NAD⁺ levels are depressed or desynchronized. Compounds which inhibit CD38 and thereby raise NAD⁺ levels are useful in treating diseases or conditions indicated to benefit from NAD⁺ including mitochondrial-related diseases or disorders.

### SUMMARY OF THE INVENTION

In an aspect, the present disclosure provides a compound represented by the structure of Formula I:

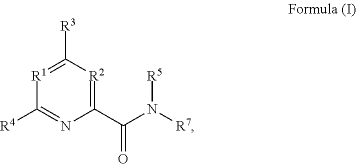

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R⁷ is selected from an optionally substituted saturated 4- to 8-membered heterocycle, optionally substituted C₃₋₅ cycloalkyl, optionally substituted C₇₋₁₀ cycloalkyl, and substituted C₆ cycloalkyl, wherein the 4- to 8-membered heterocycle, C₃₋₅ cycloalkyl, and C₇₋₁₀ cycloalkyl are each optionally substituted with one or more R⁸, and the C₆ cycloalkyl is substituted with one or more R^{8A}, and the C₆ cycloalkyl is further optionally substituted with one or more R^{8B};
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle; wherein when R⁴ is imidazole, R³ is hydrogen, R⁷ is pyrrolidine substituted with oxo and haloalkyl, the haloalkyl is selected from C₁ haloalkyl and C₃₋₆ haloalkyl; and wherein when R¹ is N, R³ is imidazole, R⁷ is C₄ cycloalkyl, and R⁸ is —OR²⁰, the C₁₋₆ alkyl of R²⁰ is selected from C₁ alkyl and C₃₋₆ alkyl;
R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂;
R^{8B} is independently selected each occurrence from —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, and C₂₋₆ alkynyl;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In an aspect, the present disclosure provides a compound of Formula (I-B):

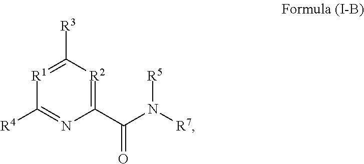

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R⁷ is selected from:
   a saturated 4- to 8-membered heterocycle, wherein the saturated 4- to 8-membered heterocycle is substituted with at least one substitutent selected from —S(O)₂(R²⁰), —S(O)(R²⁰), —S(O)₂(NR²⁰²), —S(O)(NR²¹)R²⁰, and —S(O)(NR²¹)N(R²¹)₂; and wherein the saturated 4- to 8-membered heterocycle is further optionally substituted with one or more R⁸;
   a C₃₋₅ cycloalkyl, and C₇₋₁₀ cycloalkyl, each of which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein each is further optionally substituted with one or more R⁸;
   a C₆ cycloalkyl substituted with at least one substitutent selected from —S(O)₂(R²⁰), —S(O)(R²⁰), —S(O)₂(NR²⁰₂), —S(O)(NR²¹)R²⁰; and wherein the C₆ cycloalkyl is further optionally substituted with one or more R^{BA};
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In an aspect, the present disclosure provides a compound of Formula (I-A):

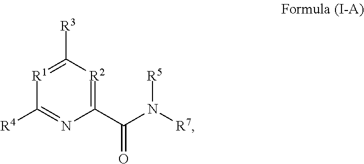

or a pharmaceutically acceptable salt thereof, wherein:
R¹ is selected from N and CR¹¹;
R² is selected from N and CR¹²;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R³ is selected from halogen, —O—C₁-C₆ alkyl, and C₁-C₆ alkyl, wherein the C₁-C₆ alkyl is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl; and
R⁷ is selected from an optionally substituted saturated 4- to 8-membered heterocycle, which is optionally substituted with one or more R⁸;
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In an aspect, the present disclosure provides a compound of Formula (II):

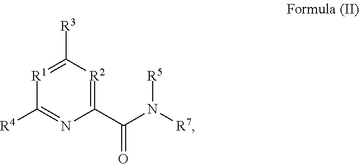

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is imidazole, wherein the imidazole is optionally substituted with one or more R⁹;
R⁵ is selected from hydrogen and C₁-C₆ alkyl;
R⁷ is selected from an 4- to 8-membered heterocycle and C₃₋₁₂ carbocycle, each of which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the 4- to 8-membered heterocycle and C₃₋₁₂ carbocycle are each further optionally substituted with one or more R⁸;
each R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In certain embodiments, the disclosure provides a pharmaceutical composition comprising a compound or salt of Formula (I), Formula (I-A), Formula (I-B), Formula (I-C), or Formula (II), and a pharmaceutically acceptable excipient.

In certain embodiments, the disclosure provides a method of treating a disease, comprising administering to a subject in need thereof a compound or salt of Formula (I), Formula (I-A), Formula (I-B), Formula (I-C), or Formula (II), or a pharmaceutical composition comprising a compound or salt of Formula (I), Formula (I-A), Formula (I-B), Formula (I-C), or Formula (II), and a pharmaceutically acceptable excipient. In some cases, the subject would benefit from inhibition of CD38. In some cases, the disease is a neurodegenerative disease.

In certain embodiments, the disclosure provides a method of inhbiting CD38 comprising administering a compound or salt of Formula (I), Formula (I-A), Formula (I-B), Formula (I-C), or Formula (II). In some cases, the method comprises inhibiting CD38 by administering a pharmaceutical composition comprising a compound or salt of Formula (I), Formula (I-A), Formula (I-B), Formula (I-C), or Formula (II), and a pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to saturated hydrocarbon groups, including straight-chain alkyl and branched-chain alkyl groups that contain from 1 to 6 carbons. The term —C_{x-y} alkylene-refers to a substituted or unsubstituted alkylene chain with from x to y carbons in the alkylene chain. For example —C₁₋₆ alkylene—may be selected from methylene, ethylene, propylene, butylene, pentylene, and hexylene, any one of which is optionally substituted.

“Alkyl” as used herein refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkenyl” as used herein refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” as used herein refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y} alkenyl” and “C_{x-y} alkynyl” refer to unsaturated aliphatic groups analogous in length and possible substitution to the alkyls described above, but that contain at least one double or triple bond, respectively. The term —C_{x-y} alkenylene—refers to a substituted or unsubstituted alkenylene chain with from x to y carbons in the alkenylene chain. For example, —C₂₋₆ alkenylene—may be selected from ethenylene, propenylene, butenylene, pentenylene, and hexenylene, any one of which is optionally substituted. An alkenylene chain may have one double bond or more than one double bond in the alkenylene chain. The term —C_{x-y} alkynylene-refers to a substituted or unsubstituted alkynylene chain with from x to y carbons in the alkynylene chain. For example, —C₂₋₆ alkynylene—may be selected from ethynylene, propynylene, butynylene, pentynylene, and hexynylene, any one of which is optionally substituted. An alkynylene chain may have one triple bond or more than one triple bond in the alkynylene chain.

“Alkylene” refers to a straight divalent hydrocarbon chain linking the rest of the molecule to a radical group, consisting solely of carbon and hydrogen, containing no unsaturation, and preferably having from one to twelve carbon atoms, for example, methylene, ethylene, propylene, butylene, and the like. The alkylene chain is attached to the rest of the molecule through a single bond and to the radical group through a single bond. The points of attachment of the alkylene chain to the rest of the molecule and to the radical group are through the terminal carbons respectively. An alkylene chain may be optionally substituted by one or more substituents such as those substituents described herein.

“Alkenylene” refers to a straight divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group are through the terminal carbons respectively. An alkenylene chain may be optionally substituted by one or more substituents such as those substituents described herein.

“Alkynylene” refers to a straight divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group are through the terminal carbons respectively. An alkynylene chain may be optionally substituted by one or more substituents such as those substituents described herein.

“Halo” or “halogen” as used herein refers to halogen substituents such as bromo, chloro, fluoro and iodo substituents.

“Haloalkyl” as used herein refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, and any other suitable combinations of alkanes (or substituted alkanes) and halogens. When an alkyl group is substituted with more than one halogen radicals, each halogen may be independently selected, for example 1-chloro,2-bromoethane.

“Aminoalkyl” refers to an alkyl radical, as defined above, that is substituted by one or more amine radicals, for example, propan-2-amine, butane-1,2-diamine, pentane-1,2,4-triamine and the like.

“Hydroxyalkyl” refers to an alkyl radical, as defined above, that is substituted by one or more hydroxy radicals, for example, propan-1-ol, butane-1,4-diol, pentane-1,2,4-triol, and the like.

“Alkoxyalkyl” refers to an alkyl radical, as defined above, that is substituted by one or more alkoxy radicals, for example, methoxymethane, 1,3-dimethoxybutane, 1-methoxypropane, 2-ethoxypentane, and the like.

“Cyanoalkyl” as used herein refers to an alkyl radical, as defined above, that is substituted by one or more cyano radicals, for example, acetonitrile, 2-ethyl-3-methylsuccinonitrile, butyronitrile, and the like.

The term “carbocycle” as used herein refers to a saturated, unsaturated or aromatic ring in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Bicyclic carbocycles may be fused, bridged or spiro-ring systems. A carbocycle may be optionally substituted by one or more substituents such as those substituents described herein.

The term “unsaturated carbocycle” refers to carbocycles with at least one degree of unsaturation and excluding aromatic carbocycles. Examples of unsaturated carbocycles include cyclohexadiene, cyclohexene, and cyclopentene.

The term “cycloalkyl” as used herein refers to a saturated carbocycle. Exemplary cycloalkyl rings include cyclopropyl, cyclohexyl, and norbornane. Cycloalkyls may be optionally substituted by one or more substituents such as those substituents described herein.

The term “C_{x-y} carbocycle” is meant to include groups that contain from x to y carbons in the cycle. For example, the term “C₃₋₆ carbocycle” refers to a saturated, unsaturated, or aromatic ring comprising from 3 to 6 carbons. For example —C₃₋₆ carbocycle—may be selected from cyclopropyl, cyclobutyl, cyclopentyl, cyclohexyl, and phenyl, any one of which is optionally substituted.

“Aryl” as used herein refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hickel theory. The ring system from which aryl groups are derived include, but are not limited to, groups such as benzene, fluorene, indane, indene, tetralin and naphthalene.

The term “heterocycle” as used herein refers to a saturated, unsaturated or aromatic ring comprising one or more heteroatoms. Exemplary heteroatoms include N, O, Si, P, B, and S atoms. The heterocycle may be attached to the rest of the molecule through any atom of the heterocycle, valence permitting, such as a carbon or nitrogen atom of the heterocycle. Heterocycles include 3- to 10-membered monocyclic rings, 6- to 12-membered bicyclic rings, and 6- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Bicyclic heterocycles may be fused, bridged, or spiro-ring systems. A spiro-ring system may be referred as a “spiro heterocycle” or “spiroheterocycle” or “spiro-ring heterocycle”. In some cases, spiro heterocycle, spiro-ring heterocycles or spiroheterocycle have at least two molecular rings with only one common atom. The spiro heterocycle, spiro-ring heterocycle or spiroheterocycle includes one or more heteroatoms.

“Heteroaryl” or “aromatic heterocycle” refers to a radical derived from a heteroaromatic ring radical that comprises one to eleven carbon atoms and at least one heteroatom wherein each heteroatom may be selected from N, O, and S. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hickel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine.

An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc.

The term “unsaturated heterocycle” refers to heterocycles with at least one degree of unsaturation and excluding aromatic heterocycles. Examples of unsaturated heterocycles include dihydropyrrole, dihydrofuran, oxazoline, pyrazoline, and dihydropyridine. Heterocycles may be optionally substituted by one or more substituents such as those substituents described herein.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—OR^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—OR^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—OR^{a}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain. It will be understood by those skilled in the art that substituents can themselves be substituted, if appropriate.

As used herein, the term “optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “subject,” “individual,” and “patient” may be used interchangeably and refer to humans, the as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for, a pathology to be prophylactically or therapeutically treated with a compound or salt described herein.

The terms “administer”, “administered”, “administers” and “administering” are defined as providing a composition to a subject via a route known in the art, including but not limited to intravenous, intraarterial, oral, parenteral, buccal, topical, transdermal, rectal, intramuscular, subcutaneous, intraosseous, transmucosal, or intraperitoneal routes of administration. In certain embodiments, oral routes of administering a composition can be used. The terms ““administer”, “administered”, “administers” and “administering” a compound should be understood to mean providing a compound of the invention or a prodrug of a compound of the invention to the individual in need.

The term “effective amount” or “therapeutically effective amount” refers to that amount of a compound or salt described herein that is sufficient to effect the intended application including but not limited to disease treatment, as defined below. The therapeutically effective amount may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term can also apply to a dose that can induce a particular response in target cells, e.g., reduction of proliferation or down regulation of activity of a target protein. The specific dose can vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including, but not limited to, a therapeutic benefit and/or a prophylactic benefit. In certain embodiments, treatment or treating involves administering a compound or composition disclosed herein to a subject. A therapeutic benefit may include the eradication or amelioration of the underlying disorder being treated. Also, a therapeutic benefit may be achieved with the eradication or amelioration of one or more of the physiological symptoms associated with the underlying disorder, such as observing an improvement in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. Treating can be used herein to refer to a method that results in some level of treatment or amelioration of the disease or condition, and can contemplate a range of results directed to that end, including but not restricted to prevention of the condition entirely.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “selective inhibition” or “selectively inhibit” as referred to a biologically active agent refers to the agent's ability to preferentially reduce the target signaling activity as compared to off-target signaling activity, via direct or interact interaction with the target.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The section headings used herein are for organizational purposes only and are not to be construed as limiting the subject matter described.

### Compounds

In an aspect, the present disclosure provides a compound represented by the structure of Formula I:

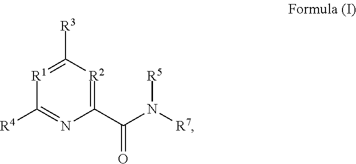

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R⁷ is selected from an optionally substituted saturated 4- to 8-membered heterocycle, optionally substituted C₃₋₅ cycloalkyl, optionally substituted C₇₋₁₀ cycloalkyl, and substituted C₆ cycloalkyl, wherein the 4- to 8-membered heterocycle, C₃₋₅ cycloalkyl, and C₇₋₁₀ cycloalkyl are each optionally substituted with one or more R⁸, and the C₆ cycloalkyl is substituted with one or more R^{8A}, and the C₆ cycloalkyl is further optionally substituted with one or more R^{8B};
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle; wherein when R⁴ is imidazole, R³ is hydrogen, R⁷ is pyrrolidine substituted with oxo and haloalkyl, the haloalkyl is selected from C₁ haloalkyl and C₃₋₆ haloalkyl; and wherein when R¹ is N, R³ is imidazole, R⁷ is C₄ cycloalkyl, and R⁸ is —OR²⁰, the C₁₋₆ alkyl of R²⁰ is selected from C₁ alkyl and C₃₋₆ alkyl;
R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂;
R^{8B} is independently selected each occurrence from —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkyl, C₂₋₆ alkenyl, and C₂₋₆ alkynyl;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, Formula (I) is represented by Formula (I-A)

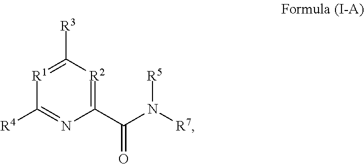

or a pharmaceutically acceptable salt thereof, wherein:
R³ is selected from halogen, —O—C₁-C₆ alkyl, and C₁-C₆ alkyl, wherein the C₁-C₆ alkyl is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl; and
R⁷ is selected from an optionally substituted saturated 4- to 8-membered heterocycle, which is optionally substituted with one or more R⁸.

In some embodiments, Formula (I) is represented by Formula (I-B):

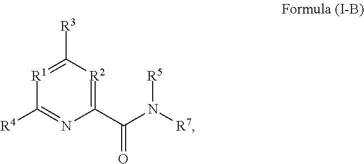

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R⁷ is selected from:
   a saturated 4- to 8-membered heterocycle, wherein the saturated 4- to 8-membered heterocycle is substituted with at least one substitutent selected from —S(O)₂(R²⁰), —S(O)(R²⁰), —S(O)₂(NR²⁰²), —S(O)(NR²¹)R²⁰, and —S(O)(NR²¹)N(R²¹)₂; and wherein the saturated 4- to 8-membered heterocycle is further optionally substituted with one or more R⁸;
   a C₃₋₅ cycloalkyl, and C₇₋₁₀ cycloalkyl, each of which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein each is further optionally substituted with one or more R⁸;
   a C₆ cycloalkyl substituted with at least one substitutent selected from —S(O)₂(R²⁰), —S(O)(R²⁰), —S(O)₂(NR²⁰₂), —S(O)(NR²¹)R²⁰; and wherein the C₆ cycloalkyl is further optionally substituted with one or more R^{8A};
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), S(O)₂(NR²⁰₂), —S(O)(NR²¹)R²⁰, and —S(O)(NR²¹)N(R²¹)₂;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (I), is represented by Formula (I-C)

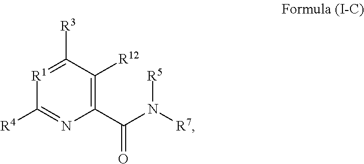

or a pharmaceutically acceptable salt thereof, wherein:
R¹² is selected from halogen, —NH₂, C₁-C₆ alkyl, —CF₃, and —O—C₁₋₁₀ alkyl; and
R⁷ is selected from an optionally substituted saturated 4- to 8-membered heterocycle, which is optionally substituted with one or more R⁸.

In some embodiments, for a compound or salt of Formula (I), Formula (I-A), or Formula (I-B), R³ is selected from halogen, —CN, —OR¹⁶, C₁-C₆ alkyl, C₃₋₆ carbocycle, and 3- to 6-membered heterocycle, wherein the C₁-C₆ alkyl, C₃₋₆ carbocycle, and 3- to 6-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, and C₁-C₆ alkyl. In some cases, R³ is selected from —O—C₁-C₆ alkyl and C₁-C₆ alkyl, wherein the C₁-C₆ alkyl is optionally substituted with one or more substituents independently selected from halogen, —OH, —CN, —NH₂, —NHC₁₋₁₀ alkyl, —N(C₁₋₁₀ alkyl)₂, —C₁₋₁₀ haloalkyl, and —O—C₁₋₁₀ alkyl. In some cases, R³ is selected from C₁-C₆ alkyl, wherein the C₁-C₆ alkyl is optionally substituted with one or more substituents independently selected from halogen, —OH, —CN, —NH₂, —NHC₁₋₁₀ alkyl, —N(C₁₋₁₀ alkyl)₂, —C₁₋₁₀ haloalkyl, and —O—C₁₋₁₀ alkyl. In some cases, R³ is selected from C₁-C₆ alkyl. In some cases, R³ is selected from methyl.

In some embodiments, for a compound or salt of Formula (I), Formula (I-A), or Formula (I-B), R¹ is selected from CR¹¹. In some cases, R¹ is N. In some cases, R² is selected from CR¹¹. In some cases, R² is N. In some cases, R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹¹ is selected from hydrogen, halogen, —OH, —CN, —O—C₁₋₁₀ alkyl, C₁-C₆ alkyl, and C₁-C₆ haloalkyl. In some cases, R¹¹ is selected from hydrogen, halogen, C₁-C₆ alkyl, and C₁-C₆ haloalkyl. In some cases, R¹¹ is selected from hydrogen, and C₁-C₆ alkyl. In some cases, R¹¹ is hydrogen. In some cases, R¹ is selected from N and CH. In some cases, R¹ is CH.

In some embodiments, for a compound or salt of Formula (I), Formula (I-A), or Formula (I-B), R² is selected from CR¹². In some cases, R² is N. In some cases, R² is selected from CR¹². In some cases, R² is N. In some cases, R² is selected from CR¹². In some cases, R² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹² is selected from hydrogen, halogen, —OH, —CN, —O—C₁₋₁₀ alkyl, C₁-C₆ alkyl, and C₁-C₆ haloalkyl. In some cases, R¹² is selected from hydrogen, halogen, C₁-C₆ alkyl, and C₁-C₆ haloalkyl. In some cases, R¹² is selected from hydrogen, and C₁-C₆ alkyl. In some cases, R¹² is hydrogen. In some cases, R² is selected from N and CH. In some cases, R² is CH.

In some embodiments, for a compound or salt of Formula (I), Formula (I-A), or Formula (I-B), R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, and C₁-C₆ alkyl. In some cases, R³ is selected from hydrogen, —OR¹⁶, optionally substituted C₁-C₆ alkyl, and 3- to 12-membered heterocycle, wherein the C₁-C₆ alkyl and 3- to 12-membered heterocycle are each optionally substituted with one or more substituents independently selected from halogen, —OH, —CN, —NO₂, —NH₂, —NHC₁₋₁₀ alkyl, C₁-C₆ alkyl, —N(C₁₋₁₀ alkyl)₂, —C₁₋₁₀ haloalkyl, and —O—C₁₋₁₀ alkyl. In some cases, R³ is selected from hydrogen, —OR¹⁶, C₁-C₆ alkyl, and 5- to 6-membered heterocycle, wherein the 5- to 6-membered heterocycle is optionally substituted with one or more substituents independently selected from C₁-C₆ alkyl. In some cases, R³ is selected from hydrogen, halogen, —CN, —OR¹⁶, and C₁-C₆ alkyl. In some cases, R³ is selected from hydrogen, halogen, —CN, and —OR¹⁶. In some cases, R³ is selected from hydrogen, halogen, and —CN. In some cases, R³ is selected from hydrogen and halogen. In some cases, R³ is selected from hydrogen and C₁-C₆ alkyl. In some cases, R³ is selected from C₁-C₆ alkyl. In some cases, R³ is methyl. In some cases, R³ is selected from halogen. In some cases, R³ is —SO₂R¹⁶.

In some embodiments, for a compound or salt of Formula (I), Formula (I-A), or Formula (I-B), R³ is selected from C₁-C₆ alkyl optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some embodiments, R³ is selected from C₁-C₆ alkyl optionally substituted with one or more substituents independently selected from halogen, —OH, —CN, —NO₂, and —NH₂. In some embodiments, R³ is selected from C₁-C₆ alkyl optionally substituted with one or more substituents independently selected from halogen, —OH, and —CN. In some embodiments, R³ is selected from methyl and ethyl. In some embodiments, R³ is methyl.

In some embodiments, for a compound or salt of Formula (I), Formula (I-A), or Formula (I-B), R³ is selected from halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle. In some cases, R³ is selected from halogen, —CN, —OR¹⁶, —SO₂R¹⁶, C₁-C₆ alkyl, C₃₋₁₂ carbocycle, and 3- to 12-membered heterocycle. In some cases, R³ is selected from C₁-C₆ alkyl and —OR¹⁶.

In some embodiments, for a compound or salt of Formula (I), Formula (I-A), or Formula (I-B), R³ is —OR¹⁶. In some cases, R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₆ carbocycle, wherein the C₁₋₆ alkyl, and C₃₋₆ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from hydrogen; C₁₋₆ alkyl, and C₃₋₁₂ carbocycle. In some cases, R¹⁶ is selected from C₁₋₆ alkyl and C₃₋₆ carbocycle. In some cases, R¹⁶ is selected from methyl and phenyl. In some cases, R¹⁶ is selected from C₁₋₆ alkyl, wherein the C₁₋₆ alkyl is optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl.

In some embodiments, for a compound or salt of Formula (I), Formula (I-A), or Formula (I-B), R³ is —SO₂R¹⁶. In some cases, R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₆ carbocycle, wherein the C₁₋₆ alkyl, and C₃₋₆ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from hydrogen; C₁₋₆ alkyl, and C₃₋₁₂ carbocycle. In some cases, R¹⁶ is selected from C₁₋₆ alkyl and C₃₋₆ carbocycle. In some cases, R¹⁶ is selected from methyl and phenyl. In some cases, R¹⁶ is selected from C₁₋₆ alkyl, wherein the C₁₋₆ alkyl is optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl.

In some embodiments, for a compound or salt of Formula (I), Formula (I-A), or Formula (I-B), R³ is selected from a 3- to 12-membered heterocycle, wherein the 3- to 12-membered heterocycle is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle. In some cases, R³ is selected from a 5- to 6-membered heterocycle, which is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl. In some cases, R³ is selected from a 5- to 6-membered heterocycle, which is optionally substituted with one or more substituents independently selected from C₁-C₆ alkyl. In some cases, R³ is selected from a 5-membered heterocycle, which is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl. In some cases, R³ is selected from a 6-membered heterocycle, which is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl. In some cases, R³ is selected from a 5-membered heterocycle, which is optionally substituted with one or more substituents independently selected from C₁-C₆ alkyl. In some cases, the heterocycle for R³ contains at least 1 nitrogen atom. In some cases, the heterocycle for R³ contains at least 2 nitrogen atoms. In some cases, R³ is

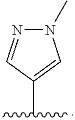 [SMILES: *c1cnn(C)c1]

In some embodiments, for a compound or salt of Formula (I), Formula (I-A), or Formula (I-B), R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is unsubstituted. In some cases, R⁴ is selected from a 5- to 6-membered heteroaryl, wherein the 5- to 6-membered heteroaryl is selected from imidazole, and thiazole, each of which is unsubstituted. In some cases, R⁴ is selected from a 5- to 6-membered heteroaryl, wherein the 5- to 6-membered heteroaryl is selected from unsubstituted imidazole. In some cases, R⁴ is selected from a 5- to 6-membered heteroaryl, wherein the 5- to 6-membered heteroaryl is selected from unsubstituted thiazole. In some cases, R⁴ is

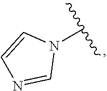 [SMILES: *n1ccnc1]

which is optionally substituted with one or more R⁹. In some cases, R⁴ is

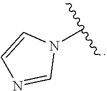 [SMILES: *n1ccnc1]

In some embodiments, for a compound or salt of Formula (I), R⁴ is selected from a 5-membered heteroaryl, wherein the 5-membered heteroaryl is selected from imidazole, thiazole, oxazole, oxadiazole, and thiadiazole, each of which is optionally substituted with one or more R⁹. In some cases, R⁴ is selected from a 5-membered heteroaryl, wherein the 5-membered heteroaryl is selected from imidazole, thiazole, and oxazole. In some cases, R⁴ is selected from a 5-membered heteroaryl, wherein the 5-membered heteroaryl is selected from imidazole and oxazole, each of which is optionally substituted with one or more R⁹. In some cases, R⁴ is selected from a 5-membered heteroaryl, wherein the 5-membered heteroaryl is selected from imidazole and thiazole, each of which is optionally substituted with one or more R⁹. In some cases, R⁴ is selected from imidazole, which is optionally substituted with one or more R⁹. In some cases, R⁴ is imidazole. In some cases, R⁴ is selected from a 6-membered heteroaryl, wherein the 6-membered heteroaryl is selected from pyrimidine, pyrazine, and pyridazine, each of which is optionally substituted with one or more R⁹. In some cases, R⁴ is selected from pyrimidine, which is optionally substituted with one or more R⁹. In some cases, R⁴ is pyrimidine.

In some embodiments, for a compound or salt of Formula (I), Formula (I-A), or Formula (I-B), R⁵ is hydrogen. In some cases, R⁵ is selected from C₁-C₆ alkyl. In some cases, R⁵ is selected from hydrogen and methyl. In some cases, R⁵ is methyl.

In some embodiments, for a compound or salt of Formula (I) or Formula (I-A), R⁷ is selected from an optionally substituted saturated 4- to 8-membered heterocycle. In some cases, the saturated 4- to 8-membered heterocycle is optionally substituted with one or more R⁸. In some cases, R⁷ is selected from an optionally substituted saturated 5-membered heterocycle. In some cases, the 5-membered heterocycle is optionally substituted with one or more R⁸. In some cases, R⁷ is selected from an optionally substituted saturated 6-membered heterocycle. In some cases, the 6-membered heterocycle is optionally substituted with one or more R⁸. In some cases, R⁷ is selected from an optionally substituted saturated 4- to 6-membered heterocycle. In some cases, R⁷ is selected from an optionally substituted saturated 5- to 6-membered heterocycle. In some cases, R⁷ is selected from an optionally substituted saturated 4-membered heterocycle. In some cases, R⁷ is selected from an optionally substituted saturated 5-membered heterocycle. In some cases, R⁷ is selected from an optionally substituted saturated 6-membered heterocycle. In some cases, R⁷ is selected from an optionally substituted saturated 4- to 6-membered heterocycle, wherein the saturated 4- to 6-membered heterocycle has at least one heteroatom selected from oxygen, nitrogen, and sulfur. In some cases, the saturated 4- to 6-membered heterocycle has at most one heteroatom. In some cases, R⁷ is selected from

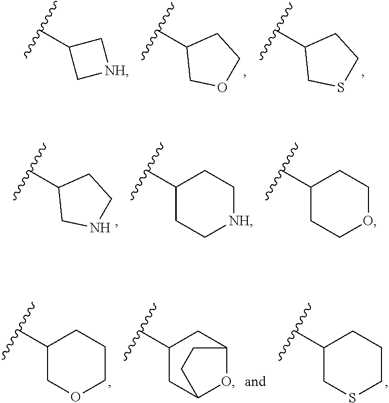

wherein each is optionally substituted with one or more substituents independently selected from R⁸. In some cases, R⁷ is selected from

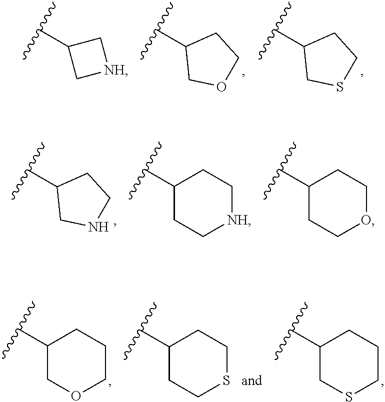

wherein each is optionally substituted with one or more substituents independently selected from R⁸. In some cases, R⁷ is selected from

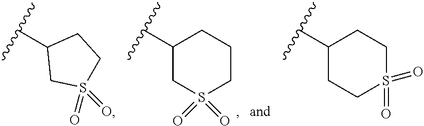 [SMILES: *C1CCS(=O)(=O)C1 ; *C1CCCS(=O)(=O)C1 ; *C1CCS(=O)(=O)CC1]

wherein each is optionally substituted with one or more substituents independently selected from R⁸. In some cases, R⁷ is selected from

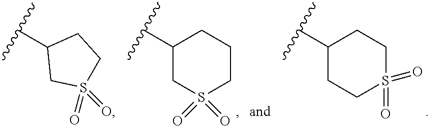 [SMILES: *C1CCS(=O)(=O)C1 ; *C1CCCS(=O)(=O)C1 ; *C1CCS(=O)(=O)CC1]

In some embodiments, for a compound or salt of Formula (I) or Formula (I-A), R⁷ is selected from an optionally substituted saturated 4- to 6-membered heterocycle, wherein the optionally substituted saturated 4- to 6-membered heterocycle contains at least one nitrogen atom. In some cases, the optionally substituted saturated 4- to 6-membered heterocycle contains only 1 nitrogen atom. In some cases, the optionally substituted saturated 4- to 6-membered heterocycle contains at most 1 nitrogen atom. In some cases, R⁷ is selected from

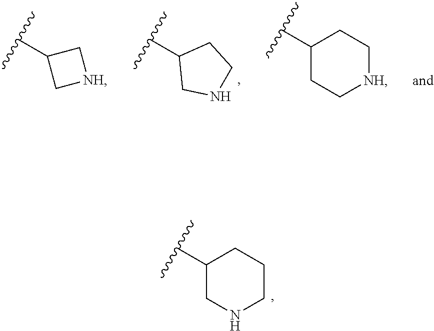 [SMILES: *C1CNC1 ; *C1CCNC1 ; *C1CCNCC1 ; *C1CCCNC1]

wherein each is optionally substituted with one or more substituents independently selected from R⁸. In some cases, R⁷ is selected from

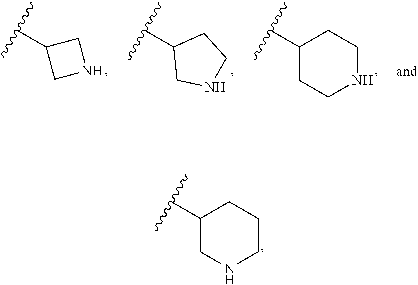 [SMILES: *C1CNC1 ; *C1CCNC1 ; *C1CCNCC1 ; *C1CCCNC1]

wherein each is optionally substituted with one or more substituents selected from halogen, —C(O)R²⁰ and —SO)₂(R²⁰). In some cases, R⁷ is selected from

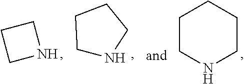

wherein each is substituted with —S(O)₂(R²⁰). In some cases, R⁷ is selected from

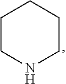

wherein the

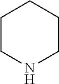

is substituted with —S(O)₂(R²⁰) and no other substituents, wherein R²⁰ is selected from C₁-C₆ alkyl. In some cases, R⁷ is selected from

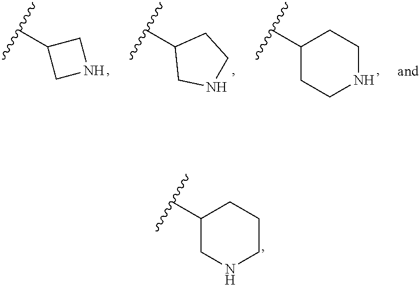 [SMILES: *C1CNC1 ; *C1CCNC1 ; *C1CCNCC1 ; *C1CCCNC1]

wherein each is substituted with —S(O)₂(R²⁰). In some cases, R⁷ is selected from

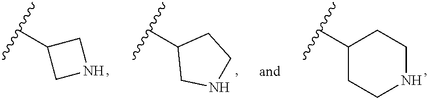 [SMILES: *C1CNC1 ; *C1CCNC1 ; *C1CCNCC1]

wherein each is substituted with —S(O)₂(R²⁰). In some cases, each R²⁰ is independently selected at each occurrence from hydrogen; C₁₋₆ alkyl and C₃₋₁₂ carbocycle, wherein the C₁₋₆ alkyl is optionally substituted with one or more —O—C₁₋₁₀ alkyl. In some cases, each R²⁰ is independently selected at each occurrence from hydrogen; C₁₋₆ alkyl and C₃₋₆ carbocycle. In some cases, each R²⁰ is independently selected at each occurrence from C₁₋₆ alkyl and C₃₋₆ carbocycle. In some cases, each R²⁰ is independently selected at each occurrence from C₁₋₆ alkyl. In some cases, each R²⁰ is independently selected at each occurrence from C₃₋₆ carbocycle. In some cases, R⁷ is selected from

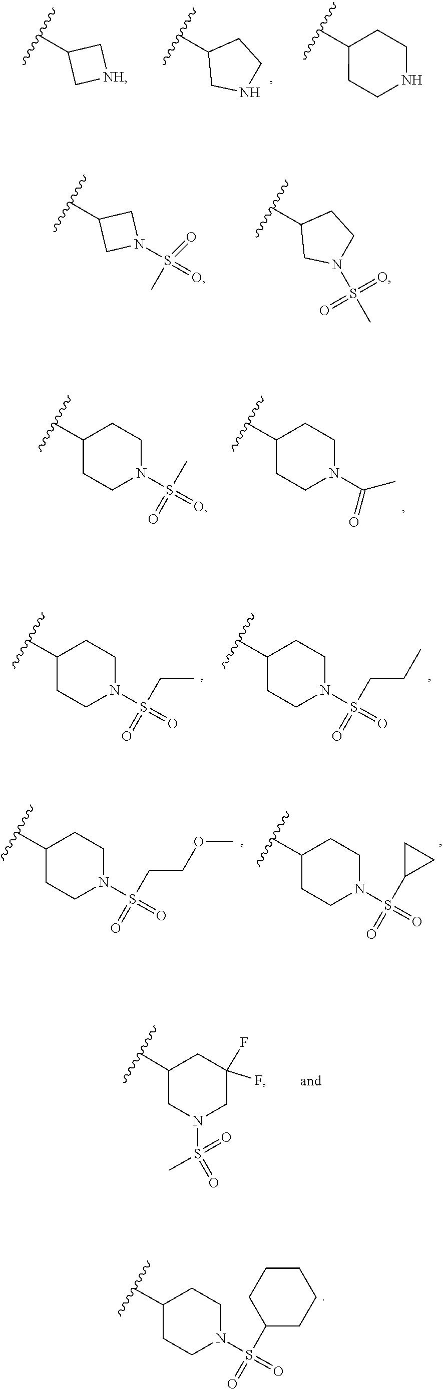

In some cases, R⁷ is selected from

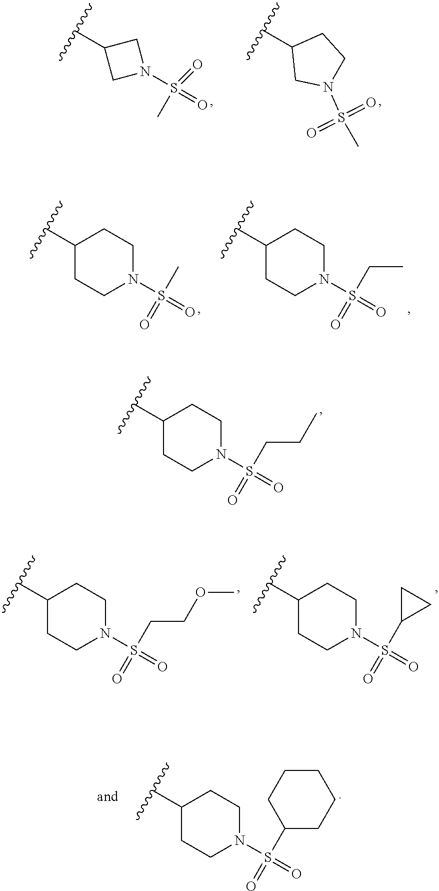

In some cases. R⁷ is selected from

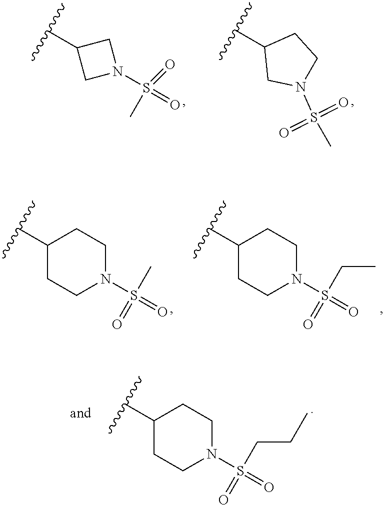

In some cases, R⁷ is selected from

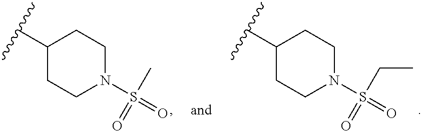 [SMILES: *C1CCN(S(C)(=O)=O)CC1 ; *C1CCN(S(=O)(=O)CC)CC1]

In some embodiments, for a compound or salt of Formula (I) or Formula (I-A), R⁷ is selected from an optionally substituted saturated 4- to 6-membered heterocycle, wherein the optionally substituted saturated 4- to 6-membered heterocycle contains at least one nitrogen atom. In some cases, R⁷ is selected from

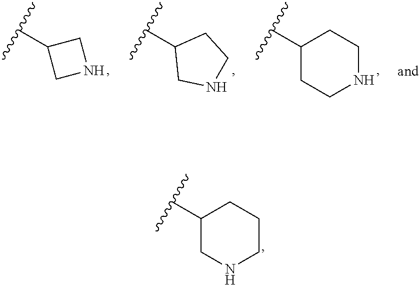 [SMILES: *C1CNC1 ; *C1CCNC1 ; *C1CCNCC1 ; *C1CCCNC1]

wherein each is optionally substituted with one or more substituents independently selected from R⁸. In some cases, R⁷ is a beta lactam 4- to 8-membered saturated heterocyle, wherein each is optionally substituted with one or more substituents independently selected from R⁸. In some cases, R⁷ is selected from

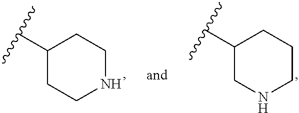 [SMILES: *C1CCNCC1 ; *C1CCCNC1]

wherein each is optionally substituted with one or more substituents independently selected from R⁸. In some cases, R⁷ is

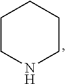

which is optionally substituted with one or more substituents independently selected from R⁸. In some cases, R⁷ is selected from

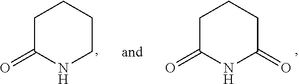 [SMILES: O=C1CCCCN1 ; O=C1CCCC(=O)N1]

each of which is optionally substituted with one or more substituents independently selected from R⁸. In some cases, R⁷ is

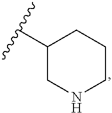 [SMILES: *C1CCCNC1]

which is optionally substituted with one or more substituents independently selected from R⁸. In some cases, each R⁸ is selected from C₁₋₆ alkyl, ═O, and saturated 4- to 8-membered heterocycle. In some cases, each R⁸ is selected from C₁₋₆ alkyl and ═O. In some cases, each R⁸ is selected from C₁₋₆ alkyl and ═O. In some cases, R⁷ is selected from

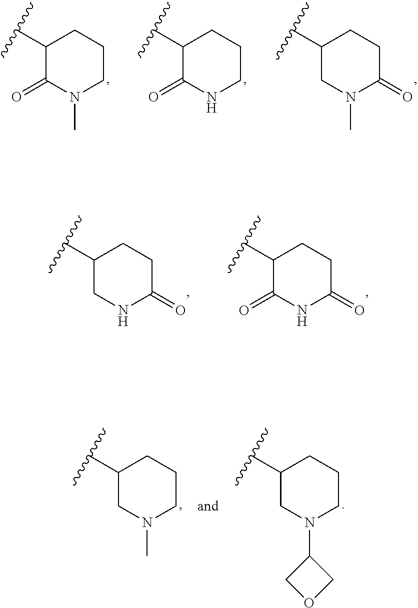

In some cases, R⁷ is selected from

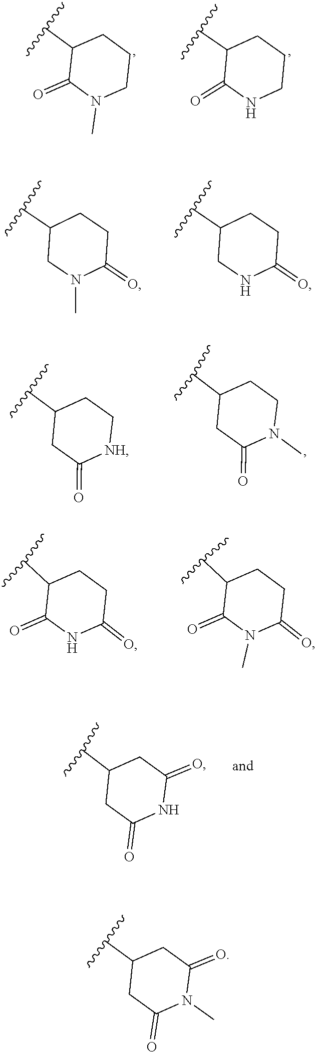

In some embodiments, for a compound or salt of Formula (I) or Formula (I-A), R⁷ is selected from an saturated 4- to 6-membered heterocycle, wherein the saturated 4- to 6-membered heterocycle contains at least one nitrogen atom and is substituted with at least one oxo, and is further optionally substituted with one or more R⁸. In some cases, R⁷ is selected from

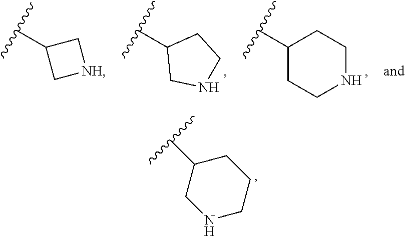 [SMILES: *C1CNC1 ; *C1CCNC1 ; *C1CCNCC1 ; *C1CCCNC1]

wherein each is substituted with at least one oxo and one or more substituents independently selected from R⁸. In some cases, R⁷ is selected from

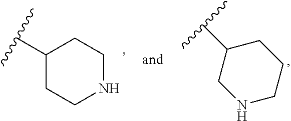 [SMILES: *C1CCNCC1 ; *C1CCCNC1]

wherein each is substituted with at least one oxo and further optionally substituted with one or more substituents independently selected from R⁸. In some cases, R⁷ is

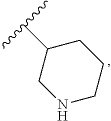 [SMILES: *C1CCCNC1]

which is substituted with at least one oxo and further optionally substituted with one or more substituents independently selected from R⁸. In some cases, each R⁸ is selected from C₁₋₆ alkyl, ═O, and saturated 4- to 8-membered heterocycle. In some cases, each R⁸ is selected from C₁₋₆ alkyl and ═O. In some cases, each R⁸ is selected from C₁₋₆ alkyl. In some cases, each R⁸ is selected from ═O. In some cases, R⁷ is selected from

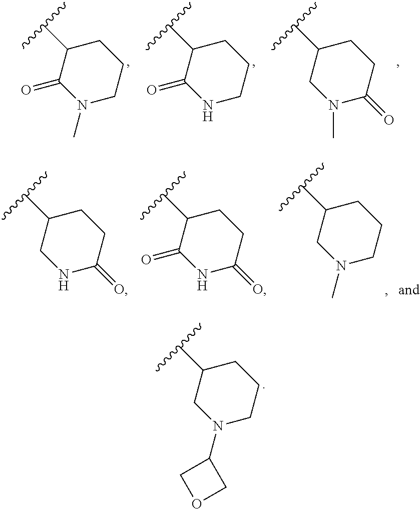

In some cases, R⁷ is selected from

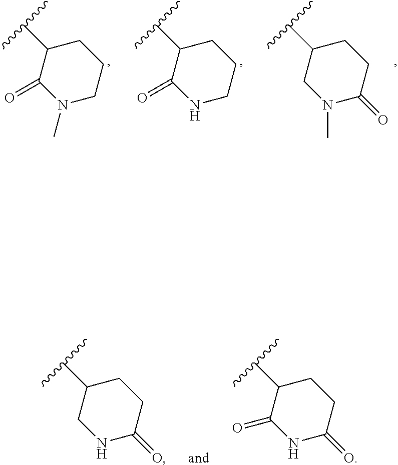

In some embodiments, for a compound or salt of Formula (I) or Formula (I-A), R⁷ is selected from an optionally substituted saturated 7- to 8-membered spiro heterocycle. In some cases, the spiro heterocycle has at least one nitrogen atom. In some cases, the spiro heterocycle has at least one oxygen atom. In some cases, R⁷ is selected from

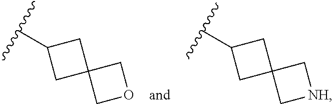 [SMILES: *C1CC2(COC2)C1 ; *C1CC2(CNC2)C1]

each of which is optionally substituted with one or more substituents independently selected from R⁸. In some cases, R⁷ is selected from

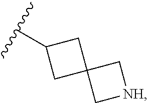 [SMILES: *C1CC2(CNC2)C1]

which is optionally substituted with one or more substituents independently selected from R⁸. In some cases, each R⁸ is independently selected from halogen, —OR²⁰, —SR²⁰, —N(R²¹)₂, —NO₂, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, each R⁸ is independently selected from C₁₋₆ alkyl, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, each R⁸ is independently selected from C₁₋₆ alkyl and —S(O)₂(R²⁰). In some cases, R⁷ is

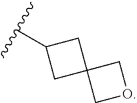 [SMILES: *C1CC2(COC2)C1]

In some cases, for a compounds or salt of Formula (I) or Formula (I-A), R⁷ is selected from an optionally substituted saturated 4- to 6-membered heterocycle, wherein the optionally substituted saturated 4- to 6-membered heterocycle contains at least one nitrogen atom and is substituted with at least one halogen, and least one —S(O)₂(R²⁰). In some cases, R⁷ is

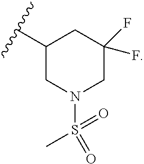 [SMILES: *C1CN(S(C)(=O)=O)CC(F)(F)C1]

In some embodiments, for a compound or salt of Formula (I), R⁷ is selected from an optionally substituted saturated 6-membered heterocycle, and C₆ cycloalkyl substituted with one or more R^{8A} and the C₆ cycloalkyl is further optionally substituted with one or more R^{8B}. In some cases, the saturated 6-membered heterocycle contains 1 nitrogen atom. In some cases, the saturated 6-membered heterocycle contains only 1 nitrogen atom and no further heteroatoms. In some cases, R⁷ is selected from

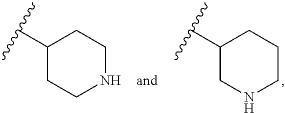 [SMILES: *C1CCNCC1 ; *C1CCCNC1]

each of which is optionally substituted with one or more substituents independently selected from R⁸; and C₆ cycloalkyl substituted with one or more R^{8A}. In some cases, R⁷ is selected from

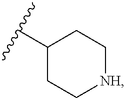 [SMILES: *C1CCNCC1]

which is optionally substituted with —C(O)R²⁰ and —S(O)₂(R²⁰); and C₆ cycloalkyl substituted with one or more halogen. In some cases, R⁷ is selected from

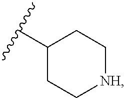 [SMILES: *C1CCNCC1]

which is optionally substituted with —S(O)₂(R²⁰); and C₆ cycloalkyl substituted with one or more halogen. In some cases, R⁷ is selected from

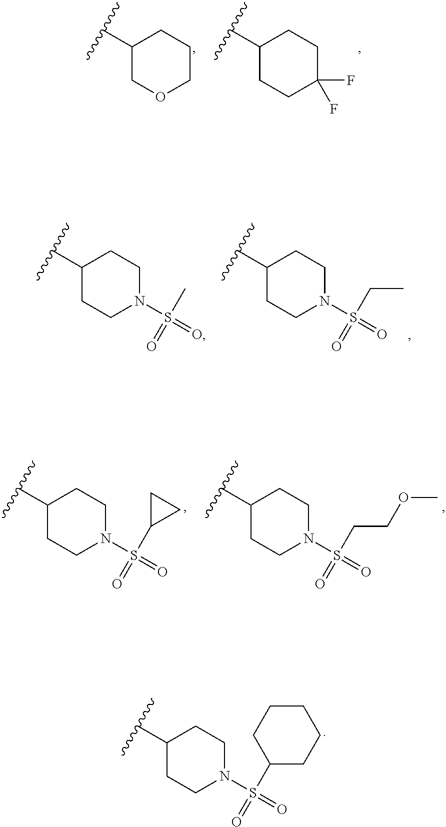

In some embodiments, for a compound or salt of Formula (I), R⁷ is selected from an optionally substituted saturated 6-membered heterocycle, and optionally substituted C₄ cycloalkyl, each of which is optionally substituted with one or more substituents independently selected from halogen, C₁₋₆haloalkyl, C₁₋₆ alkoxyalkyl, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁷ is selected from an optionally substituted saturated 6-membered heterocycle, and unsubstituted C₄ cycloalkyl, wherein the saturated 6-membered heterocycle is optionally substituted with one or more substituents independently selected from —S(O)₂(R²⁰). In some cases, R⁷ is selected from unsubstituted C₄ cycloalkyl,

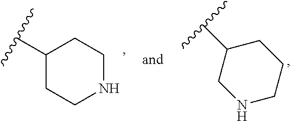 [SMILES: *C1CCNCC1 ; *C1CCCNC1]

each of which is optionally substituted with —S(O)₂(R²⁰) and halogen. In some cases, R⁷ is selected from unsubstituted C₄ cycloalkyl,

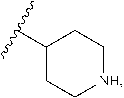 [SMILES: *C1CCNCC1]

each of which is optionally substituted with —S(O)₂(R²⁰). In some cases, R⁷ is selected from

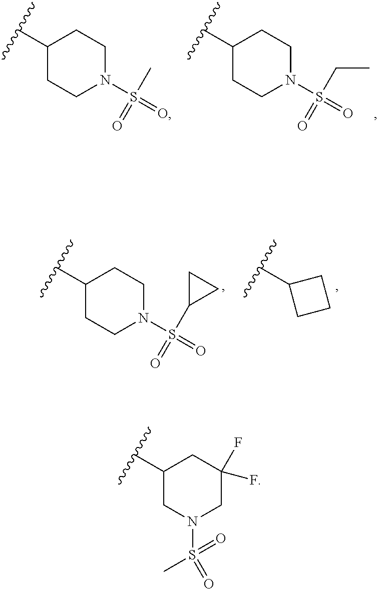 [SMILES: *C1CCN(S(C)(=O)=O)CC1 ; *C1CCN(S(=O)(=O)CC)CC1 ; *C1CCN(S(=O)(=O)C2CC2)CC1 ; *C1CCC1 ; *C1CN(S(C)(=O)=O)CC(F)(F)C1]

In some cases, R⁷ is selected from

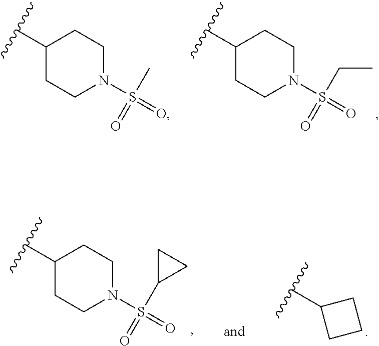 [SMILES: *C1CCN(S(C)(=O)=O)CC1 ; *C1CCN(S(=O)(=O)CC)CC1 ; *C1CCN(S(=O)(=O)C2CC2)CC1 ; *C1CCC1]

In some embodiments, for a compound or salt of Formula (I) or Formula (I-A), R⁷ is selected from an optionally substituted saturated 4- to 8-membered heterocycle, wherein the optionally substituted saturated 4- to 8-membered heterocycle contains at least one oxygen atom. In some cases, the optionally substituted saturated 4- to 8-membered heterocycle contains only 1 oxygen atom. In some cases, the optionally substituted saturated 4- to 6-membered heterocycle contains at most 1 oxygen atom. In some cases, R⁷ is selected from

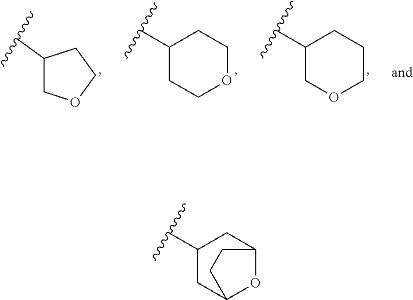 [SMILES: *C1CCOC1 ; *C1CCOCC1 ; *C1CCCOC1 ; *C1CC2CCC(C1)O2]

wherein each is optionally substituted with one or more substituents independently selected from R⁸. In some cases, R⁷ is selected from an optionally substituted saturated 4- to 6-membered heterocycle, wherein the optionally substituted saturated 4- to 6-membered heterocycle contains at least one oxygen atom. In some cases, R⁷ is selected from

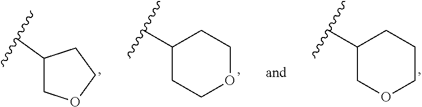 [SMILES: *C1CCOC1 ; *C1CCOCC1 ; *C1CCCOC1]

each is optionally substituted with one or more substituents independently selected from R⁸. In some cases, R⁷ is

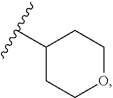 [SMILES: *C1CCOCC1]

which is optionally substituted with one or more substituents independently selected from R⁸. In some cases, R⁷ is

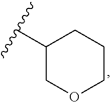 [SMILES: *C1CCCOC1]

which is optionally substituted with one or more substituents independently selected from R⁸.

In some embodiments, for a compound or salt of Formula (I) or Formula (I-A), R⁷ is an optionally substituted 6-membered saturated heterocycle containing one oxygen atom and no further heteroatoms. In some cases, the heterocycle is tetrahydropyran. In some cases, each R⁸ is independently selected from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, each R⁸ is independently selected from halogen, —OR²⁰, ═O, —CN, C₁₋₆ aminoalkyl, C₁₋₆ hydroxyalkyl, C₁₋₆ cyanoalkyl, C₁₋₆ haloalkyl, C₁₋₆ alkoxyalkyl, and C₁₋₆ alkyl. In some cases, each R⁸ is independently selected from halogen and C₁₋₆ alkyl. In some cases, the heterocycle is unsubstituted. In some cases, the heterocycle is substituted. In some cases, R⁷ is selected from

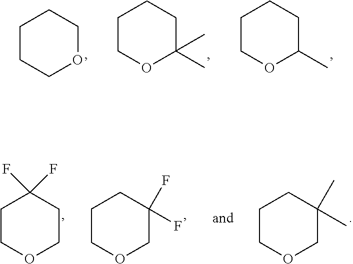

In some embodiments, for a compound or salt of Formula (I) or Formula (I-A), R⁷ is selected from an optionally substituted saturated 8-membered heterocycle, wherein the optionally substituted saturated 8-membered heterocycle contains at least one oxygen atom. In some cases, the saturated 8-membered heterocycle is a bridged heterocycle. In some cases, R⁷ is

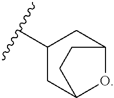 [SMILES: *C1CC2CCC(C1)O2]

In some embodiments, for a compound or salt of Formula (I) or Formula (I-A), R⁷ is selected from an optionally substituted saturated 4- to 6-membered heterocycle, wherein the optionally substituted saturated 4- to 6-membered heterocycle contains at least one sulfur atom. In some cases, the optionally substituted saturated 4- to 6-membered heterocycle contains only 1 sulfur atom. In some cases, the optionally substituted saturated 4- to 6-membered heterocycle contains at most 1 sulfur atom. In some cases, R⁷ is selected from

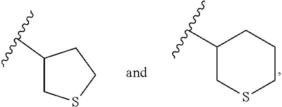 [SMILES: *C1CCSC1 ; *C1CCCSC1]

wherein each is optionally substituted with one or more substituents independently selected from R⁸. In some cases, R⁷ is selected from

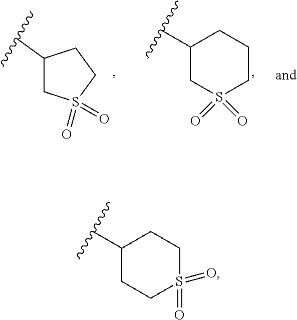 [SMILES: *C1CCS(=O)(=O)C1 ; *C1CCCS(=O)(=O)C1 ; *C1CCS(=O)(=O)CC1]

wherein each is optionally substituted with one or more substituents independently selected from R⁸. In some cases, R⁷ is selected from

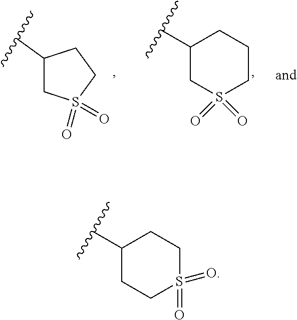 [SMILES: *C1CCS(=O)(=O)C1 ; *C1CCCS(=O)(=O)C1 ; *C1CCS(=O)(=O)CC1]

In some cases, R⁷ is selected from

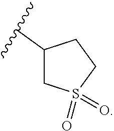 [SMILES: *C1CCS(=O)(=O)C1]

In some embodiments, for a compound or salt of Formula (I) or Formula (I-A), each R⁸ is independently selected from halogen, —OR²⁰, ═O, C₁₋₆ hydroxyalkyl, C₁₋₆ haloalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, —S(O)₂(NR²⁰₂), and —S(O)(NR²¹)N(R²¹)₂. In some cases, each R⁸ is independently selected from halogen, —OR²⁰, ═O, C₁₋₆ hydroxyalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, each R⁸ is independently selected from —C(O)R²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, each R⁸ is independently selected from —C(O)R²⁰, —C(O)N(R²¹)₂, —S(O)₂(R²⁰), and —S(O)(NR²¹)R²⁰. In some cases, each R⁸ is independently selected from halogen, —OR²⁰, C₁₋₆ hydroxyalkyl, C₁₋₆ haloalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, each R⁸ is independently selected from halogen, —OR²⁰, ═O, C₁₋₆ hydroxyalkyl, C₁ haloalkyl, C₃₋₆ haloalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, R⁸ is selected from halogen, ═O, C₁₋₆ hydroxyalkyl, C₁ haloalkyl, C₁₋₆ alkyl, —C(O)R²⁰, —S(O)₂(R²⁰), —S(O)(R²⁰), —S(O)(NR²¹)R²⁰, and —S(O)(NR²¹)N(R²¹)₂. In some cases, R⁸ is selected from ═O, —C(O)R²⁰, —S(O)₂(R²⁰), —S(O)(R²⁰), —S(O)(NR²¹)R²⁰, and —S(O)(NR²¹)N(R²¹)₂. In some cases, R⁸ is selected from —S(O)₂(R²⁰), —S(O)₂(NR²⁰₂), —S(O)(NR²¹)R²⁰, and —S(O)(NR²¹)N(R²¹)₂. In some cases, R⁸ is selected from —S(O)₂(R²⁰). In some cases, R⁸ is selected from —C(O)N(R²¹)₂, —S(O)₂(R²⁰), —S(O)(NR²¹)R²⁰, and —C(O)R²⁰. In some cases, R⁸ is selected from —C(O)N(R²¹)₂, —S(O)₂(R²⁰), and —S(O)(NR²¹)R²⁰. In some cases, R⁸ is —S(O)₂(NR²⁰₂). In some cases, R⁸ is selected from —S(O)₂(R²⁰) and —C(O)R²⁰. In some cases, R⁸ is —C(O)N(R²¹)₂. In some cases, R⁸ is —S(O)₂(R²⁰). In some cases, R⁸ is —S(O)(NR²¹)R²⁰. In some cases, R⁸ is selected from halogen, —OR²⁰, and ═O. In some cases, R⁸ is selected from C₁₋₆ hydroxyalkyl, C₁₋₆ haloalkyl, and C₁₋₆ alkyl. In some cases, R⁸ is selected from halogen. In some cases, each R²⁰ is independently selected at each occurrence from hydrogen; C₁₋₆ alkyl, C₃₋₁₂ carbocycle, and 3- to 12-membered heterocycle. In some cases, each R²⁰ is independently selected at each occurrence from hydrogen; C₁₋₆ alkyl and C₃₋₁₂ carbocycle, wherein the C₁₋₆ alkyl is optionally substituted with one or more —O—C₁₋₁₀ alkyl. In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, each R²⁰ is independently selected at each occurrence from hydrogen and C₁₋₆ alkyl. In some cases, each R²¹ is independently selected at each occurrence from hydrogen, C₁₋₆ alkyl, C₃₋₁₂ carbocycle, and 3- to 12-membered heterocycle. In some cases, each R²¹ is independently selected at each occurrence from hydrogen and C₁₋₆ alkyl. In some cases, R⁷ is selected from

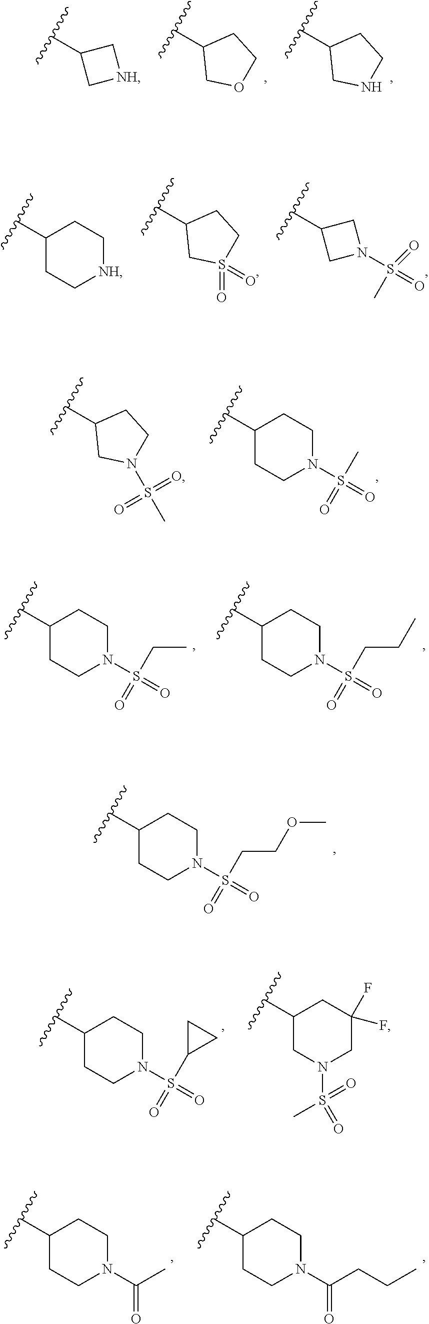

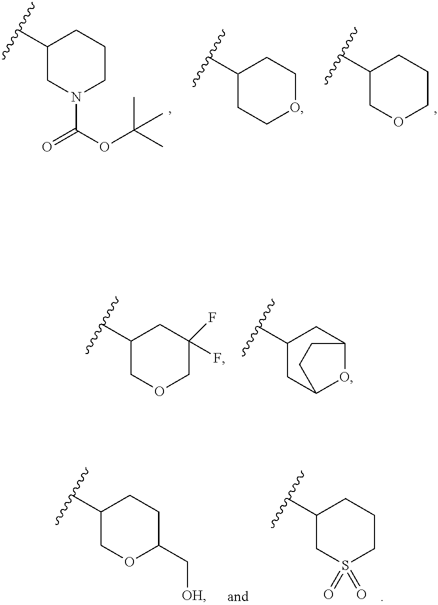

In some cases, R⁷ is selected from

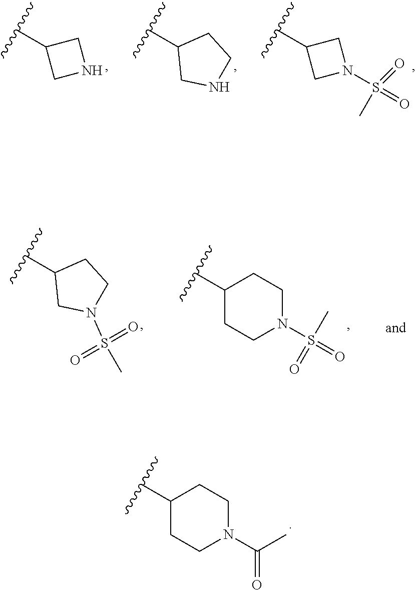

In some cases, R⁷ is selected from

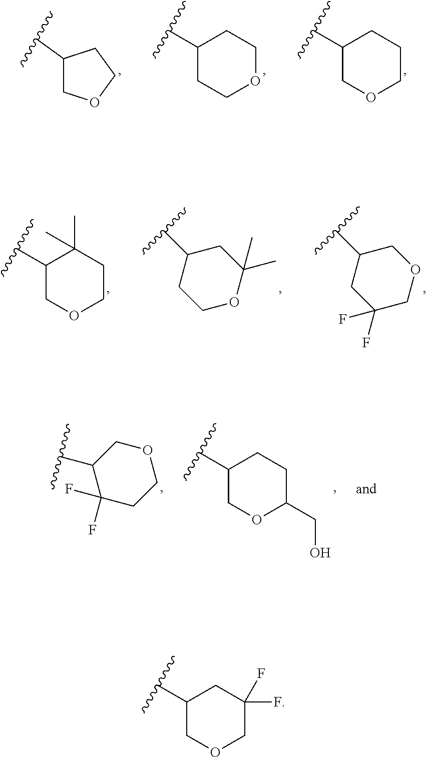

In some cases, R⁷ is selected from,

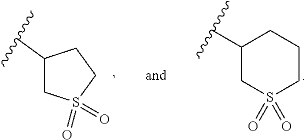 [SMILES: *C1CCS(=O)(=O)C1 ; *C1CCCS(=O)(=O)C1]

In some cases, R⁷ is selected from

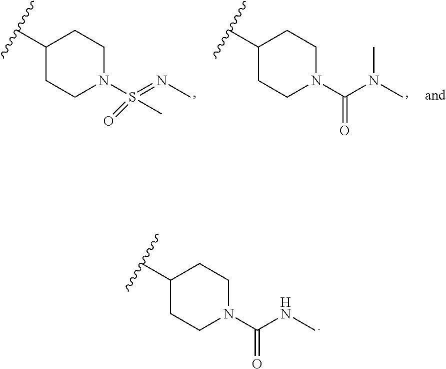 [SMILES: *C1CCN(S(C)(=O)=NC)CC1 ; *C1CCN(C(=O)N(C)C)CC1 ; *C1CCN(C(=O)NC)CC1]

In some cases, R⁷ is selected from

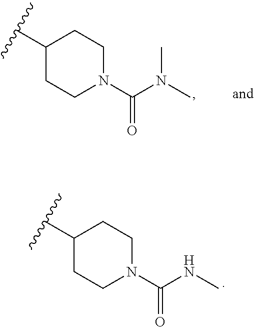 [SMILES: *C1CCN(C(=O)N(C)C)CC1 ; *C1CCN(C(=O)NC)CC1]

In some cases, R⁷ is selected from

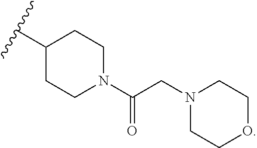 [SMILES: *C1CCN(C(=O)CN2CCOCC2)CC1]

In some cases, R⁷ is selected from

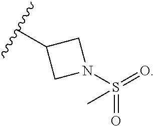 [SMILES: *C1CN(S(C)(=O)=O)C1]

In some cases, R⁷ is selected from

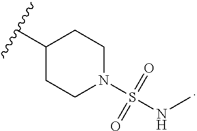 [SMILES: *C1CCN(S(=O)(=O)NC)CC1]

In some cases, R⁷ is selected from

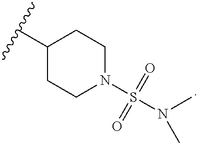 [SMILES: *C1CCN(S(=O)(=O)N(C)C)CC1]

In some cases, R⁷ is selected from

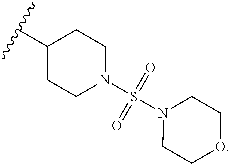 [SMILES: *C1CCN(S(=O)(=O)N2CCOCC2)CC1]

In some embodiments, for a compound or salt of Formula (I) or Formula (I-A), R⁸ is selected from —S(O)₂(NR²⁰₂) and —S(O)₂(R²⁰). In some cases, R⁸ is selected from —S(O)₂(NR²⁰₂). In some cases, R²⁰ is independently selected at each occurrence from hydrogen; C₁₋₆ alkyl, and 3-to 6-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from hydrogen and C₁₋₆ alkyl. In some cases, R²⁰ is independently selected at each occurrence from C₁₋₆ alkyl. In some cases, R⁸ is selected from

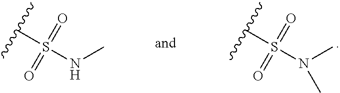

In some cases, R⁷ is

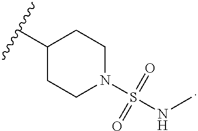 [SMILES: *C1CCN(S(=O)(=O)NC)CC1]

In some cases, R⁷ is

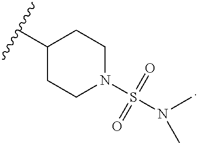 [SMILES: *C1CCN(S(=O)(=O)N(C)C)CC1]

In some embodiments, for a compound or salt of Formula (I) or Formula (I-A), R⁸ is selected from —S(O)₂(R²⁰). In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is 3- to 6-membered heterocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is 3- to 6-membered heterocycle. In some cases, R²⁰ is 6-membered heterocycle. In some cases, R²⁰ is saturated 6-membered heterocycle. In some cases, the heterocycle contains at least one oxygen atom. In some cases, the heterocycle contains at least one nitrogen atom. In some cases, the heterocycle contains at least one oxygen atom and at least one nitrogen atom. In some cases, R⁸ is

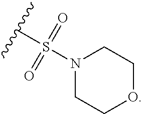 [SMILES: *S(=O)(=O)N1CCOCC1]

In some cases, R⁷ is selected from

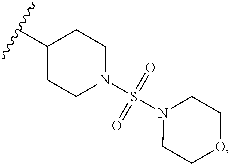 [SMILES: *C1CCN(S(=O)(=O)N2CCOCC2)CC1]

In some embodiments, for a compound or salt of Formula (I) or Formula (I-A), R⁸ is selected from —C(O)R²⁰. In some cases, R²⁰ is independently selected at each occurrence from C₁-6 alkyl which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl which is optionally substituted with one or more substituents independently selected from —O—C₁₋₁₀ alkyl, and 3- to 12-membered heterocycle. In some cases, R²⁰ is independently selected at each occurrence from C₁₋₆ alkyl which is optionally substituted with one or more substituents independently selected from halogen, —OH, —CN, —NO₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl which is optionally substituted with 3- to 6-membered heterocycle. In some cases, R²⁰ is independently selected at each occurrence from C₁₋₆ alkyl which is optionally substituted with 6-membered heterocycle. In some cases, the heterocycle contains at least one oxygen atom. In some cases, the heterocycle contains at least one nitrogen atom. In some cases, the heterocycle contains at least one oxygen atom and at least one nitrogen atom. In some cases, the 6-membered heterocycle is selected from morpholine. In some cases, R⁸ is

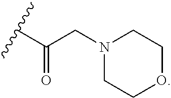 [SMILES: *C(=O)CN1CCOCC1]

In some cases, R⁸ is selected from

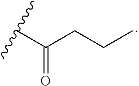 [SMILES: *C(=O)CCC]

In some cases, R⁸ is selected from

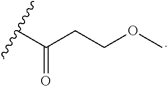 [SMILES: *C(=O)CCOC]

In some cases, R⁷ is selected from

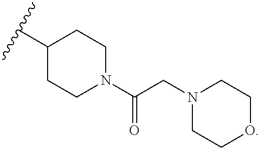 [SMILES: *C1CCN(C(=O)CN2CCOCC2)CC1]

In some cases, R⁷ is selected from

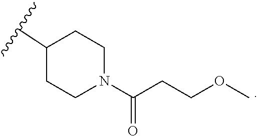 [SMILES: *C1CCN(C(=O)CCOC)CC1]

In some embodiments, for a compound or salt of Formula (I) or Formula (I-A), R⁸ is selected from —S(O)₂(R²⁰). In some cases, R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is independently selected at each occurrence from C₁₋₆ alkyl and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is independently selected at each occurrence from C₁₋₆ alkyl, which is optionally substituted with one or more substituents independently selected from halogen, —OH, —CN, —NO₂, —NH₂, —C₁₋₁₀ haloalkyl, and —O—C₁₋₁₀ alkyl. In some cases, R²⁰ is independently selected at each occurrence from C₁₋₆ alkyl, which is optionally substituted with one or more —O—C₁₋₁₀ alkyl. In some cases, R²⁰ is independently selected at each occurrence from C₁₋₆ alkyl. In some cases, R²⁰ is independently selected at each occurrence from methyl, ethyl and propyl. In some cases, R²⁰ is independently selected at each occurrence from C₃₋₆ carbocycle, which is optionally substituted with one or more substituents independently selected from halogen, —OH, —CN, —NO₂, —NH₂, C₁₋₁₀ alkyl, —C₁₋₁₀ haloalkyl, and —O—C₁₋₁₀ alkyl. In some cases, R²⁰ is independently selected at each occurrence from C₃₋₆ carbocycle, which is optionally substituted with one or more substituents independently selected from halogen, C₁₋₁₀ alkyl, —C₁₋₁₀ haloalkyl, and —O—C₁₋₁₀ alkyl. In some cases, R²⁰ is independently selected at each occurrence from C₃₋₆ carbocycle. In some cases, R⁸ is selected from

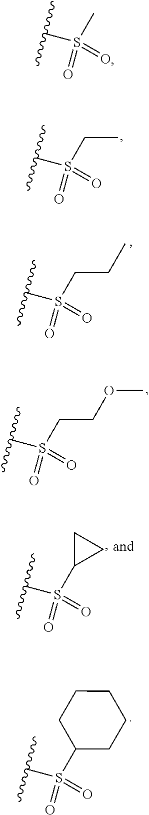

In some cases, R⁸ is selected from,

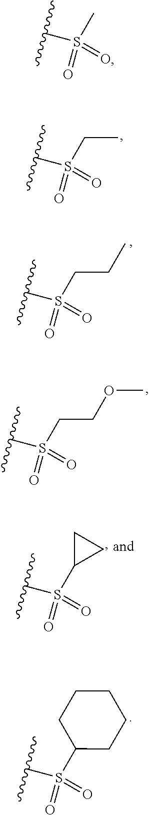

In some embodiments, for a compound or salt of Formula (I), R⁷ is selected from substituted C₆ cycloalkyl and optionally substituted saturated 6-membered heterocycle. In some cases, the heterocycle has at least one nitrogen atom. In some cases, the heterocycle has at least one oxygen atom. In some cases, the heterocycle is unsubstituted. In some cases, the heterocycle is substituted. In some cases, the heterocycle is selected from tetrahydropyran and piperidine. In some cases, each R^{8A} is independently selected from halogen and each R⁸ is independently selected from —C(O)N(R²¹)₂, —S(O)₂(R²⁰), and —S(O)₂(NR²⁰₂). In some cases, R⁷ is selected from

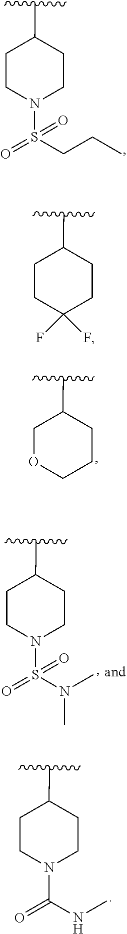

In some embodiments, for a compound or salt of Formula (I) or Formula (I-A), each R⁸ is independently selected from —C(O)R²⁰, —C(O)N(R²¹)₂, —S(O)₂(R²⁰), and —S(O)(NR²¹)R²⁰. In some cases, each R⁸ is independently selected from —C(O)N(R²¹)₂ and —S(O)₂(R²⁰), and —S(O)(NR²¹)R²⁰. In some cases, each R⁸ is independently selected from —S(O)₂(R²⁰), and —S(O)(NR²¹)R²⁰. In some cases, each R⁸ is —C(O)N(R²¹)₂. In some cases, each R⁸ is —S(O)(NR²¹)R²⁰. In some cases, R²¹ is independently selected at each occurrence from hydrogen; C₁₋₆ alkyl, and C₃₋₁₂ carbocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²¹ is independently selected at each occurrence from C₁₋₆ alkyl, which is optionally substituted with one or more substituents independently selected from halogen, —OH, —CN, —NO₂, —NH₂, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²¹ is independently selected at each occurrence from C₁₋₆ alkyl, which is optionally substituted with one or more substituents independently selected from halogen, —OH, —CN, —NO₂, —NH₂, —O—C₁₋₁₀ alkyl. In some cases, R²¹ is independently selected at each occurrence from C₁₋₆ alkyl. In some cases, R²¹ is methyl. In some cases, R⁸ is selected from,

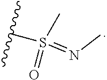 [SMILES: *S(C)(=O)=NC]

In some cases, R⁸ is selected from,

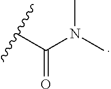 [SMILES: *C(=O)N(C)C]

In some cases, R⁸ is selected from,

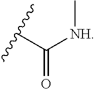 [SMILES: *C(=O)NC]

In some embodiments, for a compound or salt of Formula (I), when R⁴ is imidazole, R³ is hydrogen, R⁷ is pyrrolidine, the pyrrolidine is optionally substituted with one or more substituents independently selected from halogen, —OR², ═O, C₁₋₆ hydroxyalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, when R⁴ is imidazole, R³ is hydrogen, R⁷ is pyrrolidine, the pyrroldine is optionally substituted with one or more substituents independently selected from halogen, —OR²⁰, C₁₋₆ hydroxyalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, when R⁴ is imidazole, R³ is hydrogen, R⁷ is not substituted pyrrolidine. In some cases, when R⁴ is imidazole, R³ is hydrogen, R⁷ is not pyrrolidine. In some cases, R⁷ is not substituted pyrrolidine. In some cases, R⁷ is not pyrrolidine.

In some embodiments, for a compound or salt of Formula (I), R⁷ is selected from C₆ cycloalkyl substituted with one or more R^{8A} and the C₆ cycloalkyl is further optionally substituted with one or more R^{8B}.

In some embodiments, for a compound or salt of Formula (I), R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), and —S(O)₂(NR²⁰₂). In some cases, R^{8A} is selected at each occurrence from halogen, C₁₋₆ haloalkyl, —N(R²¹)C(O)R²⁰, —N(R²¹)C(O)N(R²¹)₂, —S(O)₂(R²⁰), —S(O)(R²⁰), and —S(O)₂(NR²⁰₂). In some cases, R^{8A} is selected at each occurrence from halogen, C₁₋₆haloalkyl, —N(R²¹)C(O)R²⁰, and —N(R²¹)C(O)N(R²¹)₂. In some cases, R^{8A} is selected at each occurrence from halogen and C₁₋₆ haloalkyl. In some cases, R^{8A} is selected at each occurrence from —N(R²¹)C(O)R²⁰ and —N(R²¹)C(O)N(R²¹)₂. In some cases, R^{8A} is selected at each occurrence from halogen.

In some embodiments, for a compound or salt of Formula (I), R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, R^{8A} is independently selected at each occurrence from halogen, C₁₋₆ haloalkyl, —C(O)R²⁰, —S(O)₂(R²⁰), —S(O)(R²⁰), —S(O)(NR²¹)R²⁰, and —S(O)(NR²¹)N(R²¹)₂. In some cases, R^{8A} is independently selected at each occurrence from halogen, —C(O)R²⁰, and —S(O)₂(R²⁰). In some cases, R²⁰ is independently selected at each occurrence from hydrogen, and C₁₋₆ alkyl. In some cases, R²¹ is independently selected at each occurrence from hydrogen, and C₁₋₆ alkyl.

In some embodiments, for a compound or salt of Formula (I), R^{8B} is independently selected each occurrence from —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, and C₁₋₆ alkyl. In some cases, R^{8B} is selected each occurrence from —OR²⁰, —SR²⁰, —N(R²¹)₂, —NO₂, ═O, ═S, ═N(R²⁰), and —CN. In some cases, R^{8B} is selected each occurrence from —OR²⁰, —SR²⁰, and —N(R²¹)₂. In some cases, R^{8B} is selected each occurrence from —OR²⁰. In some cases, R²⁰ is independently selected at each occurrence from hydrogen, and C₁₋₆ alkyl. In some cases, R²¹ is independently selected at each occurrence from hydrogen, and C₁₋₆ alkyl.

In some embodiments, for a compound or salt of Formula (I), R⁷ is selected from C₆ cycloalkyl substituted with one or more R^{8A} and the C₆ cycloalkyl is further optionally substituted with one or more R^{8B}. In some cases, R⁷ is

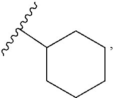 [SMILES: *C1CCCCC1]

which is substituted with one or more R^{8A}. In some cases, R⁷ is

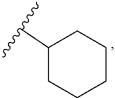 [SMILES: *C1CCCCC1]

which is substituted with two R^{8A}. In some cases, R^{8A} is independently selected at each occurrence from halogen, and C₁₋₆ haloalkyl. In some cases, R⁷ is selected from

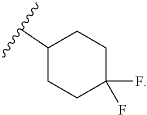 [SMILES: *C1CCC(F)(F)CC1]

In some embodiments, for a compound or salt of Formula (I), R⁷ is selected from C₈₋₁₀ cycloalkyl, each of which is optionally substituted with one or more R⁸. In some cases, R⁷ is selected from

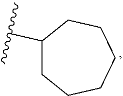 [SMILES: *C1CCCCCC1]

which is optionally substituted with one or more R⁸. In some cases, R⁷ is selected from C₈ cycloalkyl, which is optionally substituted with one or more R⁸. In some cases, the C₈ cycloalkyl is selected from

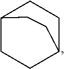

which is optionally substituted with one or more R⁸. In some cases, R⁷ is

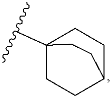 [SMILES: *C12CCC(CC1)CC2]

which is optionally substituted with one or more R⁸. In some cases, R⁷ is

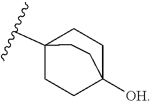 [SMILES: *C12CCC(O)(CC1)CC2]

In some cases, R⁷ is selected from C₁₀ cycloalkyl, which is optionally substituted with one or more R⁸. In some cases, R⁷ is

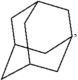

which is optionally substituted with one or more R⁸. In some cases, R⁷ is selected from

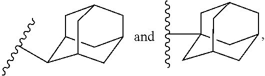 [SMILES: *C1C2CC3CC(C2)CC1C3 ; *C12CC3CC(CC(C3)C1)C2]

which is optionally substituted with one or more R⁸. In some cases, each R⁸ is independently selected at each occurrence from halogen, —OR²⁰, —N(R²¹)₂, ═O, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle. In some cases, each R⁸ is independently selected at each occurrence from —OR²⁰, C₁-6 aminoalkyl, C₁₋₆ hydroxyalkyl, C₁₋₆ cyanoalkyl, C₁₋₆ haloalkyl, C₁₋₆ alkoxyalkyl, and C₁₋₆ alkyl. In some cases, each R⁸ is independently selected at each occurrence from —OR²⁰, and C₁₋₆ alkyl. In some cases, each R⁸ is independently selected at each occurrence from —OH, and —O—C₁₋₆ alkyl. In some cases, R⁷ is

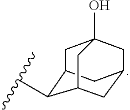 [SMILES: *C1C2CC3CC1CC(O)(C3)C2]

In some cases, R⁷ is

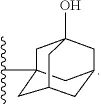 [SMILES: *C12CC3CC(C1)CC(O)(C3)C2]

In some embodiments, for a compound or salt of Formula (I), R⁷ is selected from C₃₋₅ cycloalkyl, C₇₋₈ cycloalkyl, and C₉₋₁₀ cycloalkyl, each of which is optionally substituted with one or more R⁸. In some cases, R⁷ is selected from C₃ cycloalkyl, C₅ cycloalkyl, C₇₋₈ cycloalkyl, and C₉₋₁₀ cycloalkyl, each of which is optionally substituted with one or more R⁸. In some cases, R⁷ is selected from

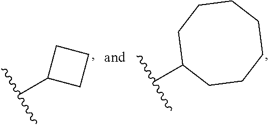 [SMILES: *C1CCC1 ; *C1CCCCCCC1]

each of which is optionally substituted with one or more R⁸. In some cases, R¹ is CR¹¹ and R⁷ is selected from C₃₋₅ cycloalkyl, and C₇₋₈ cycloalkyl, each of which is optionally substituted with one or more R⁸. In some cases, R¹¹ is hydrogen. In some cases, R⁸ is independently selected at each occurrence from each halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁸ is independently selected at each occurrence from each halogen, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁸ is selected from —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁸ is —S(O)₂(R²⁰). In some cases, when R¹ is N and R³ is imidazole, R⁷ is not

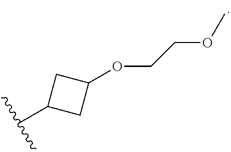 [SMILES: *C1CC(OCCOC)C1]

In some cases, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, and —C₁₋₁₀ haloalkyl. In some cases, when R¹ is N, R³ is imidazole, R⁷ is C₄ cycloalkyl, and R⁸ is —OR²⁰, R²⁰ is selected from C₁ alkyl, C₃₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle. In some cases, when R¹ is N, R³ is imidazole, R⁷ is C₄ cycloalkyl, and R⁸ is —OR²⁰, R²⁰ is selected from C₁₋₆ alkyl, C₂₋₆ alkenyl, C₂₋₆ alkynyl, each of which is optionally substituted with one or more substituents independently selected from halogen, —OH, —CN, —NO₂, —NH₂, C₁₋₁₀ alkyl, —C₁₋₁₀ haloalkyl, —O—C₂₋₁₀ alkyl, C₂₋₁₀ alkenyl, C₂₋₁₀ alkynyl, C₃₋₁₂ carbocycle, and 3- to 12-membered heterocycle. In some cases, R⁷ is

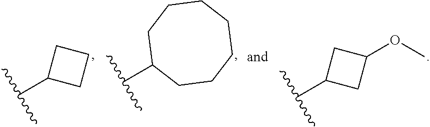

In some embodiments, for a compound or salt of Formula (I), R⁷ is selected from an optionally substituted C₅ cycloalkyl. In some cases, the C₅ cycloalkyl is substituted. In some cases, R⁷ is C₅ cycloalkyl substituted with one or more fluorine atoms. In some cases, R⁷ is selected

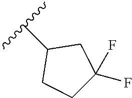 [SMILES: *C1CCC(F)(F)C1]

In some embodiments, for a compound or salt of Formula (I), when R¹ is N, R³ is imidazole, R⁷ is C₄ cycloalkyl, and R⁸ is —OR²⁰, the C₁₋₆ alkyl of R²⁰ is selected from C₁ alkyl and C₃₋₆ alkyl.

In some embodiments, for a compound or salt of Formula (I) or Formula (I-A), each R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂.

In some embodiments, for a compound or salt of Formula (I) or Formula (I-A), each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, and 3- to 12-membered heterocycle. In some cases, each R⁹ is independently selected from halogen, —OH, —CN, —NO₂, —NH₂, —NHC₁₋₁₀ alkyl, and —N(C₁₋₁₀ alkyl)₂. In some cases, each R⁹ is independently selected from halogen, —OH, —CN, and —NH₂. In some cases, each R⁹ is independently selected from C₁₋₁₀ alkyl, —C₁₋₁₀ haloalkyl, —O—C₁₋₁₀ alkyl, C₂₋₁₀ alkenyl, and C₂₋₁₀ alkynyl. In some cases, each R⁹ is independently selected from C₁₋₁₀ alkyl, —C₁₋₁₀ haloalkyl, and —O—C₁₋₁₀ alkyl. In some cases, each R⁹ is independently selected from C₃₋₁₂ carbocycle. In some cases, each R⁹ is independently selected from 3- to 12-membered heterocycle.

In some embodiments, for a compound or salt of Formula (I) or Formula (I-A), R¹¹ is selected from hydrogen, halogen, —OH, —CN, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹¹ is selected from hydrogen, halogen, —OH, —CN, —O—C₁₋₁₀ alkyl, C₁-C₆ alkyl, and C₁-C₆ haloalkyl. In some cases, R¹¹ is selected from hydrogen and C₁-C₆ alkyl. In some cases, R¹¹ is selected from hydrogen, halogen, —OH, —CN, and —O—C₁₋₁₀ alkyl. In some cases, R¹¹ is selected from hydrogen, halogen, —OH, and —CN. In some cases, R¹¹ is hydrogen. In some cases, R¹ is CH.

In some embodiments, for a compound or salt of Formula (I) or Formula (I-A), R¹² is selected from hydrogen, halogen, —OH, —CN, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl. In some cases, R¹² is selected from hydrogen, halogen, —OH, —CN, —O—C₁₋₁₀ alkyl, C₁-C₆ alkyl, and C₁-C₆ haloalkyl. In some cases, R¹² is selected from hydrogen and C₁-C₆ alkyl. In some cases, R¹² is selected from hydrogen, halogen, —OH, —CN, and —O—C₁₋₁₀ alkyl. In some cases, R¹² is selected from hydrogen, halogen, —OH, and —CN. In some cases, R¹² is hydrogen. In some cases, R² is CH.

In some embodiments, for a compound or salt of Formula (I) or Formula (I-A), R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle. In some cases, R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₁₋₆ alkyl, and C₃₋₆ carbocycle, wherein the C₁₋₆ alkyl, and C₃₋₆ carbocycle are each optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from hydrogen; C₁₋₆ alkyl, and C₃₋₁₂ carbocycle. In some cases, R¹⁶ is selected from C₁₋₆ alkyl and C₃₋₆ carbocycle. In some cases, R¹⁶ is selected from methyl and phenyl. In some cases, R¹⁶ is selected from C₁₋₆ alkyl, wherein the C₁₋₆ alkyl is optionally substituted with one or more substituents independently selected from halogen, —OH, —CN, —NO₂, —NH₂, —NHC₁₋₁₀ alkyl, —N(C₁₋₁₀ alkyl)₂, and —O—C₁₋₁₀ alkyl. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle. In some cases, R¹⁶ is selected from C₃₋₆ carbocycle, wherein the C₃₋₆ carbocycle is optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl.

In some embodiments, for a compound or salt of Formula (I), Formula (I-A), or Formula (I-B), R²⁰ is independently selected at each occurrence from hydrogen, C₁₋₆ alkyl, and C₃₋₁₂ carbocycle. In some cases, R²⁰ is independently selected at each occurrence from hydrogen and C₁₋₆ alkyl. In some cases, R²⁰ is methyl. In some cases, R²⁰ is ethyl. In some cases, R²⁰ is propyl. In some cases, R²⁰ is butyl. In some cases, R²⁰ is hexyl.

In some embodiments, for a compound or salt of Formula (I), Formula (I-A), or Formula (I-B), R²¹ is independently selected at each occurrence from hydrogen, C₁₋₆ alkyl, and C₃₋₁₂ carbocycle. In some cases, R²¹ is independently selected at each occurrence from hydrogen and C₁₋₆ alkyl. In some cases, R²¹ is methyl. In some cases, R²¹ is ethyl. In some cases, R²¹ is propyl. In some cases, R²¹ is butyl. In some cases, R²¹ is hexyl.

In some embodiments, for a compound or salt of Formula (I-B), R³ is selected from hydrogen, halogen, —CN, —OR¹⁶, C₁-C₆ alkyl, and 3- to 12-membered heterocycle, wherein the C₁-C₆ alkyl, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle. In some cases, R³ is selected from hydrogen, —OR¹⁶, —SO₂R¹⁶, optionally substituted C₁-C₆ alkyl, and 3- to 12-membered heterocycle, wherein the C₁-C₆ alkyl and 3- to 12-membered heterocycle are each optionally substituted with one or more substituents independently selected from halogen, —OH, —CN, —NO₂, —NH₂, —NHC₁₋₁₀ alkyl, C₁-C₆ alkyl, —N(C₁₋₁₀ alkyl)₂, —C₁₋₁₀ haloalkyl, and —O—C₁₋₁₀ alkyl. In some cases, R³ is selected from hydrogen, —OR¹⁶, C₁-C₆ alkyl, and 5- to 6-membered heterocycle, wherein the 5- to 6-membered heterocycle is optionally substituted with one or more substituents independently selected from C₁-C₆ alkyl. In some cases, R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl. In some cases, R¹⁶ is selected from hydrogen; C₁₋₆ alkyl, and C₃₋₁₂ carbocycle.

In some embodiments, for a compound or salt of Formula (I-B), R⁵ is hydrogen.

In some embodiments, for a compound or salt of Formula (I-B), R¹ is selected from N and CH. In some case, R¹ is CH. In some cases, R² is CH. In some cases, R¹ is N. In some cases, R² is N.

In some embodiments, for a compound or salt of Formula (I) or Formula (I-B), R⁷ is selected from a saturated 4- to 8-membered heterocycle, wherein the saturated 4- to 8-membered heterocycle is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the saturated 4- to 8-membered heterocycle is further optionally substituted with one or more R⁸. In some cases, the saturated heterocycle of R⁷ is selected from

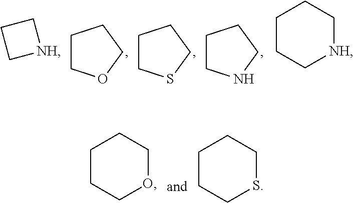

In some cases, the saturated heterocycle of R⁷ is selected from

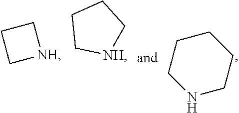

In some cases, the saturated heterocycle of R⁷ is selected from

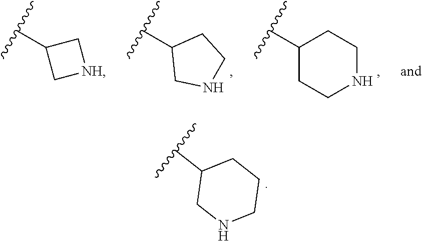 [SMILES: *C1CNC1 ; *C1CCNC1 ; *C1CCNCC1 ; *C1CCCNC1]

In some cases, the at least one substitutent of R⁷ is selected from —S(O)₂(R²⁰), and —S(O)₂(NR²⁰₂). In some cases, the at least one substituent of R⁷ is selected from

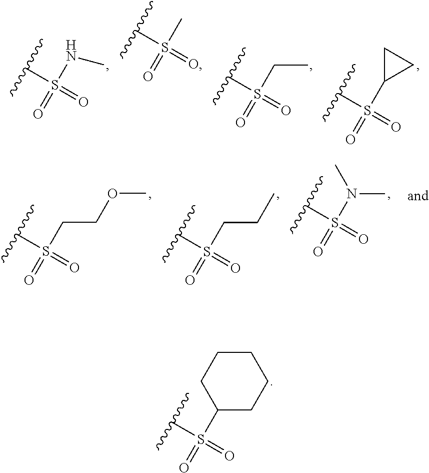

In some cases, the one or more R⁸ is selected from halogen, C₁₋₆_haloalkyl, and C₁₋₆ alkyl. In some cases, R⁷ is selected from

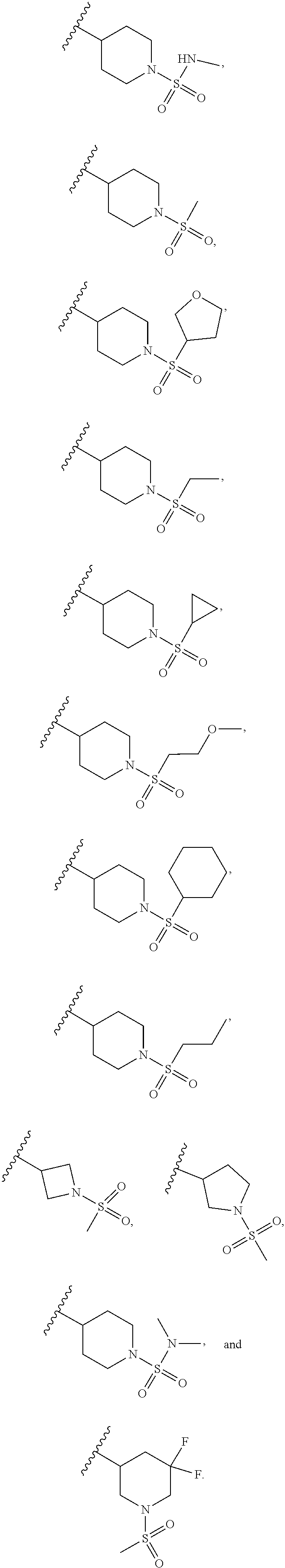

In some cases, R⁷ is selected from

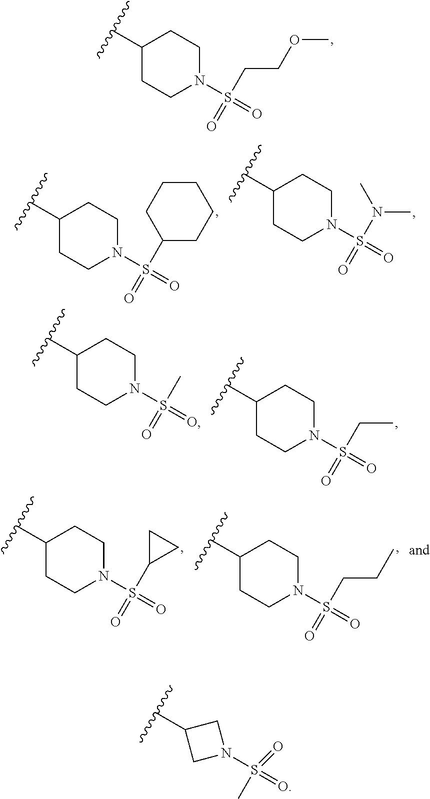

In some cases, the at least one substitutent of R⁷ is selected from —S(O)₂(R²⁰). In some cases, R²⁰ is selected from C₁₋₆ alkyl, C₃₋₆ carbocycle, and 3- to 6-membered heterocycle, each of which is optionally substituted with one or more substituents independently selected from halogen, —OH, —CN, —NH₂, C₁₋₁₀ alkyl, —C₁₋₁₀ haloalkyl, —O—C₁₋₁₀ alkyl. In some cases, the heterocycle has at least one oxygen atom. In some cases, the heterocycle has one oxygen atom and no other heteroatoms. In some cases, the heterocycle is unsubstituted. In some cases, R²⁰ is selected from C₁₋₆ alkyl, C₃₋₆ carbocycle, and 3- to 6-membered heterocycle, wherein the C₁₋₆ alkyl is optionally substituted with one or more substituents independently selected from —O—C₁₋₁₀ alkyl. In some cases, the carbocycle is unsubstituted. In some cases, R²⁰ is selected from methyl, ethyl,

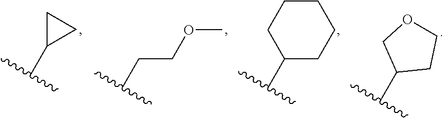

In some cases, R²⁰ is methyl. In some cases, R²⁰ is ethyl. In some cases, R²⁰ is

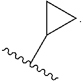 [SMILES: *C1CC1]

In some cases, R²⁰ is

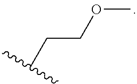 [SMILES: *CCOC]

In some cases, R²⁰ is

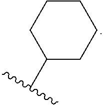 [SMILES: *C1CCCCC1]

In some cases R²⁰ is

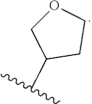 [SMILES: *C1CCOC1]

In some cases, R⁷ is selected from

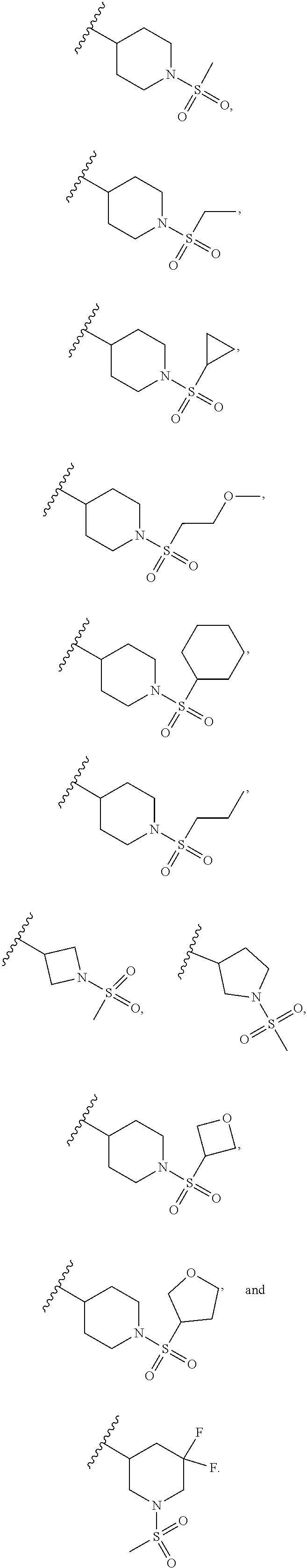

In some embodiments, for a compound or salt of Formula (I-B), or Formula (II), R⁷ is selected from

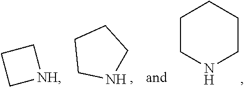

wherein each is substituted with —S(O)₂(R²⁰). In some cases, R⁷ is selected from

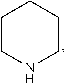

wherein the

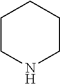

is substituted with —S(O)₂(R²⁰) and no other substituents, wherein R²⁰ is selected from C₁-C₆ alkyl. In some cases, R⁷ is selected from

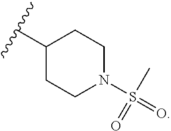 [SMILES: *C1CCN(S(C)(=O)=O)CC1]

In some cases, R⁷ is selected from

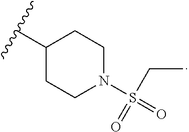 [SMILES: *C1CCN(S(=O)(=O)CC)CC1]

In some cases, R⁷ is selected from

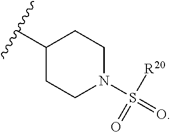

In some cases, R⁷ is selected from

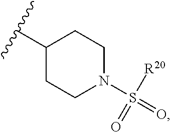

wherein R²⁰ is selected from unsubstituted C₁-C₆ alkyl. In some cases, R⁵ is hydrogen. In some cases, R⁴ is unsubstituted imidazole. In some cases, R³ is selected from hydrogen, C₁-C₆ alkyl, and C₁-C₆ haloalkyl. In some cases, R³ is selected from C₁-C₆ alkyl. In some cases, R³ is methyl. In some cases, R¹ is CH. In some cases, R² is CH.

In some embodiments, for a compound or salt of Formula (I), Formula (I-B), or Formula (II), R⁷ is selected from

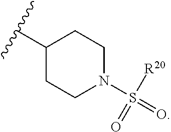

In some cases, R⁷ is selected from

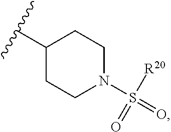

wherein R²⁰ is selected from 3- to 6-membered heterocycle, each of which is optionally substituted with one or more substituents independently selected from halogen, —OH, —CN, —NH₂, C₁₋₁₀ alkyl, —C₁₋₁₀ haloalkyl, —O—C₁₋₁₀ alkyl. In some cases, R²⁰ is selected from 5- to 6-membered heterocycle, each of which is optionally substituted with one or more substituents independently selected from halogen, —OH, —CN, —NH₂, C₁₋₁₀ alkyl, —C₁₋₁₀ haloalkyl, —O—C₁₋₁₀ alkyl. In some cases, R²⁰ is selected from

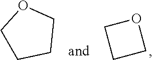

each of which is optionally substituted with one or more substituents independently selected from halogen, —OH, —CN, —NH₂, C₁₋₁₀ alkyl, —C₁₋₁₀ haloalkyl, —O—C₁₋₁₀ alkyl. In some cases, R²⁰ is selected from 5-membered heterocycle, each of which is optionally substituted with one or more substituents independently selected from halogen, —OH, —CN, —NH₂, C₁₋₁₀ alkyl, —C₁₋₁₀ haloalkyl, —O—C₁₋₁₀ alkyl. In some cases, the heterocycle has at least one oxygen atom. In some cases, the heterocycle of R²⁰ has no substituents. In some cases, the heterocycle has one oxygen atom and no other heteroatoms. In some cases, R²⁰ is

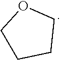

In some cases, R²⁰ is

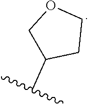 [SMILES: *C1CCOC1]

In some cases, R⁷ is

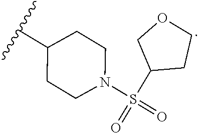 [SMILES: *C1CCN(S(=O)(=O)C2CCOC2)CC1]

In some cases, R²⁰ is

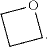

In some cases, R²⁰ is

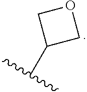 [SMILES: *C1COC1]

In some cases, R⁷ is

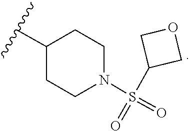 [SMILES: *C1CCN(S(=O)(=O)C2COC2)CC1]

In some embodiments, for a compound or salt of Formula (I) or Formula (I-A), R³ is selected from —O—C₁-C₆ alkyl, and C₁-C₆ alkyl, wherein the C₁-C₆ alkyl is optionally substituted with one or more substituents independently selected from halogen. In some cases, R³ is selected from —CF₃, —CH₃, and —OCH₃. In some cases, R³ is —CH₃. In some cases, R¹ is CH. In some cases, R² is CH. In some cases, R¹ is N. In some cases, R² is N.

In some embodiments, Formula (I) is represented by Formula (I-C)

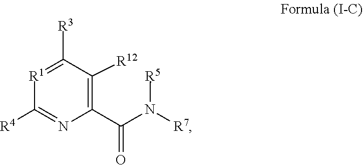

or a pharmaceutically acceptable salt thereof, wherein:
R¹² is selected from halogen, —NH₂, C₁-C₆ alkyl, —CF₃, and —O—C₁₋₁₀ alkyl; and
R⁷ is selected from an optionally substituted saturated 4- to 8-membered heterocycle, which is optionally substituted with one or more R⁸.

In some embodiments, for a compound or salt of Formula (I) or Formula (I-C), R¹² is selected from C₁-C₆ alkyl. In some cases, R¹² is methyl.

In some embodiments, for a compound or salt of Formula (I) or Formula (I-C), R¹ is CH. In some cases, R¹ is N.

In some embodiments, for a compound or salt of Formula (I) or Formula (I-C), R³ is selected from hydrogen, —O—C₁-C₆ alkyl, and C₁-C₆ alkyl, wherein the C₁-C₆ alkyl is optionally substituted with one or more substituents independently selected from halogen. In some cases, R³ is selected from hydrogen —CF₃, —CH₃, and —OCH₃. In some cases, R³ is selected from hydrogen.

In some embodiments, for a compound or salt of Formula (I) or Formula (I-C), R⁷ is selected from an optionally substituted saturated 4- to 8-membered heterocycle. In some cases, R⁷ is selected from a saturated 4- to 8-membered heterocycle, wherein the saturated 4- to 8-membered heterocycle is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the saturated 4- to 8-membered heterocycle is further optionally substituted with one or more R⁸. In some cases, the saturated heterocycle of R⁷ is selected from

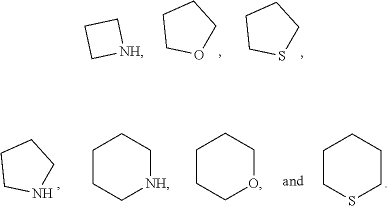

In some cases, the saturated heterocycle of R⁷ is selected from

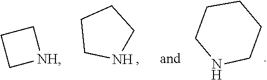

In some cases, the saturated heterocycle of R⁷ is selected from

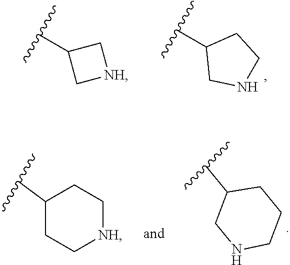 [SMILES: *C1CNC1 ; *C1CCNC1 ; *C1CCNCC1 ; *C1CCCNC1]

In some cases, the at least one substitutent of R⁷ is selected from —S(O)₂(R²⁰), and —S(O)₂(NR²⁰₂). In some cases, the at least one substituent of R⁷ is selected from

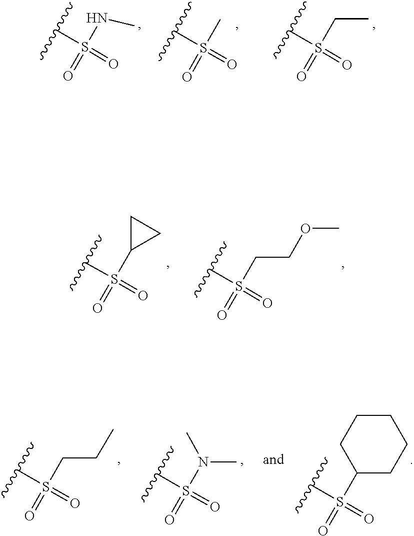

In some cases, the one or more R⁸ is selected from halogen, C₁₋₆ haloalkyl, and C₁₋₆ alkyl. In some cases, R⁷ is selected from

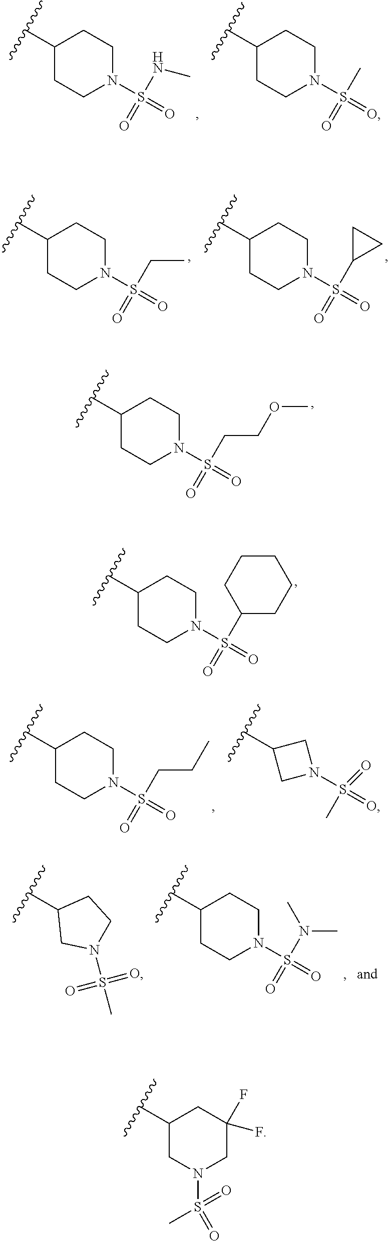

In some cases, R⁷ is selected from

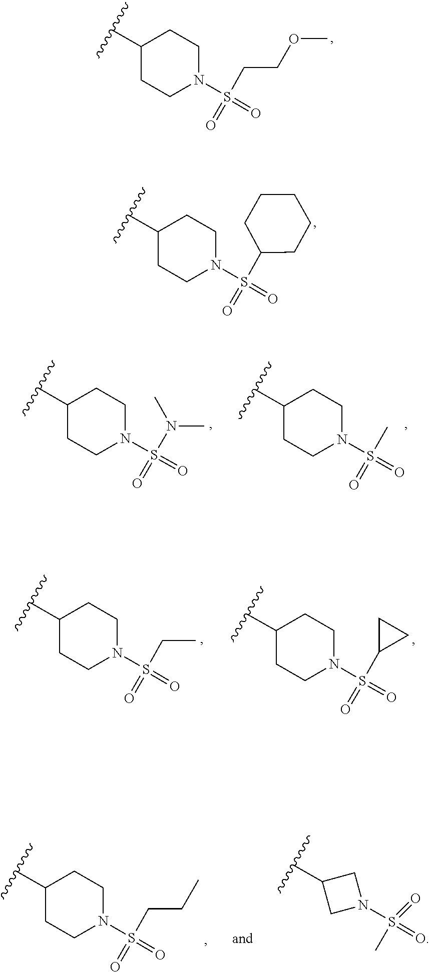

In some cases, the at least one substitutent of R⁷ is selected from —S(O)₂(R²⁰). In some cases R⁷ is selected from

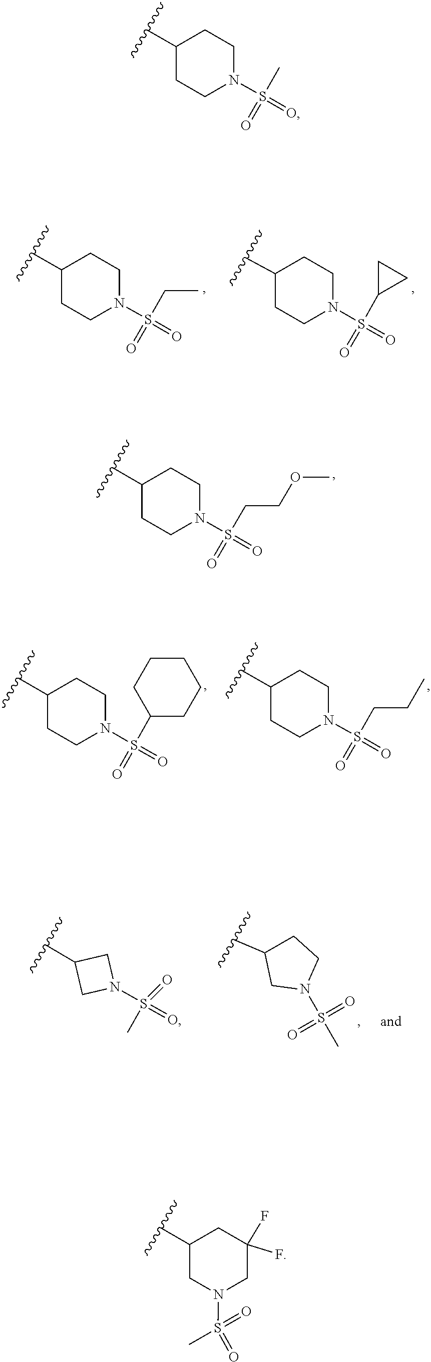

In some embodiments, for a compound or salt of Formula (I) or Formula (I-C), R⁷ is selected from a C₃₋₅ cycloalkyl, and C₇₋₁₀ cycloalkyl, each of which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein each is further optionally substituted with one or more R⁸. In some cases, R⁷ is selected from a C₃₋₅ cycloalkyl, and C₇₋₁₀ cycloalkyl, each of which is substituted with at least one substitutent selected from —S(O)₂(R²⁰) and —S(O)₂(NR²⁰₂).

In some embodiments, for a compound or salt of Formula (I) or Formula (I-C), R⁷ is selected from a C₆ cycloalkyl substituted with at least one substitutent selected from —S(O)₂(R²⁰), —S(O)(R²⁰), —S(O)₂(NR²⁰₂), —S(O)(NR²¹)R²⁰; and wherein the C₆ cycloalkyl is further optionally substituted with one or more R^{BA}. In some cases, R⁷ is selected from a C₆ cycloalkyl substituted with at least one substitutent selected from —S(O)₂(R²⁰), and —S(O)₂(NR²⁰₂).

In some embodiments, for a compound or salt of Formula (I) or Formula (I-C), R⁷ is selected from an optionally substituted saturated 4- to 6-membered heterocycle. In some cases, R⁷ is selected from an optionally substituted saturated 5- to 6-membered heterocycle. In some cases, R⁷ is selected from an optionally substituted saturated 5- to 6-membered heterocycle, wherein the saturated 5- to 6-membered heterocycle has at least one heteroatom selected from oxygen, nitrogen, and sulfur. In some cases, R⁷ is selected from an optionally substituted saturated 4- to 6-membered heterocycle, wherein the saturated 4- to 6-membered heterocycle has at least one heteroatom selected from oxygen, nitrogen, and sulfur. In some cases, R⁷ is selected from

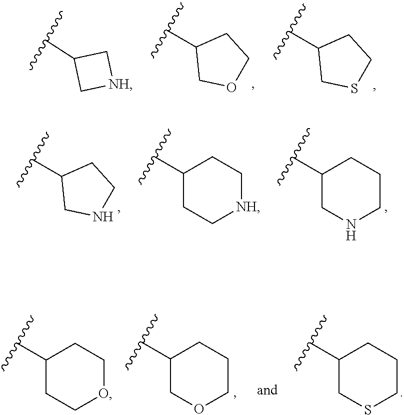

wherein each is optionally substituted with one or more substituents independently selected from R⁸. In some cases, each R⁸ is independently selected from halogen, —OR²⁰, ═O, C₁₋₆ hydroxyalkyl, C₁₋₆haloalkyl, C₁₋₆ alkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁸ is selected from halogen, ═O, C₁₋₆ hydroxyalkyl, C₁ haloalkyl, C₁₋₆ alkyl, —C(O)R²⁰, —S(O)₂(R²⁰), —S(O)(R²⁰), —S(O)₂(NR²⁰₂), —S(O)(NR²¹)R²⁰, and —S(O)(NR²¹)N(R²¹)₂.

In some embodiments, for a compound or salt of Formula (I) or Formula (I-C), R²⁰ is independently selected at each occurrence from hydrogen, C₁₋₆ alkyl, and C₃₋₁₂ carbocycle.

In some embodiments, for a compound or salt of Formula (I) or Formula (I-C), R²¹ is independently selected at each occurrence from hydrogen, C₁₋₆ alkyl, and C₃₋₁₂ carbocycle.

In some embodiments, for a compound or salt of Formula (I) or Formula (I-C), R⁷ is selected from

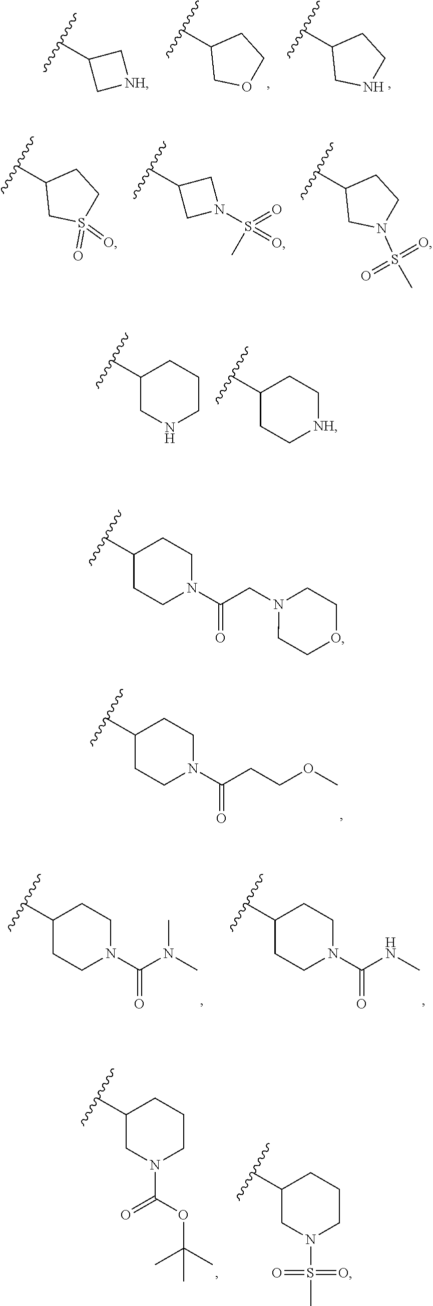

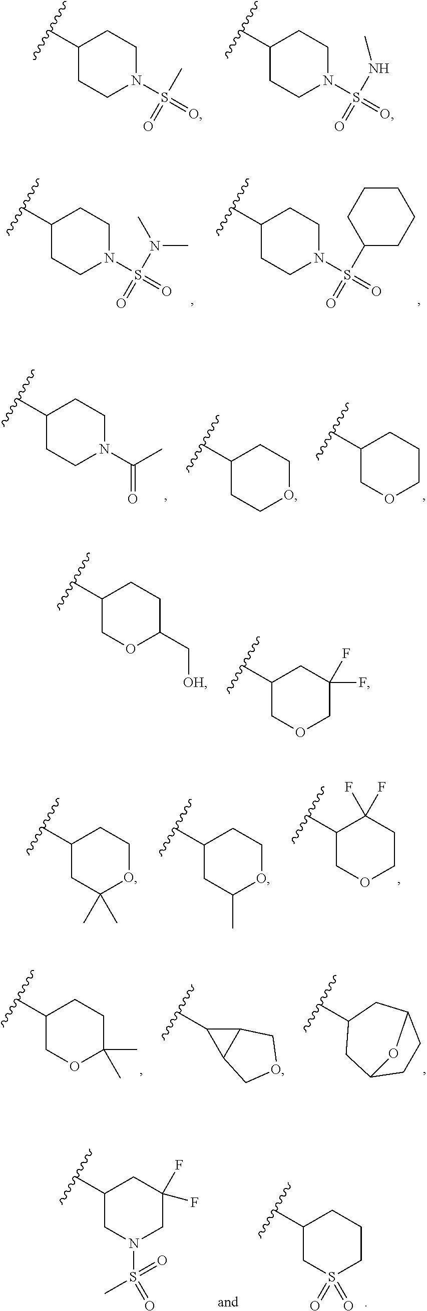

In some cases, R⁷ is selected from

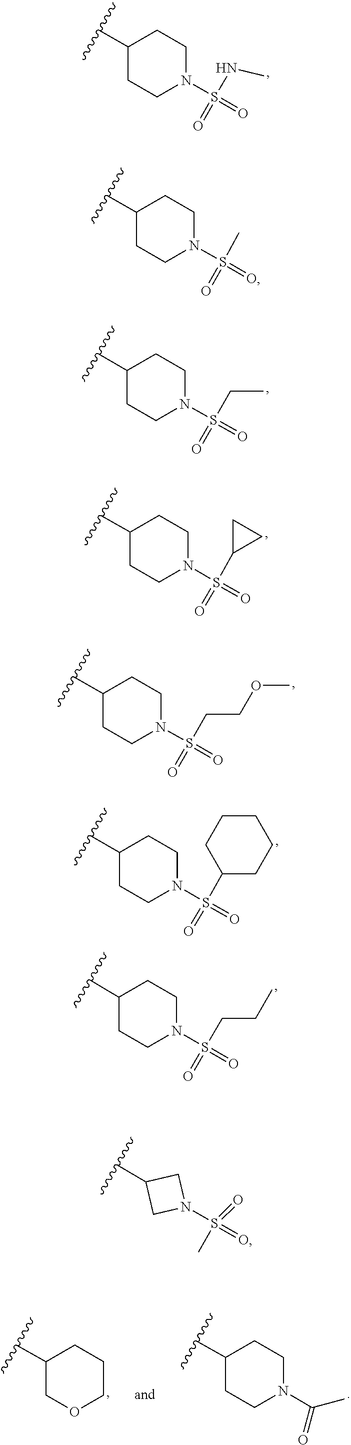

In some embodiments, for a compound or salt of Formula (I) or Formula (I-C), R⁷ is selected from

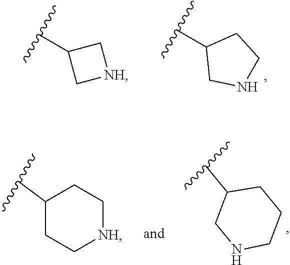 [SMILES: *C1CNC1 ; *C1CCNC1 ; *C1CCNCC1 ; *C1CCCNC1]

wherein each is optionally substituted with one or more substituents independently selected from R⁸. In some cases, R⁷ is selected from

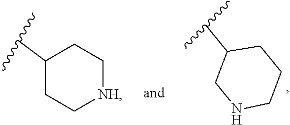 [SMILES: *C1CCNCC1 ; *C1CCCNC1]

wherein each is optionally substituted with one or more substituents independently selected from R⁸. In some cases, R⁷ is

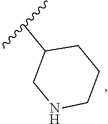 [SMILES: *C1CCCNC1]

which is optionally substituted with one or more substituents independently selected from R⁸. In some cases, R⁷ is selected from

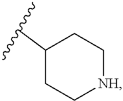 [SMILES: *C1CCNCC1]

which is optionally substituted with one or more substituents independently selected from R⁸. In some cases, each R⁸ is selected from C₁₋₆ alkyl, ═O, and saturated 4- to 8-membered heterocycle. In some cases, R⁷ is selected from

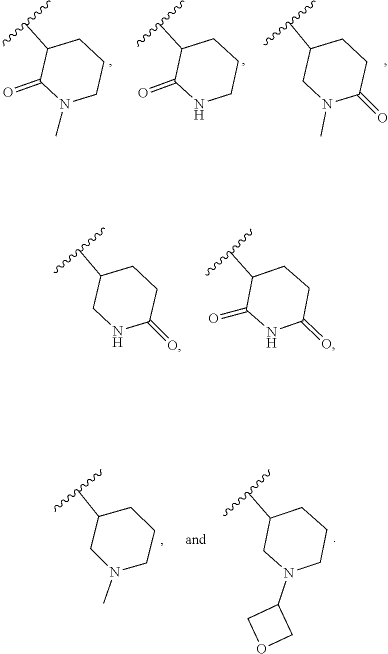

In some cases, each R⁸ is selected from C₁₋₆ alkyl, and ═O. In some cases, R⁷ is selected from

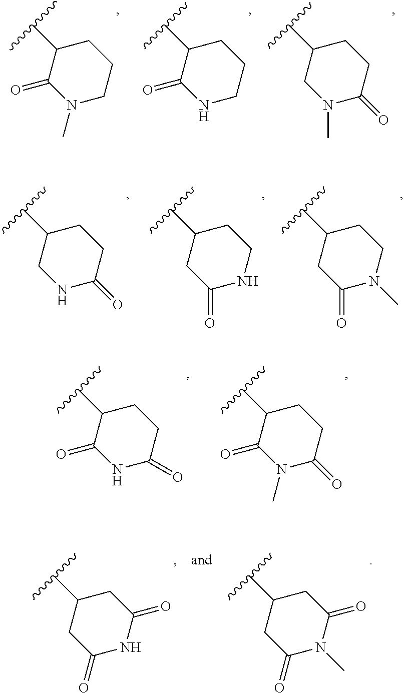

In some embodiments, for a compound or salt of Formula (I) or Formula (I-C), R⁷ is selected from an optionally substituted saturated 7- to 8-membered spiro heterocycle. In some cases, R⁷ is selected from

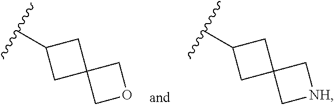 [SMILES: *C1CC2(COC2)C1 ; *C1CC2(CNC2)C1]

each of which is optionally substituted with one or more substituents independently selected from R⁸. In some cases, R⁷ is selected from

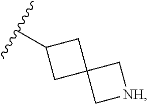 [SMILES: *C1CC2(CNC2)C1]

which is optionally substituted with one or more substituents independently selected from R⁸. In some cases, each R⁸ is independently selected from halogen, —OR²⁰, —SR²⁰, —N(R²¹)₂, —NO₂, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, —C(O)R²⁰, —C(O)OR²⁰, —C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, each R⁸ is independently selected from C₁₋₆ alkyl, —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁸ is independently selected from —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁷ is

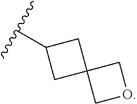 [SMILES: *C1CC2(COC2)C1]

In some embodiments, for a compound or salt of Formula (I) or Formula (I-C), R⁷ is selected from an optionally substituted saturated 4- to 8-membered heterocycle, wherein the 4- to 8-membered heterocycle contains at least one oxygen atom. In some cases, the 4- to 8-membered heterocycle contains one oxygen atom and no other heteroatoms. In some cases, the heterocycle is selected from

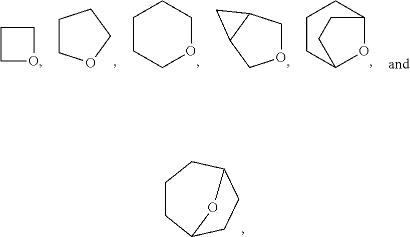

each of which is optionally substituted with one or more substituents independently selected from R⁸. In some cases, the heterocycle is selected from

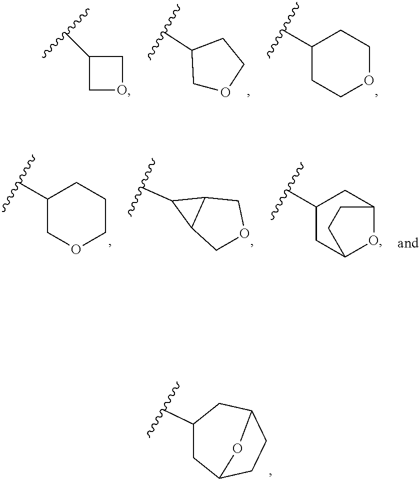

each of which is optionally substituted with one or more substituents independently selected from R⁸. In some cases, wherein each R⁸ is independently selected from halogen, C₁₋₆ alkyl, and C₁₋₆ hydroxyalkyl. In some cases, R⁷ is selected from

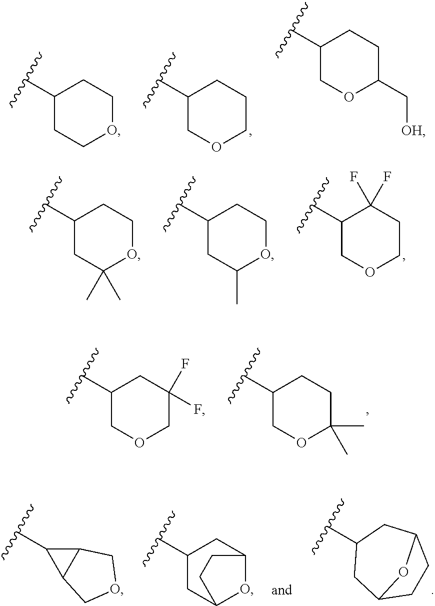

In some embodiments, for a compound or salt of Formula (I) or Formula (I-C), the heterocycle of R⁷ is substituted with at least one or more substituents independently selected from R⁸. In some cases, R⁷ is selected from substituted C₆ cycloalkyl and optionally substituted saturated 6-membered heterocycle.

In some embodiments, for a compound or salt of Formula (I) or Formula (I-C), each R^{8A} is independently selected from halogen and each R⁸ is independently selected from —C(O)N(R²¹)₂, —S(O)₂(R²⁰), and —S(O)₂(NR²⁰₂).

In some embodiments, for a compound or salt of Formula (I) or Formula (I-C), R⁷ is selected from

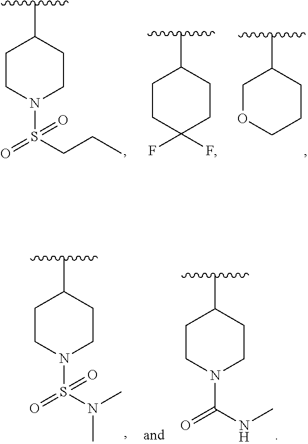

In some embodiments, for a compound or salt of Formula (I) or Formula (I-C), R⁷ is selected from a substituted C₆ cycloalkyl. In some cases, R⁷ is selected from C₆ cycloalkyl substituted with one or more R^{8A}, wherein R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)(NR²¹)R²⁰, and —S(O)(NR²¹)N(R²¹)₂. In some cases, R^{8A} is independently selected at each occurrence from halogen, C₁₋₆ haloalkyl, —C(O)R²⁰, —S(O)₂(R²⁰), —S(O)(R²⁰), —S(O)(NR²¹)R²⁰, and —S(O)(NR²¹)N(R²¹)₂. In some cases, R^{8A} is independently selected at each occurrence from halogen, —C(O)R²⁰, and —S(O)₂(R²⁰). In some cases, R⁷ is selected from

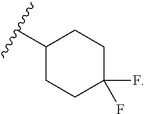 [SMILES: *C1CCC(F)(F)CC1]

In some embodiments, for a compound or salt of Formula (I) or Formula (I-C), R⁷ is selected from C₃₋₅ cycloalkyl, and C₇₋₈ cycloalkyl, each of which is optionally substituted with one or more R⁸. In some cases, R⁷ is selected from

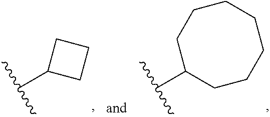 [SMILES: *C1CCC1 ; *C1CCCCCCC1]

each of which is optionally substituted with one or more R⁸. In some cases, R⁷ is selected from

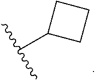 [SMILES: *C1CCC1]

In some embodiments, for a compound or salt of Formula (I) or Formula (I-C), R⁷ is selected from C₈₋₁₀ cycloalkyl, which is optionally substituted with one or more R⁸. In some cases, R⁷ is

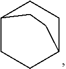

which is optionally substituted with one or more R⁸. In some cases, R⁷ is

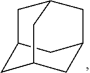

which is optionally substituted with one or more R⁸. In some cases, each R⁸ is independently selected at each occurrence from —OH, and —O—C₁₋₆ alkyl.

In some embodiments, for a compound or salt of Formula (I) or Formula (I-C), R³ is selected from hydrogen, halogen, —CN, —OR¹⁶, C₁-C₆ alkyl, and 3- to 12-membered heterocycle, wherein the C₁-C₆ alkyl, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle. In some cases, R³ is selected from hydrogen, —OR¹⁶, —SO₂R¹⁶, optionally substituted C₁-C₆ alkyl, and 3- to 12-membered heterocycle, wherein the C₁-C₆ alkyl and 3- to 12-membered heterocycle are each optionally substituted with one or more substituents independently selected from halogen, —OH, —CN, —NO₂, —NH₂, —NHC₁₋₁₀ alkyl, C₁-C₆ alkyl, —N(C₁₋₁₀ alkyl)₂, —C₁₋₁₀ haloalkyl, and —O—C₁₋₁₀ alkyl. In some cases, R³ is selected from hydrogen, —OR¹⁶, C₁-C₆ alkyl, and 5- to 6-membered heterocycle, wherein the 5- to 6-membered heterocycle is optionally substituted with one or more substituents independently selected from C₁-C₆ alkyl. In some cases, R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl. In some cases, R¹⁶ is selected from hydrogen; C₁₋₆ alkyl, and C₃₋₁₂ carbocycle. In some cases, R⁵ is hydrogen.

In some embodiments, for a compound or salt of Formula (I) or Formula (I-C), R⁴ is selected from imidazole, thiazole, and oxazole, each of which is optionally substituted with one or more R⁹. In some cases, R⁴ is selected from imidazole and thiazole, each of which is optionally substituted with one or more R⁹. In some cases, R⁴ is selected from imidazole, each of which is optionally substituted with one or more R⁹. In some cases, R⁴ is

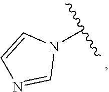 [SMILES: *n1ccnc1]

which is optionally substituted with one or more R⁹. In some cases, R⁴ is

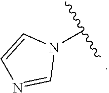 [SMILES: *n1ccnc1]

In an aspect, the present disclosure provides a compound of Formula (II):

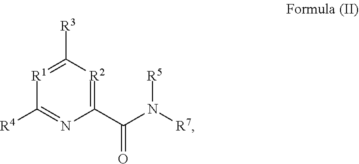

or a pharmaceutically acceptable salt thereof, wherein:
R¹ is selected from N and CR¹¹;
R² is selected from N and CR¹²;
R³ is selected from hydrogen, halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is imidazole, wherein the imidazole is optionally substituted with one or more R⁹;
R⁵ is selected from hydrogen and C₁-C₆ alkyl;
R⁷ is selected from an 4- to 8-membered heterocycle and C₃₋₁₂ carbocycle, each of which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the 4- to 8-membered heterocycle and C₃₋₁₂ carbocycle are each further optionally substituted with one or more R⁸;
each R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

In some embodiments, for a compound or salt of Formula (II), R⁷ is selected from a 4-to 8-membered heterocycle, which is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂.

In some embodiments, for a compound or salt of Formula (II), R⁷ is selected from a saturated 4- to 8-membered heterocycle, wherein the saturated 4- to 8-membered heterocycle is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁷ is selected from a saturated 4- to 8-membered heterocycle, wherein the saturated 4- to 8-membered heterocycle is substituted with —S(O)₂(R²⁰). In some cases, the heterocycle of R⁷ is selected from

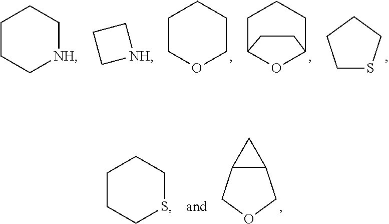

each of which is substituted. In some cases, the heterocycle of R⁷ is selected from

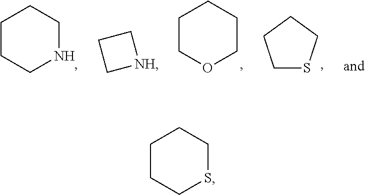

each of which is substituted. In some cases, the heterocycle of R⁷ is selected from

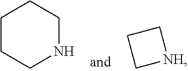

each of which is substituted. In some cases, the heterocycle of R⁷ is selected from

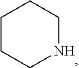

which is substituted. In some cases, R⁷ is selected from

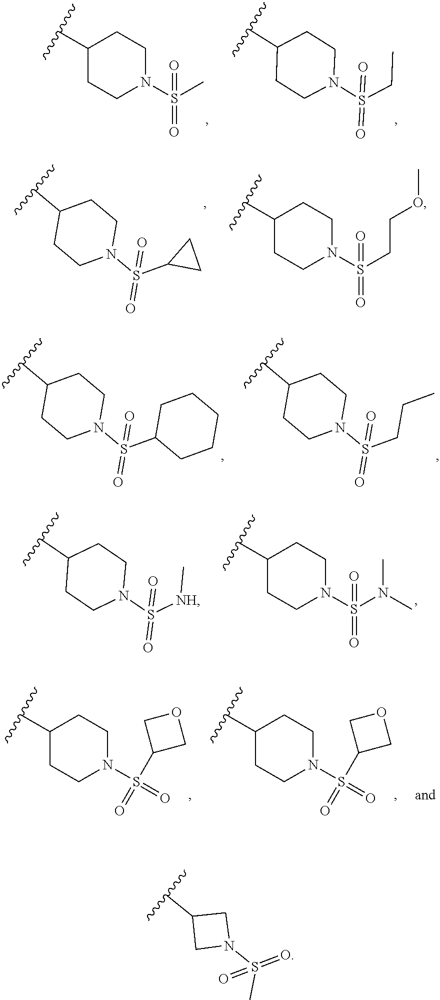

In some embodiments, for a compound or salt of Formula (II), R⁷ is selected from a phenyl.

In some embodiments, for a compound or salt of Formula (II), R⁷ is selected from an optionally substituted saturated C₃₋₈ carbocycle. In some cases, R⁷ is selected from

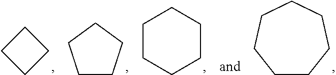

each of which is substituted.

In some embodiments, for a compound or salt of Formula (II), R⁷ is substituted with at least one substitutent selected from —S(O)₂(R²⁰), —S(O)(R²⁰), —S(O)₂(NR²⁰₂), —S(O)(NR²¹)R²⁰, and —S(O)(NR²¹)N(R²¹)₂. In some cases, R⁷ is substituted with at least one substitutent selected from —S(O)₂(R²⁰) and —S(O)₂(NR²⁰₂). In some cases, R⁷ is substituted with at least one substitutent selected from —S(O)₂(R²⁰).

In some embodiments, for a compound or salt of Formula (II), each R⁸ is independently selected at each occurrence from halogen, —OR²⁰, —N(R²¹)₂, ═O, —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl. In some cases, each R⁸ is independently selected at each occurrence from C₁₋₆haloalkyl and C₁₋₆ alkyl.

In some embodiments, for a compound or salt of Formula (II), R⁴ is

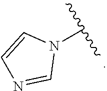 [SMILES: *n1ccnc1]

In some embodiments, for a compound or salt of Formula (II), R¹ is N. in some cases, R¹ is CH.

In some embodiments, for a compound or salt of Formula (II), R² is N. In some cases, R² is CH.

In some embodiments, for a compound or salt of Formula (II), R³ is hydrogen. In some cases, R³ is selected from halogen, —CN, —OR¹⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle. In some cases, R³ is selected from C₁-C₆ alkyl and C₁-C₆ halolkyl. In some cases, R³ is selected from C₁-C₆ alkyl. In some cases, R³ is selected from C₁-C₆ halolkyl.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

When stereochemistry is not specified, molecules with stereocenters described herein include isomers, such as enantiomers and diastereomers, mixtures of enantiomers, including racemates, mixtures of diastereomers, and other mixtures thereof, to the extent they can be made by one of ordinary skill in the art by routine experimentation. In certain embodiments, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration.

In certain embodiments, compositions of the disclosure may comprise two or more enantiomers or diatereomers of a compound wherein a single enantiomer or diastereomer accounts for at least about 70% by weight, at least about 80% by weight, at least about 90% by weight, at least about 98% by weight, or at least about 99% by weight or more of the total weight of all stereoisomers. Methods of producing substantially pure enantiomers are well known to those of skill in the art. For example, a single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller (1975) J Chromatogr., 113(3): 283-302). Racemic mixtures of chiral compounds can be separated and isolated by any suitable method, including, but not limited to: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. Another approach for separation of the enantiomers is to use a Diacel chiral column and elution using an organic mobile phase such as done by Chiral Technologies (www.chiraltech.com) on a fee for service basis.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

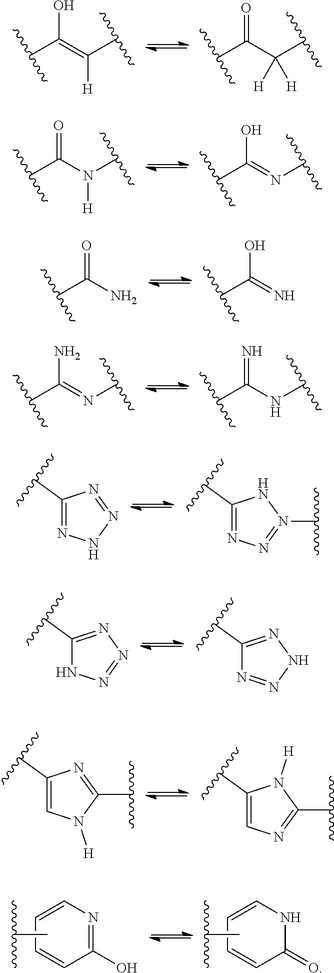

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, 12N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

A compound or salt of any one of the Formulas or sub Formulas described herein (e.g., Formula (I), Formula (I-A), Formula (I-B), Formula (I-C), or Formula (II)) may be formulated in any suitable pharmaceutical formulation. A pharmaceutical formulation of the present disclosure typically contains an active ingredient (e.g., compound or salt of any one of the Formulas described herein) and one or more pharmaceutically acceptable excipients or carriers, including but not limited to: inert solid diluents and fillers, diluents, sterile aqueous solution and various organic solvents, permeation enhancers, antioxidents, solubilizers, and adjuvants.

In certain embodiments, a compound or salt of any one of the Formulas or sub Formulas described herein is formulated with an agent that inhibits degradation of the compound or salt. In certain embodiments, the compound or salt is formulated with one or more antioxidants. Acceptable antioxidants include, but are not limited to, citric acid, d,I-α-tocopherol, BHA, BHT, monothioglycerol, ascorbyl palmitate, ascorbic acid, and propyl gallate. In certain embodiments, the formulation contains from 0.1 to 30%, from 0.5 to 25%, from 1 to 20%, from 5 to 15%, or from 7 to 12% (wt/wt) CCI-779, from 0.5 to 50%, from 1 to 40%, from 5 to 35%, from 10 to 25%, or from 15 to 20% (wt/wt) water soluble polymer, from 0.5 to 10%, 1 to 8%, or 3 to 5% (wt/wt) surfactant, and from 0.001% to 1%, 0.01% to 1%, or 0.10% to 0.5% (wt/wt) antioxidant. In certain embodiments, the antioxidants of the formulations of this invention will be used in concentrations ranging from 0.001% to 3% wt/wt.

In certain embodiments, a compound or salt of any one of the Formulas or sub Formulas described herein is formulated with a pH modifying agent to maintain a pH of about 4 to about 6. Acceptable pH modifying agents include, but are not limited to citric acid, sodium citrate, dilute HCl, and other mild acids or bases capable of buffering a solution containing a compound or a salt of the disclosure to a pH in the range of about 4 to about 6.

In certain embodiments, a compound or salt of any one of the Formulas or sub Formulas described herein is formulated with a chelating agent or other material capable of binding metal ions, such as ethylene diamine tetra acetic acid (EDTA) and its salts are capable of enhancing the stability of a compound or salt of any one of the Formulas described herein.

Pharmaceutical formulations may be provided in any suitable form, which may depend on the route of administration. In some embodiments, the pharmaceutical composition disclosed herein can be formulated in dosage form for administration to a subject. In some embodiments, the pharmaceutical composition is formulated for oral, intravenous, intraarterial, aerosol, parenteral, buccal, topical, transdermal, rectal, intramuscular, subcutaneous, intraosseous, intranasal, intrapulmonary, transmucosal, inhalation, and/or intraperitoneal administration. In some embodiments, the dosage form is formulated for oral administration. For example, the pharmaceutical composition can be formulated in the form of a pill, a tablet, a capsule, an inhaler, a liquid suspension, a liquid emulsion, a gel, or a powder. In some embodiments, the pharmaceutical composition can be formulated as a unit dosage in liquid, gel, semi-liquid, semi-solid, or solid form.

The amount of a compound or salt of any one of the Formulas or sub Formulas described herein will be dependent on the mammal being treated, the severity of the disorder or condition, the rate of administration, the disposition of the compound or salt of any one of the Formulas or sub Formulas described herein and the discretion of the prescribing physician.

In some embodiments, pharmaceutically acceptable carriers of a compound or salt of any one of the Formulas or sub Formulas described herein can include a physiologically acceptable compound that is an antioxidant.

In some embodiments, the disclosure provides a pharmaceutical composition for oral administration containing at least one compound or salt of any one of the Formulas or sub Formulas described herein and a pharmaceutical excipient suitable for oral administration. The composition may be in the form of a solid, liquid, gel, semi-liquid, or semi-solid. In some embodiments, the composition further comprises a second agent.

Pharmaceutical compositions of the disclosure suitable for oral administration can be presented as discrete dosage forms, such as hard or soft capsules, cachets, troches, lozenges, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion, or dispersible powders or granules, or syrups or elixirs. Such dosage forms can be prepared by any of the methods of pharmacy, which typically include the step of bringing the active ingredient(s) into association with the carrier. In general, the composition are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient(s) in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound or salt of any one of the Formulas or sub Formulas described herein moistened with an inert liquid diluent.

In some embodiments, the disclosure provides a pharmaceutical composition for injection containing a compound or salt of any one of the Formulas described herein and a pharmaceutical excipient suitable for injection. Components and amounts of agents in the composition are as described herein.

In certain embodiments, the compound or salt of any one of the Formulas or sub Formulas described herein may be formulated for injection as aqueous or oil suspensions, emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Pharmaceutical compositions may also be prepared from a compound or salt of any one of the Formulas or sub Formulas described herein and one or more pharmaceutically acceptable excipients suitable for transdermal, inhalative, sublingual, buccal, rectal, intraosseous, intraocular, intranasal, epidural, or intraspinal administration. Preparations for such pharmaceutical composition are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999).

The disclosure also provides kits. The kits may include a compound or salt of any one of the Formulas or sub Formulas described herein and one or more additional agents in suitable packaging with written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, a compound or salt of any one of the Formulas or sub Formulas described herein and the agent are provided as separate compositions in separate containers within the kit. In some embodiments, a compound or salt of any one of the Formulas or sub Formulas described herein and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Therapeutic Applications

In one aspect, the present disclosure provides a method of inhbiting CD38 comprising administering a compound or salt of Formula (I), Formula (I-A), Formula (I-B), Formula (I-C), or Formula (II). In some cases, the method comprises inhibiting CD38 by administering a pharmaceutical composition comprising a compound or salt of Formula (I), Formula (I-A), Formula (I-B), Formula (I-C), or Formula (II), and a pharmaceutically acceptable excipient.

In some cases, a compound or salt of Formula (I), Formula (I-A), Formula (I-B), Formula (I-C), or Formula (II), is administered to a subject in need thereof to inhibit CD38 and thereby raise NAD+ levels. In some cases, raising NAD+ levels may be useful in treating diseases or conditions indicated to benefit from NAD+ including mitochondrial-related diseases or disorders.

In some embodiments, a compound or salt of Formula (I), Formula (I-A), Formula (I-B), Formula (I-C), or Formula (II), is administered to a subject in need thereof for the treatment and/or prevention of neurodegenerative disease, type I diabetes, insulin resistance, Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra, Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, cystic fibrosis, pancreatitis, idiopathic pulmonary fibrosis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia—telangiectasia, rheumatoid arthritis, lupus, alcohol intolerance, hyperphosphatemia, acute lung injury, and ARDS.

In some cases, a pharmaceutical composition comprising a compound or salt of Formula (I), Formula (I-A), Formula (I-B), Formula (I-C), or Formula (II), and a pharmaceutically acceptable excipient is adminstered to a subject in need thereof for the treatment of a disease or disorder described herein.

### EXAMPLES

The following examples are offered to illustrate, but not to limit the claimed invention. It will be recognized that these preparation methods are illustrative and not limiting. Using the teaching provided herein, numerous other methods of producing the compounds described herein will be available to one of skill in the art.

### Example 1: General Synthetic Schemes

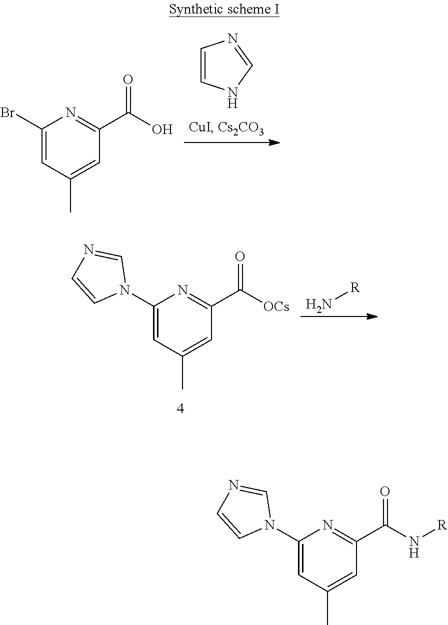

First the substituted nicotinic acid is coupled with imidazole using Ullman conditions to generate the desired N-functionalised compound 4. This carboxylate salt can then be coupled with a range of amines using multiple standard amide coupling conditions to generate the final synthetic compounds.

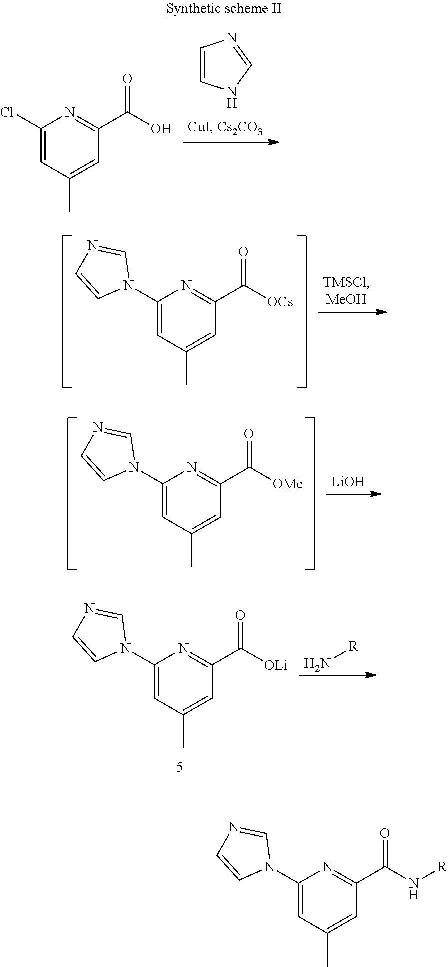

First the substituted nicotinic acid is coupled with imidazole using Ullman conditions to generate the desired N-functionalised compound which was carried directly into the next step as the crude cesium salt. This salt was reacted with acidic methanol generated from mixing TMSCl and methanol to generate the desired methyl ester which was used directly in the next step. The methyl ester is saponified using lithium hydroxide and corresponding lithium salt 5 was isolated. This carboxylate salt can then be coupled with a range of amines using standard amide coupling conditions to generate the final synthetic compounds.

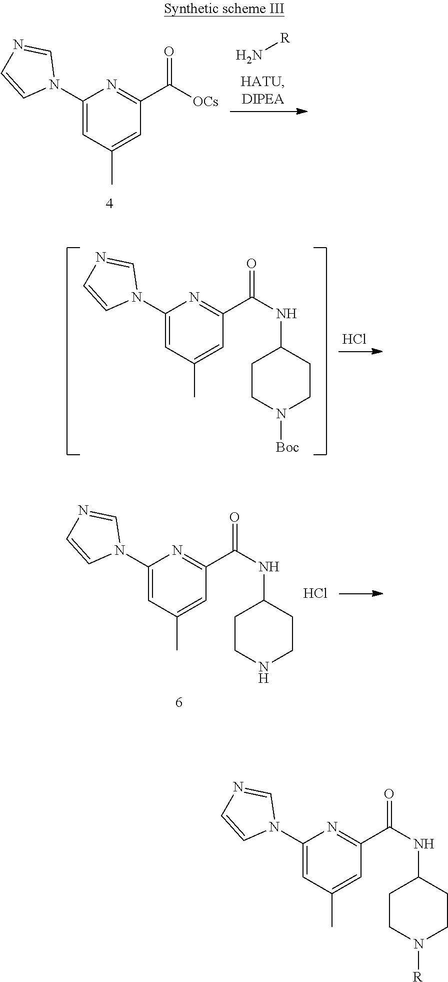

The previously synthesised intermediate carboxylate salt 4 can be coupled with the corresponding Boc-protected amine using HATU/DIPEA amide coupling conditions to generate the amide intermediate. This can then be isolated crude and deprotected with acid generating HCl salt 6. This can be coupled with a range of activated electrophiles to generate the final synthetic compounds.

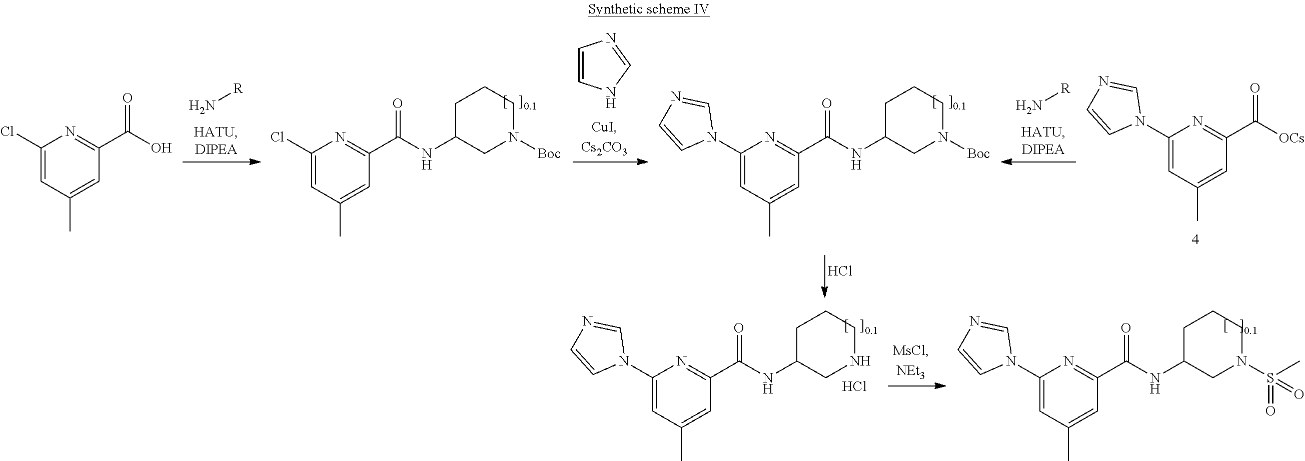

First the substituted nicotinic acid is coupled with the corresponding Boc amine saturated heterocycle using standard HATU/DIPEA amide coupling conditions. This is then reacted with imidazole under Ullman conditions to generate the desired Boc amine imidazole substituted intermediate. Alternatively, the already substituted imidazole compound 4 can be coupled with the corresponding amine to generate the same intermediate using amide coupling conditions. The Boc group can then be removed with HCl in dioxane isolating either the mono or di-HCl salts. This is then sulfonylated with mesyl chloride using triethylamine as a base to generate the final synthetic compounds.

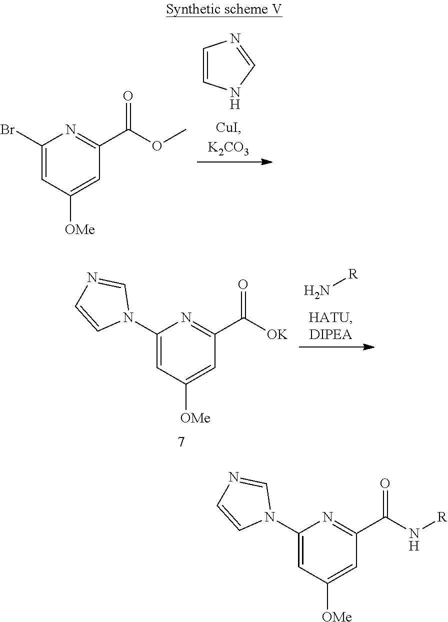

First the substituted picolinate ester is coupled with imidazole using Ullman conditions generating the desired N-functionalised compound 7 as a potassium carboxylate salt. This carboxylate can then be coupled with a range of amines using HATU/DIPEA amide coupling conditions to generate the final synthetic compounds.

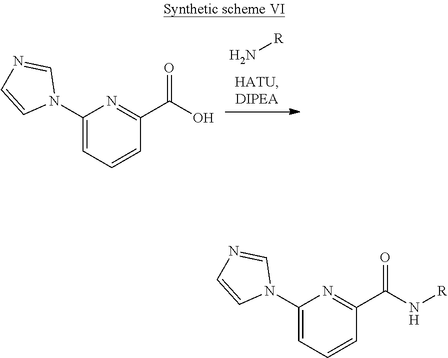

The substituted nicotinic acid is coupled with a range of amines using HATU/DIPEA amide coupling conditions to generate the final synthetic compounds.

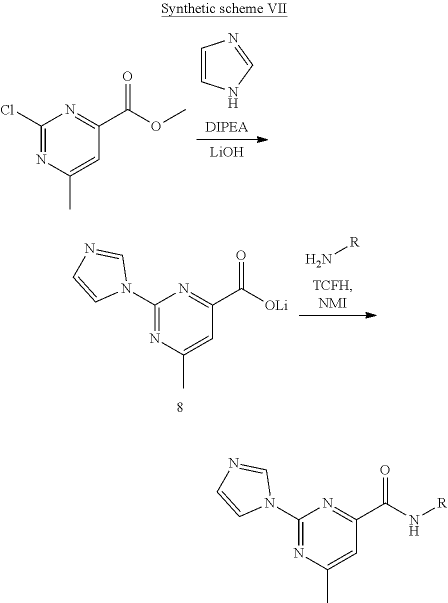

First the substituted chloro-pyrimidine is reacted with imidazole using S_{N}Ar conditions and the crude mixture of acid and ester then hydrolysed with lithium hydroxide generating the desired N-functionalised compound as lithium carboxylate salt 8. This carboxylate can then be coupled with a range of amines using NMI/TCFH amide coupling conditions to generate the final synthetic compounds.

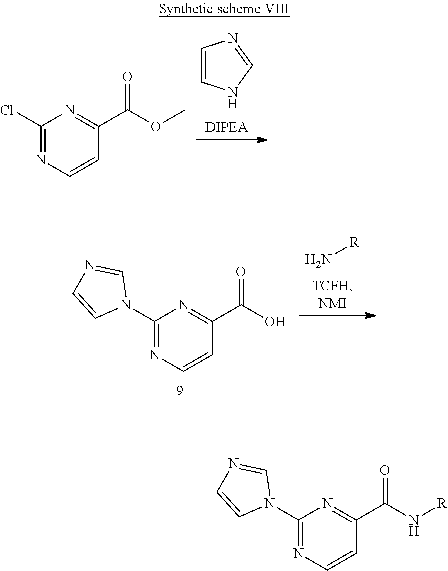

First the substituted chloro-pyrimidine is reacted with imidazole using S_{N}Ar conditions and desired N-functionalised compound 9 is isolated as an acid. Acid 9 can then be coupled with a range of amines using NMI/TCFH amide coupling conditions to generate the final synthetic compounds.

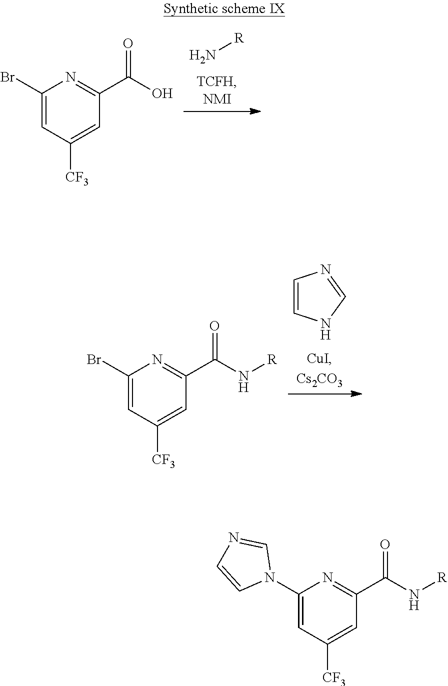

First the substituted nicotinic carboxylic acid is coupled with the corresponding amines using NMI/TCFH amide coupling conditions to generate the amide intermediates. These can then be coupled with imidazole using Ullman conditions to generate the final synthetic compounds.

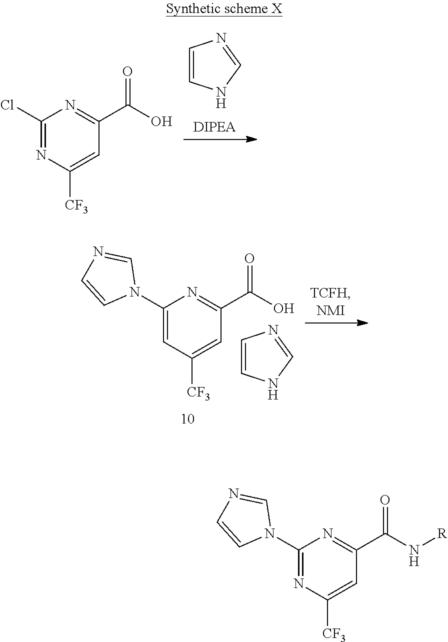

First the substituted chloro-pyrimidine reacts with imidazole under S_{N}Ar conditions, and desired acid 10 is isolated as a 1:1 salt with imidazole. This carboxylate salt can then be coupled with a range of amines using NMI/TCFH amide coupling conditions to generate the final synthetic compounds.

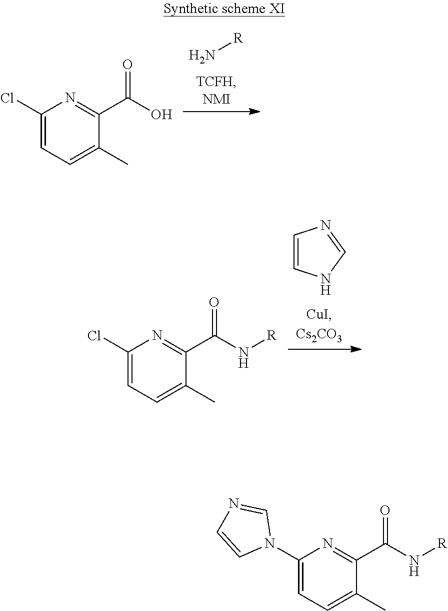

First the substituted nicotinic carboxylic acid is coupled with the corresponding amines using NMI/TCFH amide coupling conditions to generate the amide intermediates. These can then be coupled with imidazole using Ullman conditions to generate the final synthetic compounds.

### Example 2: General Analysis Methods

1H, 13C and 19F NMR analyses were conducted on a Bruker Avance 400 MHz NMR spectrometer or a JEOL ECZ400s 400 MHz NMR spectrometer using deuterated chloroform, deuterated dimethyl sulfoxide, or deuterated acetone as solvent. The shift (6) of each signal was measured in parts per million (ppm) relative the residual solvent peak, and the multiplicity reported together with the associated coupling constant (J), where applicable.

### Example 3: Waters Acquity UPLC-MS Analysis Methodology

### General Analytical Methods

1H, ¹³C and ¹⁹F NMR analyses were conducted on a Bruker Avance 400 MHz NMR spectrometer or a JEOL ECZ400s 400 MHz NMR spectrometer using deuterated acetonitrile, deuterated dimethyl sulfoxide, deuterated methanol or deuterated acetone as solvent. The shift (6) of each signal was measured in parts per million (ppm) relative the residual solvent peak, and the multiplicity reported together with the associated coupling constant (J), where applicable.

### Waters Acquity UPLC-MS Analysis Methodology

UPLC-MS analysis was carried out on a Waters Acquity UPLC system consisting of an Acquity I-Class Sample Manager-FL, Acquity I-Class Binary Solvent Manager and an Acquity UPLC Column Manager. UV detection was afforded using an Acquity UPLC PDA detector (scanning from 210 to 400 nm), whilst mass detection was achieved using an Acquity QDa detector (mass scanning from 100-1250 Da; positive and negative modes simultaneously), and ELS detection was achieved using an Acquity UPLC ELS Detector.

Samples were prepared by dissolution (with or without sonication) into 1 mL of 50% (v/v) MeCN in water. The resulting solutions were then filtered through a 0.2 m syringe filter before submitting for analysis. All the solvents, including formic acid and 36% ammonia solution, were purchased as the HPLC grade.

Specific differences between methods are as follows:

### Acidic 2 Min

0.1% v/v Formic acid in 10 mM ammonium formate [Eluent A]; 0.1% v/v Formic acid in MeCN [Eluent B]; flow rate 0.8 mL/min; column oven 50° C.; sample manager 20° C.; injection volume 2 L and 1.5 minutes equilibration time between samples, on a Waters Acquity UPLC BEH C18 column (2.1×50 mm, 1.7 μm).

### Gradient:

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Acidic 4 Min

0.1% v/v formic acid in 10 mM ammonium formate [Eluent A]; 0.10% v/v formic acid in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Basic 2 Min

0.1% ammonia in water [Eluent A]; 0.10% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.25 | 95 | 5 |
| 1.25 | 5 | 95 |
| 1.55 | 5 | 95 |
| 1.65 | 95 | 5 |
| 2.00 | 95 | 5 |

### Basic 4 Min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 columnm (2.1×50 mm, 1.7 μm).

| Time (min) | Eluent A (%) | Eluent B (%) |
|---|---|---|
| 0.00 | 95 | 5 |
| 0.25 | 95 | 5 |
| 2.75 | 5 | 95 |
| 3.25 | 5 | 95 |
| 3.35 | 95 | 5 |
| 4.00 | 95 | 5 |

### Basic 6 Min

0.1% ammonia in water [Eluent A]; 0.1% ammonia in MeCN [Eluent B]; flow rate 0.8 mL/min; column oven 50° C.; sample manager 20° C.; injection volume 2 μL and 1.5 minutes equilibration time between samples on a Waters Acquity UPLC BEH C18 column (2.1×50 mm, 1.7 μm).

| Time (min) | Eluent A (%) | Eluent B (%) |
|---|---|---|
| 0.00 | 95 | 5 |
| 0.30 | 95 | 5 |
| 6.00 | 5 | 95 |
| 6.10 | 95 | 5 |
| 7.00 | 95 | 5 |

### Chiral Prep HPLC

### Purification Method A (P.M. A)

| | |
|---|---|
| Column Details | Chiralpak IG (21 mm × 250 mm, 5 μm) |
| Column Temperature | 40° C. |
| Flow Rate | 50 mL/min |
| BPR | 100 BarG |
| Detector Wavelength | 233 nm |
| Injection Volume | 1000 μL (28 mg) |
| Isocratic Conditions | 40:60 MeOH:CO₂ (0.2% v/v NH₃) |

### Purification Method B (P.M. B)

| | |
|---|---|
| Column Details | Chiralpak IG (21 mm × 250 mm, 5 μm) |
| Column Temperature | 40° C. |
| Flow Rate | 50 mL/min |
| BPR | 125 BarG |
| Detector Wavelength | 210 nm |
| Injection Volume | 660 μL (6.4 mg) |
| Isocratic Conditions | 25:75 MeOH:CO₂ (0.2% v/v NH₃) |

### Purification Method C (P.M. B)

| | |
|---|---|
| Column Details | Chiralpak IH (21 mm × 250 mm, 5 μm) |
| Column Temperature | Ambient |
| Flow Rate | 21 mL/min |
| BPR | 100 BarG |
| Detector Wavelength | 210 nm |
| Injection Volume | 400 μL (5.6 mg) |
| Isocratic Conditions | 50:50 HEX:EtOH (0.2% v/v NH₃) |

### Example 4: Synthesis and Characterization

### Cesium 6-(1H-imidazol-1-yl)-4-methylpicolinate 4

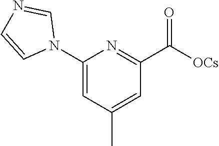

A stirred mixture of 6-bromo-4-methyl-2-pyridinecarboxylic acid (939 mg, 4.35 mmol), imidazole (355 mg, 5.22 mmol), copper (I) iodide (83 mg, 0.435 mmol) and cesium carbonate (2.83 g, 8.69 mmol) in DMSO (7.5 mL) was heated at 125° C. for 18 h. The supernatant was removed by pipette and the residue washed with MeCN (10 mL). The MeCN washings were combined with the DMSO supernatant, and the resulting precipitate was filtered and dried under vacuum to afford the desired product cesium 6-(1H-imidazol-1-yl)-4-methylpicolinate 4 (869 mg, 2.69 mmol, 60% yield) as a brown solid. ¹H NMR (400 MHz, DMSO-d₆) δ 8.47 (s, TH), 7.92 (s, TH), 7.53 (s, TH), 7.48 (s, TH), 7.09 (s, TH), 2.37 (s, 3H). UPLC-MS (Acidic 2 min): rt=0.33 min m/z (ES+) 204.0 [M+H]⁺.

### Lithium 6-(1H-imidazol-1-yl)-4-methylpicolinate 5

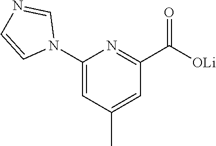

Copper (I) iodide (352 mg, 1.85 mmol) was added to a de-gassed suspension of 6-chloro-4-methylpyridine-2-carboxylic acid (3.08 g, 18.0 mmol), imidazole (2.45 g, 36.0 mmol) and cesium carbonate (11.45 g, 35.1 mmol) in DMSO (18 mL) and the mixture was heated at 140° C. for 96 h. On cooling 5M aq. HCl (3.4 mL, 17 mmol) was added the mixture was filtered, and the solids were washed with MeOH (3 mL). The combined washings were evaporated to give a brown liquid (6.00 g) containing cesium; 6-imidazol-1-yl-4-methyl-pyridine-2-carboxylate which was used directly in the next step.

A solution of crude cesium; 6-imidazol-1-yl-4-methyl-pyridine-2-carboxylate (2.10 g, 6.27 mmol) in DMSO (16.8 mL) and methanol (35 mL) was heated at 60° C. for 48 h. Chlorotrimethylsilane (3.5 mL, 27.6 mmol) was added and the mixture was stirred for 96 h. The solvent was evaporated, 1M aq. K₂CO₃ solution (20 mL) was added and the mixture was extracted with DCM (6×40 mL). The organic layers were dried (Na₂SO₄), filtered and concentrated to dryness to give crude product. The crude material was applied to an SCX cartridge (2×20 g, pre-washed with MeOH) and eluted with MeOH (2 cartridge volumes) then 2M NH₃ in MeOH (3 cartridge volumes). The basic fractions were evaporated to give a yellow gummy solid (2.01 g). The crude material was purified by column chromatography over silica eluting with a gradient of MeOH (0% to 8%; v/v) in DCM to afford the desired product methyl 6-imidazol-1-yl-4-methyl-pyridine-2-carboxylate (917 mg, 4.22 mmol, 67% yield) as an off-white solid.

A solution of lithium hydroxide (174 mg, 4.15 mmol) in water (3 mL) was added to a solution of methyl 6-imidazol-1-yl-4-methyl-pyridine-2-carboxylate (890 mg, 4.10 mmol) in THF (8 mL) and methanol (1.5 mL) and the resultant yellow solution was stirred at room temperature for 72 h. The solvent was removed under reduced pressure, the residue was triturated with MTBE (2 mL) then freeze-dried to afford the desired product lithium 6-(1H-imidazol-1-yl)-4-methylpicolinate 5 (914 mg, 4.06 mmol, 99% yield) as a yellow solid. ¹H NMR (400 MHz, DMSO-d₆) δ 8.57 (s, 1H), 8.00 (s, 1H), 7.76 (s, 1H), 7.63 (s, 1H), 7.11 (s, 1H), 2.43 (s, 3H). UPLC-MS (Acidic 2 min): rt=0.44 min, m/z (ES+) 204.1 [M+H]⁺.

### 6-(1H-Imidazol-1-yl)-4-methyl-N-(piperidin-4-yl)picolinamide hydrochloride 6

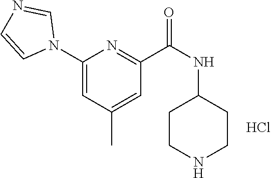 [SMILES: Cc1cc(C(=O)NC2CCNCC2)nc(-n2ccnc2)c1 ; Cl]

N,N-Diisopropylethylamine (1.03 mL, 5.92 mmol) was added to a solution of cesium 6-(1H-imidazol-1-yl)-4-methylpicolinate (600 mg, 1.79 mmol), 4-amino-1-Boc-piperidine (828 mg, 4.13 mmol) and O-(7-Azabenzotriazol-1-yl)-N,N,N′,N′-tetramethyluronium hexafluorophosphate (1.68 g, 4.43 mmol) in DMF (3.6 mL) and stirred at 21° C. for 2 h. The crude solution was filtered through an SCX cartridge (2 g, pre-washed with MeOH) and washed with MeOH (2 cartridge volumes) then eluted with 2M NH₃ in MeOH (4 cartridge volumes). The product fraction was concentrated under reduced pressure, then taken up in 4M HCl in 1,4-dioxane (1.5 mL) and stirred at room temperature for 1 h. The precipitate was filtered to afford the desired product 6-(1H-imidazol-1-yl)-4-methyl-N-(piperidin-4-yl)picolinamide hydrochloride 6 which was used without further purification. UPLC-MS (Basic 2 min): rt=0.78 min, m/z=m/z (ES+) 286.2 [M+H]⁺.

### Potassium 6-(1H-imidazol-1-yl)-4-methoxylpicolinate 7

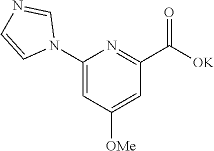

A mixture of methyl 6-chloro-4-methyoxypicolinate (252 mg, 1.25 mmol), imidazole (189 mg, 2.78 mmol), copper (I) iodide (27 mg, 0.436 mmol) and potassium carbonate (371 mg, 2.68 mmol) in DMSO (3 mL) was heated at 140° C. for 60 h. The mixture was cooled, diluted with methanol (10 ml), filtered and the residue was washed with methanol (20 mL). The combined filtrate and washings were concentrated under reduced pressure. Acetonitrile (10 mL) was added, and the precipitate formed filtered and dried under vacuum to afford the desired product potassium 6-(1H-imidazol-1-yl)-4-methoxylpicolinate 7 (307 mg, 0.93 mmol, 75% yield) as a brown solid. ¹H NMR (400 MHz, MeOD-d₄) δ 8.12 (broad s, 1H), 8.93 (s, 1H), 7.57 (d, J=1.9 Hz, 1H), 7.26 (d, J=2.1 Hz, 1H), 7.62-7.13 (2H, m), 3.98 (s, 3H). UPLC-MS (Basic 2 min): rt=0.45 min, m/z (ES+) 220.1 [M+H]⁺

### Lithium 2-(1H-imidazol-1-yl)-6-methylpyrimidine-4-carboxylate 8

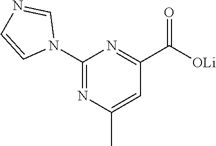

Methyl 2-chloro-6-methylpyrimidine-4-carboxylate (1.50 g, 8.04 mmol), imidazole (1.09 g, 16.1 mmol) and N,N-diisopropylethylamine (4.2 mL, 24.1 mmol) were dissolved in NMP (30 mL) and heated at 100° C. for 18 h. This was diluted with water (30 ml) and extracted with ethyl acetate (2×30 ml). The combined organic layers were washed with water (30 ml×2), dried (Na₂SO₄), filtered and concentrated under reduced pressure to give the desired ester intermediate (1.4 g) as a yellow solid. The mixture was dissolved in THF (7.5 mL) and water (15 mL) and lithium hydroxide (0.34 g, 8.04 mmol) was added and stirred at rt for 5 h. This was concentrated under reduced pressure to afford the desired product lithium 2-(1H-imidazol-1-yl)-6-methylpyrimidine-4-carboxylate 8 (1.20 g, 5.71 mmol, 71% yield) as a white solid. ¹H NMR (400 MHz, DMSO-d₆) δ 8.52 (t, J=1.1 Hz, 1H), 7.91 (t, J=1.3 Hz, 1H), 7.43-7.39 (m, 1H), 7.11 (dd, J=1.5, 1.0 Hz, 1H). 3H unobserved.

### 2-(1H-Imidazol-1-yl)pyrimidine-4-carboxylic acid 9

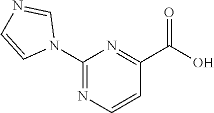 [SMILES: O=C(O)c1ccnc(-n2ccnc2)n1]

Methyl 2-chloropyrimidine-4-carboxylate (500 mg, 2.90 mmol), N,N-diisopropylethylamine (1.6 mL, 9.19 mmol) and imidazole (237 mg, 3.48 mmol) were combined in NMP (10 mL) and the resultant solution was heated to 100° C. for 2 h. Further imidazole (160 mg, 2.35 mmol) was added and heating continued for 21 h. The reaction mixture was cooled, then excess water was added and the mixture was concentrated under reduced pressure which caused the formation of a precipitate. This was collected by filtration, washed with MeCN and dried under vacuum to afford the desired product 2-(1H-imidazol-1-yl)pyrimidine-4-carboxylic acid 9 (504 mg, 2.70 mmol, 91% yield) as a brown solid. ¹H NMR (400 MHz, DMSO-d₆) δ 9.09 (d, J=5.0 Hz, 1H), 8.63 (t, J=1.1 Hz, 1H), 7.99 (t, J=1.4 Hz, 1H), 7.91 (d, J=4.9 Hz, 1H), 7.20-7.15 (m, 1H). 1H unobserved.

### 1H-Imidazole 2-(1H-imidazol-1-yl)-6-(trifluoromethyl)pyrimidine-4-carboxylate 10

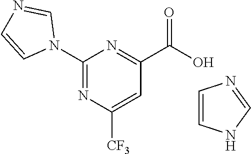

To a solution of 2-chloro-6-(trifluoromethyl)pyrimidine-4-carboxylic acid (200 mg, 0.883 mmol) in MeCN (8 mL) was added N,N-diisopropylethylamine (0.46 mL, 2.65 mmol) followed by imidazole (240 mg, 3.53 mmol) and the resultant solution was heated at 60° C. for 18 h. The crude material was directly purified by column chromatography over C18 eluting with a gradient of MeCN (0.1% NH₃) (5% to 15%; v/v) in water (0.1% NH₃) to afford the desired product 1H-imidazole 2-(1H-imidazol-1-yl)-6-(trifluoromethyl)pyrimidine-4-carboxylate 10 (195 mg, 0.568 mmol, 64% yield) as an off-white solid. ¹H NMR (400 MHz, DMSO-d₆) δ 8.57 (s, 1H), 8.31 (s, 1H), 8.03 (s, 1H), 7.94 (s, 1H), 7.33 (s, 2H), 7.17 (s, 1H). NH/OH not observed. UPLC-MS (Basic 4 min): rt=0.80 min, m/z (ES+) 259.1 [M+H]⁺

### General Method A

N,N-Diisopropylethylamine (2-3 eq.) was added to a solution of corresponding carboxylate salt or carboxylic acid (1.0 eq.), amine or amine hydrochloride (1-1.4 eq.) and 0-(7-azabenzotriazol-1-yl)-N,N,N,N′-tetramethyluronium hexafluorophosphate (1.5 eq.) in DMF (1.2 mL/mmol substrate) and stirred at 21° C. for 2-18 h. The crude solution was applied to an SCX cartridge (2 g, pre-washed with MeOH) and washed with MeOH (2 cartridge volumes) then eluted with 2M NH₃ in MeOH (4 cartridge volumes). This ammonia fraction was concentrated under reduced pressure to give crude product. Alternatively, the reaction mixture was concentrated under reduced pressure to give a solid residue. The crude compound was purified by column chromatography over silica eluting with a gradient of MeOH (0% to 10%; v/v) in DCM, or by reverse phase column chromatography over C18 eluting with a gradient of MeCN (0.1% NH₃) (5% to 100%; v/v) in water (0.1% NH₃), with further purification by prep HPLC or trituration from MTBE if required, to afford the desired product which was freeze dried to yield a solid.

### General Method B

1-Methylimidazole (3.5-5.5 eq.) was added to a solution of corresponding carboxylate salt or carboxylic acid (1.0 eq.), amine or amine hydrochloride (1.4 eq) and chloro-N,N,N′,N′-tetramethylformamidinium hexafluorophosphate (2.2 eq) in MeCN (2 ml) and stirred at 21° C. until reaction reached completion (0.5-18 h). The crude mixture was either purified directly by reverse phase chromatography over C18 eluting with a gradient of MeCN (0.1% NH₃) (5% to 95%; v/v) in water (0.1% NH₃), or the reaction mixture was diluted with water (10 ml) to generate the desired product after washing with further water and vacuum drying. Alternatively, if precipitation did not occur this was extracted with ethyl acetate (3×10 ml). The combined organic layers were dried (Na₂SO₄), filtered and concentrated under reduced pressure to give crude product. This was purified by reverse phase column chromatography over C18 eluting with a gradient of MeCN (0.1% NH₃) (5% to 95%; v/v) in water (0.1% NH₃), with further purification by prep HPLC if required, to afford the desired product which was freeze dried to yield a solid.

### General Method C

2-(1H-Imidazol-1-yl)-6-methyl-N-(piperidin-4-yl)isonicotinamide or 6-imidazol-1-yl-4-methyl-N-(4-piperidyl)pyridine-2-carboxamide hydrochloride 6 (1 eq.) and triethylamine (2 or 3 eq.) were dissolved in DMF (5 mL/mmol) and stirred at 0° C. for 10 mins. Sulfonyl chloride (1.2 eq.) was added portionwise and the mixture allowed to warm to room temperature and stirred for 1 h. The crude solution was filtered through an SCX cartridge (2 g, pre-washed with MeOH) and washed with MeOH (2 cartridge volumes) then eluted with 2M NH₃ in MeOH (4 cartridge volumes). The ammonia fraction was concentrated under reduced pressure, and the crude product purified by column chromatography over silica eluting with a gradient of MeOH (0% to 10%; v/v) in DCM to afford the desired product which was freeze dried to yield a solid.

### General Method D

6-Imidazol-1-yl-4-methyl-N-(4-piperidyl)pyridine-2-carboxamide hydrochloride 6 (1 eq.), carboxylic acid (1.4 eq.) and N,N-diisopropylethylamine (2.0 eq.) were dissolved in DMF (4 mL/mmol) and stirred at room temperature for 10 mins. O-(7-Azabenzotriazol-1-yl)-N,N,N′,N′-tetramethyluronium hexafluorophosphate (1.5 eq.) was added to the reaction mixture and stirred for 18 h. The crude solution was filtered through an SCX cartridge (2 g, pre-washed with MeOH) and washed with MeOH (2 cartridge volumes) then eluted with 2M NH₃ in MeOH (4 cartridge volumes). The ammonia fraction was concentrated under reduced pressure, and the crude product purified by column chromatography over silica eluting with a gradient of MeOH (0% to 10%; v/v) in DCM to afford the desired product which was freeze dried to yield a solid.

### General Method E

6-Imidazol-1-yl-4-methyl-N-(4-piperidyl)pyridine-2-carboxamide hydrochloride 6 (1 eq.), triethylamine (3 eq.) and formyl or carbamoyl chloride (1.2 eq.) in DCM (5 mL/mmol) were stirred at room temperature for 2 h. The crude solution was filtered through an SCX cartridge (2 g, pre-washed with MeOH) and washed with MeOH (2 cartridge volumes) then eluted with 2M NH₃ in MeOH (4 cartridge volumes). The ammonia fraction was concentrated under reduced pressure, and the crude product purified by column chromatography over silica eluting with a gradient of MeOH (0% to 10%; v/v) in DCM. The product fraction was concentrated under reduced pressure, and the residue dissolved in DCM, washed with sat. aq. NaHCO₃, and water. The organic layer was dried (Na₂SO₄), filtered, and concentrated to afford the desired product which was freeze dried to yield a solid.

### General Method F

Methanesulfonyl chloride (1.2 eq.) was added to a suspension of amine hydrochloride (1.0 eq.) and triethylamine (1.5-5.5 eq.) in DCM (0.5-1.5 mL) and the resultant solution was stirred for 2 h. Sat. Aq NaHCO₃ or water (2 mL) was added, and the mixture was separated. The aqueous residues were further extracted with DCM (2×2 mL) and the combined organic layers were dried (Na₂SO₄), filtered and concentrated under reduced pressure to afford crude product. The crude material was purified by either column chromatography over silica eluting with a gradient of MeOH (1% to 8%; v/v) in DCM or by reverse phase column chromatography over C₁₈ eluting with a gradient of MeCN (0.1% NH₃) (5% to 15%; v/v) in water (0.1% NH₃) to afford the desired product which was freeze dried to yield a solid.

### General Method G

4M Hydrogen chloride in dioxane (0.5-1.0 mL) was added either directly to the corresponding Boc protected amine (1.0 eq.) as a solid or as a solution in DCM (0.1 mL) and the resulting solution was stirred for 1-3 h. The solvent was then removed under reduced pressure to afford the desired product as a solid which was used directly in further steps, or where a gum was formed trituration and filtration from TBME formed the desired solid.

### General Method H

Copper (I) iodide (10-30 mol %) was added to a suspension of corresponding bromide/chloride (1.0 eq.), cesium carbonate (2.0 eq.) and imidazole (1.5-2.0 eq.) in anhydrous DMSO (1-5 mL) under N₂ and heated at 120-125° C. for 3-18 h. The crude solution was filtered through an SCX cartridge (2 g, pre-washed with MeOH) and washed with MeOH (2 cartridge volumes) then eluted with 2M NH₃ in MeOH (4 cartridge volumes). The ammonia fractions were concentrated under reduced pressure. Alternatively smaller scale reactions were concentrated directly to yield crude product. The crude material was purified by reverse phase column chromatography over C18 eluting with a gradient of MeCN (0.1% NH₃) (5% to 75%; v/v) in water (0.1% NH₃) to afford the desired product which was freeze dried to yield a solid.

### 6-(1H-Imidazol-1-yl)-4-methyl-N-(1-(methylsulfonyl)piperidin-4-yl)picolinamide (1A)

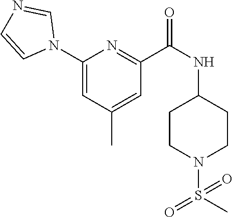 [SMILES: Cc1cc(C(=O)NC2CCN(S(C)(=O)=O)CC2)nc(-n2ccnc2)c1]

Prepared from cesium 6-(1H-imidazol-1-yl)-4-methylpicolinate 4 and 1-(methylsulfonyl)piperidin-4-amine according to General Method A to afford the desired product 6-(1H-imidazol-1-yl)-4-methyl-N-(1-(methylsulfonyl)piperidin-4-yl)picolinamide (11 mg, 0.0300 mmol, 33% yield) as a colourless solid. ¹H NMR (400 MHz, DMSO-d₆) δ 8.92 (dt, J=2.4, 1.1 Hz, 1H), 8.75-8.58 (m, 1H), 8.23 (dt, J=3.0, 1.5 Hz, 1H), 7.95-7.76 (m, 2H), 7.15 (dd, J=2.6, 1.3 Hz, 1H), 4.08-3.89 (m, 1H), 3.62 (d, J=11.8 Hz, 2H), 2.91-2.88 (m, 3H), 2.87-2.81 (m, 2H), 2.48 (d, J=3.2 Hz, 3H), 1.91-1.74 (m, 4H). UPLC-MS (Basic 4 min): rt=1.27 min, m/z (ES+) 364.2 [M+H]⁺.

### N-(1-(Ethylsulfonyl)piperidin-4-yl)-6-(1H-imidazol-1-yl)-4-methylpicolinamide (2A)

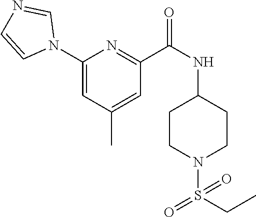 [SMILES: CCS(=O)(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and ethanesulfonyl chloride according to General Method C to afford the desired product N-(1-(ethylsulfonyl)piperidin-4-yl)-6-(1H-imidazol-1-yl)-4-methylpicolinamide (11 mg, 0.0290 mmol, 27% yield) as a colourless solid. ¹H NMR (400 MHz, DMSO-d₆) δ 8.92 (d, J=1.1 Hz, 1H), 8.67 (d, J=8.7 Hz, 1H), 8.23 (d, J=1.4 Hz, 1H), 7.89-7.87 (m, 1H), 7.82 (dd, J=1.3, 0.7 Hz, 1H), 7.15 (dd, J=1.5, 0.9 Hz, 1H), 4.12-3.93 (m, 1H), 3.67 (d, J=12.5 Hz, 2H), 3.07 (q, J=7.4 Hz, 2H), 2.95 (td, J=12.2, 2.8 Hz, 2H), 2.48 (d, J=0.7 Hz, 3H), 1.90-1.71 (m, 4H), 1.23 (t, J=7.4 Hz, 3H). UPLC-MS (Basic 4 min): rt=1.26 min, m/z (ES+) 378.3 [M+H]⁺

### N-(1-(cyclopropylsulfonyl)piperidin-4-yl)-6-(1H-imidazol-1-yl)-4-methylpicolinamide (3A)

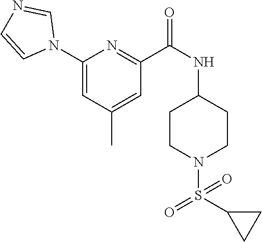 [SMILES: Cc1cc(C(=O)NC2CCN(S(=O)(=O)C3CC3)CC2)nc(-n2ccnc2)c1]

Prepared from 6-(1H-imidazol-1-yl)-4-methyl-N-(piperidin-4-yl)picolinamide hydrochloride 6 and cyclopropanesulfonyl chloride according to General Method C to afford the desired product N-(1-(cyclopropylsulfonyl)piperidin-4-yl)-6-(1H-imidazol-1-yl)-4-methylpicolinamide (7.8 mg, 0.0200 mmol, 18% yield) as a colourless solid. ¹H NMR (400 MHz, DMSO-d₆) δ 8.92 (dd, J=1.2, 1.2 Hz, 1H), 8.67 (d, J=8.6 Hz, 1H), 8.22 (dd, J=1.4, 1.4 Hz, 1H), 7.89-7.87 (m, 1H), 7.83-7.81 (m, 1H), 7.18-7.12 (dd, J=1.4, 1.0 Hz, 1H), 4.06-3.95 (m, 1H), 3.72-3.61 (m, 2H), 3.01-2.92 (m, 2H), 2.65-2.55 (m, 1H), 2.48 (s, 3H), 1.93-1.70 (m, 4H), 1.05-0.90 (m, 4H). UPLC-MS (Basic 4 min): rt=1.31 min, m/z (ES+) 390.3 [M+H]⁺

### 6-(1H-Imidazol-1-yl)-N-(1-((2-methoxyethyl)sulfonyl)piperidin-4-yl)-4-methylpicolinamide (4A)

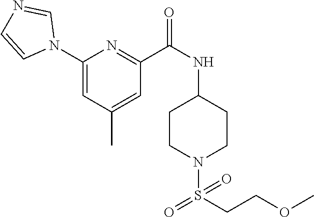 [SMILES: COCCS(=O)(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and 2-methoxy-1-ethanesulfonyl chloride according to General Method C to afford the desired product 6-(1H-imidazol-1-yl)-N-(1-((2-methoxyethyl)sulfonyl)piperidin-4-yl)-4-methylpicolinamide (3.2 mg, 0.00758 mmol, 7% yield) as a colourless solid. ¹H NMR (400 MHz, ACETONE-d₆) δ 8.61 (dd, J=1.1, 1.1 Hz, 1H), 8.41 (d, J=8.5 Hz, 1H), 7.99 (dd, J=1.2, 1.2 Hz, 1H), 7.91 (dq, J=1.3, 0.6 Hz, 1H), 7.79 (dq, J=1.5, 0.6 Hz, 1H), 7.11 (dd, J=1.2, 0.9 Hz, 1H), 4.14-4.03 (m, 1H), 3.80-3.72 (m, 2H), 3.73 (t, J=6.1 Hz, 2H), 3.35 (s, 3H), 3.28 (t, J=6.1 Hz, 2H), 3.08-2.99 (m, 2H), 2.54 (t, J=0.7 Hz, 3H), 2.10-1.97 (m, 2H), 1.85-1.73 (m, 2H). UPLC-MS (Basic 2 min): rt=0.88 min, m/z (ES+) 408.3 [M+H]⁺

### N-(1-(Cyclohexylsulfonyl)piperidin-4-yl)-6-(1H-imidazol-1-yl)-4-methylpicolinamide (5A)

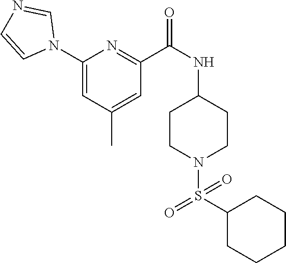 [SMILES: Cc1cc(C(=O)NC2CCN(S(=O)(=O)C3CCCCC3)CC2)nc(-n2ccnc2)c1]

Prepared from 6-(1H-imidazol-1-yl)-4-methyl-N-(piperidin-4-yl)picolinamide hydrochloride 6 and cyclohexanesulfonyl chloride according to General Method C to afford the desired product N-(1-(cyclohexylsulfonyl)piperidin-4-yl)-6-(1H-imidazol-1-yl)-4-methylpicolinamide (3.6 mg, 0.00792 mmol, 7% yield) as a colourless solid. ¹H NMR (400 MHz, ACETONE-d₆) δ 8.61 (dd, J=1.2, 1.2 Hz, 1H), 8.42 (d, J=8.6 Hz, 1H), 7.99 (dd, J=1.3, 1.3 Hz, 1H), 7.90 (dq, J=1.3, 0.8 Hz, 1H), 7.79 (dq, J=1.0, 0.8 Hz, 1H), 7.11 (dd, J=1.2, 0.9 Hz, 1H), 4.12 (tdt, J=11.3, 8.5, 4.2 Hz, 1H), 3.86-3.76 (m, 2H), 3.16-2.97 (m, 3H), 2.54 (t, J=0.7 Hz, 3H), 2.14-2.06 (m, 2H), 2.01-1.93 (m, 2H), 1.89-1.80 (m, 2H), 1.80-1.63 (m, 3H), 1.53-1.14 (m, 5H). UPLC-MS (Basic 2 min): rt=1.04 min, m/z (ES+) 432.4 [M+H]⁺

### 6-(1H-Imidazol-1-yl)-4-methyl-N-(1-(propylsulfonyl)piperidin-4-yl)picolinamide (6A)

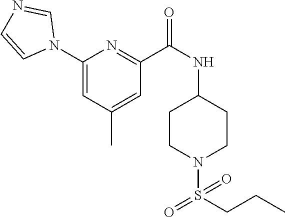 [SMILES: CCCS(=O)(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and 1-propylsulfonyl chloride according to General Method C to afford the desired product 6-(1H-imidazol-1-yl)-4-methyl-N-(1-(propylsulfonyl)piperidin-4-yl)picolinamide (10 mg, 0.0259 mmol, 24% yield) as a colourless solid. ¹H NMR (400 MHz, DMSO-d₆) δ 8.92 (dd, J=1.2, 1.2 Hz, 1H), 8.67 (d, J=8.7 Hz, 1H), 8.23 (dd, J=1.4, 1.4 Hz, 1H), 7.88 (dd, J=1.1, 1.1 Hz, 1H), 7.84-7.76 (m, 1H), 7.15 (dd, J=1.2, 1.2 Hz, 1H), 4.06-3.95 (m, 1H), 3.73-3.60 (m, 2H), 3.08-2.99 (m, 2H), 2.97-2.88 (m, 2H), 2.48 (dd, J=0.8, 0.8 Hz, 3H), 1.89-1.64 (m, 6H), 1.00 (t, J=7.4 Hz, 3H). UPLC-MS (Basic 4 min): rt=1.40 min, m/z (ES+) 392.2 [M+H]⁺

### 6-(1H-Imidazol-1-yl)-4-methyl-N-(1-(methylsulfonyl)azetidin-3-yl)picolinamide (7A)

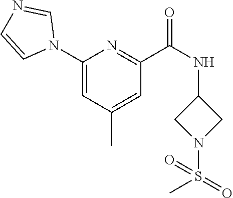 [SMILES: Cc1cc(C(=O)NC2CN(S(C)(=O)=O)C2)nc(-n2ccnc2)c1]

Prepared from cesium 6-(1H-imidazol-1-yl)-4-methylpicolinate 4 and (1-methylsulfonylazetidin-3-yl)ammonium chloride according to General Method A to afford the desired product 6-(1H-imidazol-1-yl)-4-methyl-N-(1-(methylsulfonyl)azetidin-3-yl)picolinamide (27 mg, 0.0794 mmol, 60% yield) as a colourless solid. ¹H NMR (400 MHz, DMSO-d₆) δ 9.30 (d, J=7.1 Hz, 1H), 8.93 (s, 1H), 8.25 (s, 1H), 7.92 (s, 1H), 7.81 (s, 1H), 7.16 (s, 1H), 4.83-4.69 (m, 1H), 4.21-4.06 (m, 4H), 3.09 (s, 3H), 2.48 (s, 3H). UPLC-MS (Acidic 4 min): rt=0.89 min, m/z (ES+) 336.2 [M+H]⁺

### N-(4,4-Difluorocyclohexyl)-6-(1H-imidazol-1-yl)-4-methylpicolinamide (8A)

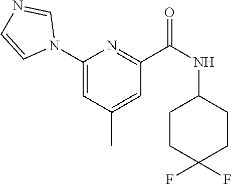 [SMILES: Cc1cc(C(=O)NC2CCC(F)(F)CC2)nc(-n2ccnc2)c1]

Prepared from cesium 6-(H-imidazol-1-yl)-4-methylpicolinate 4 and 4,4-difluorocyclohexanamine according to General Method A to afford the desired product N-(4,4-difluorocyclohexyl)-6-(1H-imidazol-1-yl)-4-methylpicolinamide (4.0 mg, 0.0124 mmol, 17% yield) as a colourless solid. ¹H NMR (400 MHz, DMSO-d₆) δ 8.93 (s, 1H), 8.64 (d, J=8.8 Hz, 1H), 8.23 (s, 1H), 7.88 (s, 1H), 7.82 (s, 1H), 7.14 (s, 1H), 4.15-3.96 (m, 1H), 2.14-1.77 (m, 8H). UPLC-MS (Basic 4 min): rt=1.53 min, m/z (ES+) 321.2 [M+H]⁺

### 6-(1H-Imidazol-1-yl)-4-methyl-N-(tetrahydro-2H-pyran-3-yl)picolinamide (9A)

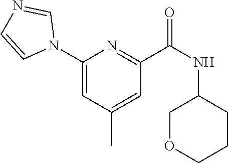 [SMILES: Cc1cc(C(=O)NC2CCCOC2)nc(-n2ccnc2)c1]

Prepared from cesium 6-(1H-imidazol-1-yl)-4-methylpicolinate 4 and tetrahydropyran-3-yl amine hydrochloride according to General Method A to afford the desired product 6-(1H-imidazol-1-yl)-4-methyl-N-(tetrahydro-2H-pyran-3-yl)picolinamide (3.5 mg, 0.0121 mmol, 16% yield) as a colourless solid. ¹H NMR (400 MHz, DMSO-d₆) δ 8.89 (q, J=1.1 Hz, 1H), 8.52 (d, J=8.7 Hz, 1H), 8.20 (s, 1H), 7.89 (s, 1H), 7.82 (s, 1H), 7.15 (s, 1H), 4.06-3.90 (m, 1H), 3.84-3.67 (m, 2H), 1.95-1.52 (m, 4H), 1.29-1.16 (m, 1H). 4 protons unobserved. UPLC-MS (Basic 4 min): rt=0.90 min, m/z (ES+) 287.2 [M+H]⁺

### N-(1-Acetylpiperidin-4-yl)-6-(1H-imidazol-1-yl)-4-methylpicolinamide (10A)

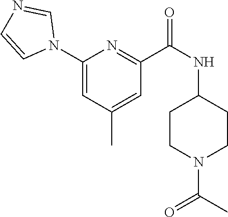 [SMILES: CC(=O)N1CCC(NC(=O)c2cc(C)cc(-n3ccnc3)n2)CC1]

Prepared from cesium 6-(1H-imidazol-1-yl)-4-methylpicolinate 4 and 1-(4-aminopiperidin-1-yl)ethan-1-one according to General Method A to afford the desired product N-(1-acetylpiperidin-4-yl)-6-(1H-imidazol-1-yl)-4-methylpicolinamide (17 mg, 0.0514 mmol, 57% yield) as a colourless solid. ¹H NMR (400 MHz, DMSO-d₆) δ 8.91 (s, 1H), 8.62 (d, J=9.1 Hz, 1H), 8.22 (dt, J=4.0, 1.4 Hz, 1H), 7.91-7.80 (m, 2H), 7.18-7.11 (m, 1H), 4.42 (d, J=13.3 Hz, 1H), 4.20-4.04 (m, 1H), 3.87 (d, J=13.8 Hz, 1H), 3.14 (t, J=13.1 Hz, 1H), 2.63 (t, J=13.1 Hz, 1H), 2.48 (d, J=3.6 Hz, 3H), 2.06-2.00 (m, 3H), 1.88-1.44 (m, 4H). UPLC-MS (Basic 4 min): rt=1.17 min, m/z (ES+) 328.1 [M+H]⁺

### 6-(1H-Imidazol-1-yl)-4-methyl-N-(piperidin-4-yl)picolinamide (11A)

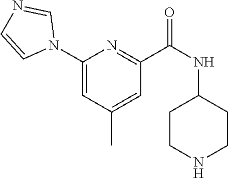 [SMILES: Cc1cc(C(=O)NC2CCNCC2)nc(-n2ccnc2)c1]

A small amount of 6-(1H-imidazol-1-yl)-4-methyl-N-(piperidin-4-yl)picolinamide hydrochloride 6 was purified by column chromatography over C18 eluting with a gradient of MeCN (0.1% NH₃) (5% to 100%; v/v) in water (0.1% NH₃) to afford the desired product 6-(1H-imidazol-1-yl)-4-methyl-N-(piperidin-4-yl)picolinamide as a colourless solid. ¹H NMR (400 MHz, DMSO-d₆) δ 8.94-8.90 (m, 1H), 8.60 (d, J=8.6 Hz, 1H), 8.23 (dd, J=1.3, 1.3 Hz, 1H), 7.91-7.77 (m, 2H), 7.14 (dd, J=1.2, 1.2 Hz, 1H), 3.97-3.79 (m, 1H), 3.17 (s, 1H), 3.07-2.94 (m, 2H), 2.62-2.52 (m, 2H), 2.47 (s, 3H), 1.77-1.54 (m, 4H). UPLC-MS (Basic 2 min): rt=0.73 min, m/z (ES+) 286.2 [M+H]⁺

### N-Cyclobutyl-6-(1H-imidazol-1-yl)-4-methylpicolinamide (12A)

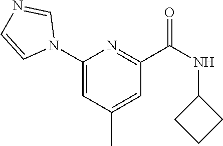 [SMILES: Cc1cc(C(=O)NC2CCC2)nc(-n2ccnc2)c1]

Prepared from cesium 6-(1H-imidazol-1-yl)-4-methylpicolinate 4 and cyclobutylamine according to General Method A to afford the desired product N-cyclobutyl-6-(1H-imidazol-1-yl)-4-methylpicolinamide (9.1 mg, 0.0349 mmol, 14% yield) as a colourless solid. ¹H NMR (400 MHz, DMSO-d₆) δ 8.94 (dd, J=1.2, 1.0 Hz, 1H), 8.93 (d, J=8.4 Hz, 1H), 8.25 (dd, J=1.4, 1.4 Hz, 1H), 7.89-7.86 (m, 1H), 7.80-7.78 (m, 1H), 7.15 (dd, J=1.5, 0.9 Hz, 1H), 4.56-4.32 (m, 1H), 2.47 (s, 3H), 2.29-2.15 (m, 4H), 1.76-1.61 (m, 2H). UPLC-MS (Basic 2 min): rt=0.93 min, m/z (ES+) 257.2 [M+H]⁺

### N-(1-Butyrylpiperidine-4-yl)-6-(1H-imidazol-1-yl)-4-methylpicolinamide (13A)

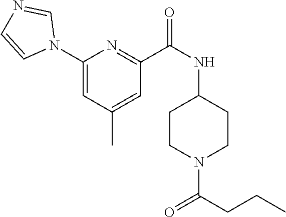 [SMILES: CCCC(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and butyric acid according to General Method D to afford the desired product N-(1-butyrylpiperidin-4-yl)-6-(1H-imidazol-1-yl)-4-methylpicolinamide (14 mg, 0.0382 mmol, 31% yield) as a colourless solid. ¹H NMR (400 MHz, DMSO-d₆) δ 8.91 (t, J=1.1 Hz, 1H), 8.61 (d, J=8.8 Hz, 1H), 8.22 (t, J=1.4 Hz, 1H), 7.94-7.78 (m, 2H), 7.14 (dd, J=1.5, 0.9 Hz, 1H), 4.45 (d, J=13.1 Hz, 1H), 4.17-4.06 (m, 1H), 3.92 (d, J=13.8 Hz, 1H), 3.11 (t, J=12.5 Hz, 1H), 2.70-2.56 (m, 1H), 2.48 (t, J=0.7 Hz, 3H), 2.31 (td, J=7.3, 2.1 Hz, 2H), 1.81 (d, J=17.0 Hz, 2H), 1.70-1.46 (m, 4H), 0.91 (t, J=7.4 Hz, 3H). UPLC-MS (Basic 4 min): rt=1.29 min, m/z (ES+) 356.3 [M+H]⁺

### 6-(1H-Imidazol-1-yl)-4-methyl-N-(1-(N-methylsulfamoyl)piperidin-4-yl)picolinamide (14A)

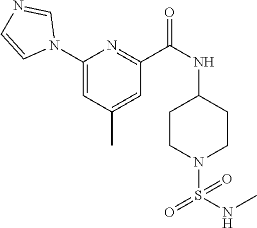 [SMILES: CNS(=O)(=O)N1CCC(NC(=O)c2cc(C)cc(-n3ccnc3)n2)CC1]

To a solution of 6-imidazol-1-yl-4-methyl-N-(4-piperidyl)pyridine-2-carboxamide hydrochloride 6 (24 mg, 0.0746 mmol) in DCM (1 mL) were added methylsulfamoyl chloride (58 mg, 0.447 mmol), triethylamine (0.062 mL, 0.447 mmol) and 4-dimethylaminopyridine (1.8 mg, 0.0149 mmol) at room temperature. The reaction mixture was allowed to stir at 50° C. for 18 h. The reaction was then heated at 60° C. for 48 h. The reaction mixture was applied to an SCX cartridge (2 g, pre-washed with MeOH), washed with MeOH (2 cartridge volumes) then eluted with 2M NH₃ in MeOH (4 cartridge volumes). The ammonia fraction was concentrated under reduced pressure to give crude product. This was purified by prep-HPLC to afford the desired product 6-(1H-imidazol-1-yl)-4-methyl-N-(1-(N-methylsulfamoyl)piperidin-4-yl)picolinamide (1.0 mg, 0.00263 mmol, 3% yield) as a colourless solid. ¹H NMR (400 MHz, ACETONE-d₆) δ 8.60 (s, 1H), 8.40 (s, 1H), 7.98 (s, 1H), 7.91 (dd, J=1.3, 0.7 Hz, 1H), 7.79 (dd, J=1.3, 0.8 Hz, 1H), 7.11 (s, 1H), 5.99 (s, 1H), 4.10-4.01 (m, 1H), 3.75-3.66 (m, 2H), 3.01-2.88 (m, 2H), 2.70 (d, J=5.1 Hz, 3H), 2.54 (t, J=0.7 Hz, 3H), 2.02-1.97 (m, 2H), 1.84-1.74 (m, 2H). UPLC-MS (Basic 4 min): rt=1.19 min, m/z (ES+) 379.3 [M+H]⁺

### 6-(1H-Imidazol-1-yl)-4-methoxy-N-(1-(methylsulfonyl)piperidin-4-yl)picolinamide (15A)

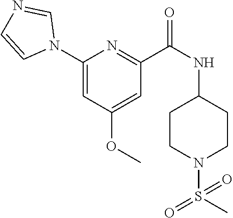 [SMILES: COc1cc(C(=O)NC2CCN(S(C)(=O)=O)CC2)nc(-n2ccnc2)c1]

Prepared from potassium 6-(1H-imidazol-1-yl)-4-methoxylpicolinate 7 and 1-(methylsulfonyl)-4-piperidinamine according to General Method A to afford the desired product 6-(1H-imidazol-1-yl)-4-methoxy-N-(1-(methylsulfonyl)piperidin-4-yl)picolinamide (21 mg, 0.0513 mmol, 9% yield) as a beige solid. ¹H NMR (400 MHz, DMSO-d₆) δ 8.95 (d, J=1.2 Hz, 1H), 8.67 (d, J=8.6 Hz, 1H), 8.27 (q, J=1.3 Hz, 1H), 7.55 (dd, J=2.2, 1.0 Hz, 1H), 7.48 (dd, J=2.1, 1.0 Hz, 1H), 7.14 (d, J=1.3 Hz, 1H), 4.01-3.97 (m, 4H), 3.62 (d, J=11.8 Hz, 2H), 2.89 (d, J=1.0 Hz, 3H), 2.88-2.82 (m, 2H), 1.92-1.76 (m, 4H). UPLC-MS (Acidic 4 min): rt=0.95 min, m/z (ES+) 380.3 [M+H]⁺

### 6-(1H-Imidazol-1-yl)-N-(1-(methylsulfonyl)piperidin-4-yl)picolinamide (16A)

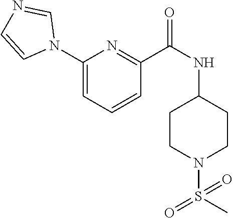 [SMILES: CS(=O)(=O)N1CCC(NC(=O)c2cccc(-n3ccnc3)n2)CC1]

Prepared from 6-imidazol-1-yl-pyridine-2-carboxylic acid and 1-(methylsulfonyl)-4-piperidinamine according to General Method A to yield 6-(1H-imidazol-1-yl)-N-(1-(methylsulfonyl)piperidin-4-yl)picolinamide (20 mg, 0.0572 mmol, 22% yield) as a colourless solid. ¹H NMR (400 MHz, DMSO-d₆) δ 8.99-8.93 (m, 1H), 8.70 (d, J=8.6 Hz, 1H), 8.28-8.24 (m, 1H), 8.17 (td, J=8.0, 1.4 Hz, 1H), 8.04-7.94 (m, 2H), 7.16 (q, J=1.3 Hz, 1H), 4.08-3.92 (m, 1H), 3.62 (d, J=12.0 Hz, 2H), 2.91-2.81 (m, 5H), 1.94-1.74 (m, 4H). UPLC-MS (Basic 4 min): rt=1.06 min, m/z (ES+) 350.1 [M+H]⁺

### Compound 17A

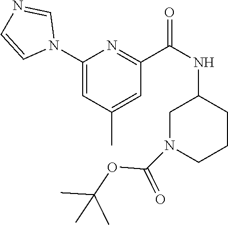 [SMILES: Cc1cc(C(=O)NC2CCCN(C(=O)OC(C)(C)C)C2)nc(-n2ccnc2)c1]

### N-(1-(N,N-Dimethylsulfamoyl)piperidin-4-yl)-6-(1H-imidazol-1-yl)-4-methylpicolinamide (18A)

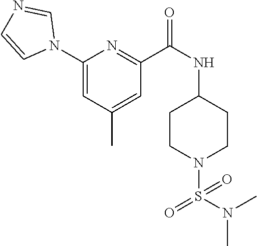 [SMILES: Cc1cc(C(=O)NC2CCN(S(=O)(=O)N(C)C)CC2)nc(-n2ccnc2)c1]

To a solution of 6-imidazol-1-yl-4-methyl-N-(4-piperidyl)pyridine-2-carboxamide hydrochloride 6 (25 mg, 0.0777 mmol) in DMF (0.42 mL) were added N,N-dimethylsulfamoyl chloride (13 mg, 0.0932 mmol) and triethylamine (0.032 mL, 0.233 mmol) at room temperature. The reaction mixture was allowed to stir at room temperature for 4 h. The reaction was diluted with sat. aq. NaHCO₃ (3 mL) and extracted with DCM (3×5 mL). The combined organic layers were washed sequentially with water (10 mL) and saturated brine solution (10 mL). The organic fraction was dried (Na₂SO₄), filtered and concentrated under reduced pressure to give crude product. This was purified by column chromatography over silica eluting with a gradient of EtOH (0% to 20%; v/v) in EtOAc to afford the desired product N-(1-(N,N-dimethylsulfamoyl)piperidin-4-yl)-6-(1H-imidazol-1-yl)-4-methylpicolinamide (7.1 mg, 0.0181 mmol, 23% yield) as an off-white solid. ¹H NMR (400 MHz, DMSO-d₆) δ 8.92 (dd, J=1.1, 1.1 Hz, 1H), 8.66 (d, J=8.7 Hz, 1H), 8.23 (dd, J=1.4, 1.4 Hz, 1H), 7.88 (dd, J=1.3, 0.8 Hz, 1H), 7.82 (dd, J=1.3, 0.7 Hz, 1H), 7.14 (dd, J=1.4, 0.9 Hz, 1H), 4.07-3.96 (m, 1H), 3.67-3.60 (m, 2H), 2.98 (td, J=12.5, 3.3 Hz, 2H), 2.77 (s, 6H), 2.48 (t, J=0.7 Hz, 3H), 1.87-1.70 (m, 4H). UPLC-MS (Basic 4 min): rt=1.38 min, m/z (ES+) 393.3 [M+H]⁺

### 6-(1H-Imidazol-1-yl)-4-methyl-N-(1-(2-morpholinoacetyl)piperidin-4-yl)picolinamide (19A)

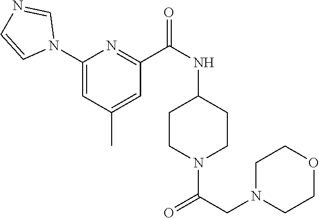 [SMILES: Cc1cc(C(=O)NC2CCN(C(=O)CN3CCOCC3)CC2)nc(-n2ccnc2)c1]

Prepared from 6-(1H-imidazol-1-yl)-4-methyl-N-(piperidin-4-yl)picolinamide hydrochloride 6 and morpholin-4-yl acetic acid according to General Method D to afford the desired product 6-(1H-imidazol-1-yl)-4-methyl-N-(1-(2-morpholinoacetyl)piperidin-4-yl)picolinamide (16 mg, 0.0384 mmol, 31% yield) as a colourless solid. ¹H NMR (400 MHz, DMSO-d₆) δ 8.87 (dq, J=2.4, 1.1 Hz, 1H), 8.59 (dd, J=9.0, 2.8 Hz, 1H), 8.18 (dt, J=3.1, 1.5 Hz, 1H), 7.88-7.75 (m, 2H), 7.11 (dq, J=2.5, 1.2 Hz, 1H), 4.36 (d, J=13.2 Hz, 1H), 4.09 (d, J=13.4 Hz, 2H), 3.55 (p, J=3.1 Hz, 4H), 3.23-2.99 (m, 3H), 2.63 (t, J=12.8 Hz, 1H), 2.48-2.40 (m, 3H), 2.42-2.34 (m, 4H), 1.86-1.43 (m, 4H). UPLC-MS (Basic 4 min): rt=1.15 min, m/z (ES+) 413.3 [M+H]⁺

### 6-(1H-Imidazol-1-yl)-N-(1-(3-methoxypropanoyl)piperidin-4-yl)-4-methylpicolinamide (20A)

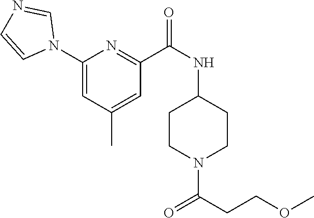 [SMILES: COCCC(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and 3-methoxypropanoic acid according to General Method D to afford the desired product 6-(1H-imidazol-1-yl)-N-(1-(3-methoxypropanoyl)piperidin-4-yl)-4-methylpicolinamide (8.6 mg, 0.0229 mmol, 18% yield) as a colourless solid. ¹H NMR (400 MHz, DMSO-d₆) δ 8.91 (q, J=1.3 Hz, 1H), 8.62 (d, J=8.8 Hz, 1H), 8.22 (h, J=1.6 Hz, 1H), 7.94-7.77 (m, 2H), 7.14 (h, J=1.5 Hz, 1H), 4.44 (d, J=13.1 Hz, 1H), 4.23-4.05 (m, 1H), 3.95 (d, J=13.8 Hz, 1H), 3.56 (ddt, J=6.6, 4.8, 1.6 Hz, 2H), 3.24 (t, J=1.6 Hz, 3H), 3.11 (t, J=13.0 Hz, 1H), 2.74-2.54 (m, 3H), 2.48 (s, 3H), 1.95-1.39 (m, 4H). UPLC-MS (Basic 2 min): rt=0.81 min, m/z (ES+) 372.3 [M+H]⁺

### N-(1-(Dimethylcarbamoyl)piperidin-4-yl)-6-(1H-imidazol-1-yl)-4-methylpicolinamide (21A)

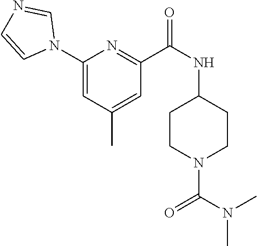 [SMILES: Cc1cc(C(=O)NC2CCN(C(=O)N(C)C)CC2)nc(-n2ccnc2)c1]

Prepared from 6-(1H-imidazol-1-yl)-4-methyl-N-(piperidin-4-yl)picolinamide hydrochloride 6 and dimethyl carbamoyl chloride according to General Method E to afford the desired product N-(1-(dimethylcarbamoyl)piperidin-4-yl)-6-(1H-imidazol-1-yl)-4-methylpicolinamide (19 mg, 0.0541 mmol, 58% yield) as a colourless solid. ¹H NMR (400 MHz, DMSO-d₆) δ 8.93 (dd, J=1.1, 1.1 Hz, 1H), 8.65 (d, J=8.8 Hz, 1H), 8.23 (dd, J=1.4, 1.4 Hz, 1H), 7.87 (dd, J=1.1, 1.1 Hz, 1H), 7.85-7.73 (m, 1H), 7.29-7.00 (m, 1H), 4.09-3.97 (m, 1H), 3.65-3.56 (m, 2H), 2.89-2.76 (m, 2H), 2.75 (s, 6H), 2.47 (s, 3H), 1.77-1.63 (m, 3.5 Hz, 4H). UPLC-MS (Basic 4 min): rt=1.22 min, m/z (ES+) 357.3 [M+H]⁺

### 6-(1H-Imidazol-1-yl)-4-methyl-N-(1-(methylcarbamoyl)piperidin-4-yl)picolinamide (22A)

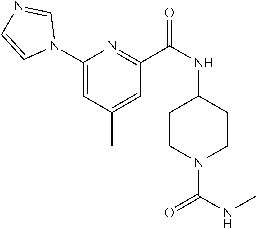 [SMILES: CNC(=O)N1CCC(NC(=O)c2cc(C)cc(-n3ccnc3)n2)CC1]

Prepared from 6-(1H-imidazol-1-yl)-4-methyl-N-(piperidin-4-yl)picolinamide hydrochloride 6 and N-methylaminoformyl chloride according to General Method E to afford the desired product 6-(1H-imidazol-1-yl)-4-methyl-N-(1-(methylcarbamoyl)piperidin-4-yl)picolinamide (16 mg, 0.0471 mmol, 50% yield) as a colourless solid. ¹H NMR (400 MHz, DMSO-d₆) δ 8.92 (dd, J=1.1, 1.1 Hz, 1H), 8.62 (d, J=8.9 Hz, 1H), 8.23 (dd, J=1.4, 1.4 Hz, 1H), 7.87 (dd, J=1.1, 1.1 Hz, 1H), 7.82 (dd, J=1.1, 1.1 Hz, 1H), 7.13 (dd, J=1.2, 1.2 Hz, 1H), 6.48 (d, J=4.4 Hz, 1H), 4.16-3.85 (m, 3H), 2.81-2.70 (m, 2H), 2.57 (d, J=4.3 Hz, 3H), 2.47 (s, 3H), 1.84-1.50 (m, 4H). UPLC-MS (Basic 4 min): rt=1.06 min, m/z (ES+) 343.2 [M+H]⁺

### N-(1,1-Dioxidotetrahydrothiophen-3-yl)-6-(1H-imidazol-1-yl)-4-methylpicolinamide (23A)

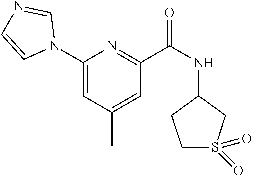 [SMILES: Cc1cc(C(=O)NC2CCS(=O)(=O)C2)nc(-n2ccnc2)c1]

Prepared from cesium 6-(1H-imidazol-1-yl)-4-methylpicolinate 4 and 1,1-dioxothiolan-3-amine hydrochloride according to General Method A to afford the desired product N-(1,1-dioxidotetrahydrothiophen-3-yl)-6-(1H-imidazol-1-yl)-4-methylpicolinamide (15 mg, 0.0468 mmol, 31% yield) as a yellow solid. ¹H NMR (400 MHz, DMSO-d₆) δ 9.00 (d, J=8.5 Hz, 1H), 8.90 (s, 1H), 8.21 (s, 1H), 7.92 (s, 1H), 7.83 (s, 1H), 7.16 (s, 1H), 4.85-4.75 (m, 1H), 3.50 (dd, J=13.1, 8.0 Hz, 2H), 3.46-3.36 (m, 1H), 3.29-3.18 (m, 1H), 2.49 (s, 3H), 2.47-2.39 (m, 1H), 2.37-2.29 (m, 1H). UPLC-MS (Basic 4 min): rt=0.75 min, m/z (ES+) 321.1 [M+H]⁺

### N-(2,2-Dimethyltetrahydro-2H-pyran-4-yl)-6-(1H-imidazol-1-yl)-4-methylpicolinamide (24A)

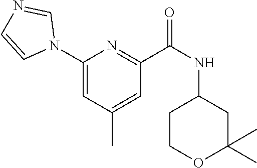 [SMILES: Cc1cc(C(=O)NC2CCOC(C)(C)C2)nc(-n2ccnc2)c1]

Prepared from cesium 6-(1H-imidazol-1-yl)-4-methylpicolinate 4 and 2,2-dimethyltetrahydro-2H-pyran-4-amine according to General Method A to afford the desired product N-(2,2-dimethyltetrahydro-2H-pyran-4-yl)-6-(1H-imidazol-1-yl)-4-methylpicolinamide (8.0 mg, 0.0254 mmol, 19%) as a beige solid. ¹H NMR (400 MHz, DMSO-d₆) δ 8.92 (s, 1H), 8.55 (d, J=8.7 Hz, 1H), 8.23 (t, J=1.4 Hz, 1H), 7.87 (t, J=1.1 Hz, 1H), 7.84-7.79 (m, 1H), 7.17-7.11 (m, 1H), 4.36-4.11 (m, 1H), 3.75-3.58 (m, 2H), 2.48 (d, J=0.7 Hz, 3H), 1.74-1.55 (m, 4H), 1.25-1.15 (m, 6H). UPLC-MS (Basic 4 min): rt=1.36 min, m/z (ES+) 315.2 [M+H]⁺

### N-(4,4-Difluorotetrahydro-2H-pyran-3-yl)-6-(1H-imidazol-1-yl)-4-methylpicolinamide (25A)

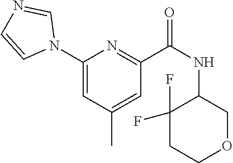 [SMILES: Cc1cc(C(=O)NC2COCCC2(F)F)nc(-n2ccnc2)c1]

Prepared from cesium 6-(1H-imidazol-1-yl)-4-methylpicolinate 4 and 4,4-difluorooxan-3-amine hydrochloride according to General Method B to afford the desired product N-(4,4-difluorotetrahydro-2H-pyran-3-yl)-6-(1H-imidazol-1-yl)-4-methylpicolinamide (14 mg, 0.0418 mmol, 35% yield) as a colourless solid. ¹H NMR (400 MHz, DMSO-d₆) δ 8.88 (s, 1H), 8.61 (d, J=9.6 Hz, 1H), 8.19 (s, 1H), 7.92 (dt, J=1.4, 0.7 Hz, 1H), 7.86 (dt, J=1.3, 0.6 Hz, 1H), 7.16 (s, 1H), 4.61-4.43 (m, 1H), 3.97-3.90 (m, 1H), 3.86 (dt, J=10.0, 4.7 Hz, 1H), 3.71 (ddd, J=11.4, 9.7, 1.6 Hz, 1H), 3.58 (td, J=11.2, 3.1 Hz, 1H), 2.49 (s, 3H), 2.30-2.08 (m, 2H). UPLC-MS (Acidic 4 min): rt=1.45 min, m/z (ES+) 323.1 [M+H]⁺

### N-(5,5-Difluorotetrahydro-2H-pyran-3-yl)-6-(1H-imidazol-1-yl)-4-methylpicolinamide (26A)

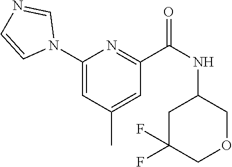 [SMILES: Cc1cc(C(=O)NC2COCC(F)(F)C2)nc(-n2ccnc2)c1]

Prepared from cesium 6-(1H-imidazol-1-yl)-4-methylpicolinate 4 and 5,5-difluorotetrahydro-2H-pyran-3-amine hydrochloride according to General Method B to afford the desired product N-(5,5-difluorotetrahydro-2H-pyran-3-yl)-6-(1H-imidazol-1-yl)-4-methylpicolinamide (14 mg, 0.0417 mmol, 35% yield) as a colourless solid. ¹H NMR (400 MHz, DMSO-d₆) δ 8.85 (s, 1H), 8.67 (d, J=8.8 Hz, 1H), 8.17 (s, 1H), 7.92 (q, J=0.9 Hz, 1H), 7.83 (q, J=0.9 Hz, 1H), 7.17 (s, 1H), 4.29 (qt, J=9.3, 4.8 Hz, 1H), 3.97-3.81 (m, 2H), 3.63 (dt, J=17.7, 11.8 Hz, 1H), 3.48 (dd, J=11.1, 9.3 Hz, 1H), 2.49 (s, 3H), 2.47-2.31 (m, 2H). UPLC-MS (Acidic 4 min): rt=1.05 min, m/z (ES+) 323.1 [M+H]⁺

### N-(3,3-Dimethyltetrahydro-2H-pyran-4-yl)-6-(1H-imidazol-1-yl)-4-methylpicolinamide (27A)

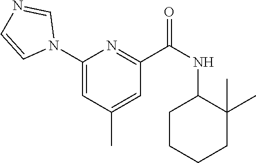 [SMILES: Cc1cc(C(=O)NC2CCCCC2(C)C)nc(-n2ccnc2)c1]

Prepared from lithium 6-(1H-imidazol-1-yl)-4-methylpicolinate 5 and 3,3-dimethyloxan-4-amine hydrochloride according to General Method B to afford the desired product N-(3,3-dimethyltetrahydro-2H-pyran-4-yl)-6-(1H-imidazol-1-yl)-4-methylpicolinamide (32 mg, 0.102 mmol, 71% yield) as an off-white solid. ¹H NMR (400 MHz, ACETONITRILE-d₃) δ 8.42 (d, J=1.2 Hz, 1H), 7.89 (dq, J=1.2, 0.6 Hz, 1H), 7.86-7.78 (m, 2H), 7.59 (dd, J=1.3, 0.8 Hz, 1H), 7.14 (dd, J=1.5, 0.9 Hz, 1H), 4.03 (ddd, J=11.7, 9.9, 4.6 Hz, 1H), 3.98-3.90 (m, 1H), 3.53-3.42 (m, 2H), 3.26-3.19 (m, 1H), 2.50 (t, J=0.9 Hz, 3H), 1.92-1.83 (m, 1H), 1.62 (ddt, J=13.3, 4.8, 2.4 Hz, 1H), 1.06 (d, J=0.7 Hz, 3H), 0.82 (s, 3H). UPLC-MS (Basic 6 min): rt=2.13 min, m/z (ES+) 315.2 [M+H]⁺

### N-(8-Oxabicyclo[3.2.1]octan-3-yl)-6-(1H-imidazol-1-yl)-4-methylpicolinamide 28A

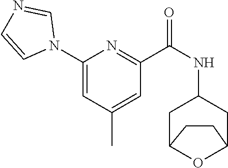 [SMILES: Cc1cc(C(=O)NC2CC3CCC(C2)O3)nc(-n2ccnc2)c1]

Prepared from lithium 6-(1H-imidazol-1-yl)-4-methylpicolinate 4 and 8-oxabicyclo[3.2.1]octan-3-amine according to General Method B to give N-(8-oxabicyclo[3.2.1]octan-3-yl)-6-(1H-imidazol-1-yl)-4-methylpicolinamide (16 mg, 0.0512 mmol, 36% yield) as a colourless solid. ¹H NMR (400 MHz, DMSO-d₆) δ 8.98-8.67 (m, 1H), 8.67-8.40 (m, 1H), 8.28-8.05 (m, 1H), 7.93-7.76 (m, 2H), 7.20-7.11 (m, 1H), 4.42-4.01 (m, 3H), 2.49-2.47 (m, 3H), 2.14-2.03 (m, 3H), 2.03-1.87 (m, 2H), 1.87-1.59 (m, 3H). 77:23 ratio of diastereomers. UPLC-MS (Basic 6 min): rt=1.85 min, m/z (ES+) 313.1 [M+H]⁺, rt=1.90 min, m/z (ES+) 313.1 [M+H]⁺.

### N-(6,6-Dimethyltetrahydro-2H-pyran-3-yl)-6-(1H-imidazol-1-yl)-4-methylpicolinamide (29A)

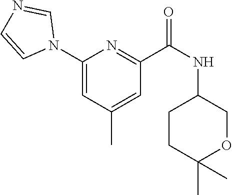 [SMILES: Cc1cc(C(=O)NC2CCC(C)(C)OC2)nc(-n2ccnc2)c1]

Prepared from cesium 6-(1H-imidazol-1-yl)-4-methylpicolinate 4 and (6,6-dimethyltetrahydro-2H-pyran-3-yl)amine according to General Method A to afford the desired product N-(6,6-dimethyltetrahydro-2H-pyran-3-yl)-6-(1H-imidazol-1-yl)-4-methylpicolinamide (1.4 mg, 0.00445 mmol, 3% yield) as a colourless solid. ¹H NMR (400 MHz, DMSO-d₆) δ 8.89 (s, 1H), 8.48 (d, J=8.8 Hz, 1H), 8.20 (s, 1H), 7.91-7.79 (m, 2H), 7.16 (s, 1H), 3.99-3.79 (m, 1H), 3.67-3.44 (m, 2H), 2.49-2.47 (m, 3H), 2.05-1.85 (m, 1H), 1.80-1.70 (m, 1H), 1.66-1.56 (m, 1H), 1.56-1.43 (m, 1H), 1.25 (s, 3H), 1.17 (s, 3H). UPLC-MS (Basic 4 min): rt=1.42 min, m/z (ES+) 315.2 [M+H]⁺

### 6-(1H-Imidazol-1-yl)-4-methyl-N-(2-methyltetrahydro-2H-pyran-4-yl)picolinamide (30A)

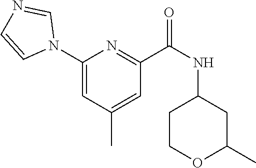 [SMILES: Cc1cc(C(=O)NC2CCOC(C)C2)nc(-n2ccnc2)c1]

Prepared from cesium 6-(1H-imidazol-1-yl)-4-methylpicolinate 4 and 2-methyloxan-4-amine according to General Method A to afford the desired product 6-(1H-imidazol-1-yl)-4-methyl-N-(2-methyltetrahydro-2H-pyran-4-yl)picolinamide (2.1 mg, 0.00699 mmol, 5% yield) as an off-white solid. ¹H NMR (400 MHz, DMSO-d₆) δ 8.99-8.77 (m, 1H), 8.66-8.45 (m, 1H), 8.25-8.12 (m, 1H), 7.89 (dq, J=6.6, 0.9 Hz, 1H), 7.82 (dq, J=1.4, 0.8 Hz, 1H), 7.15 (d, J=6.9 Hz, 1H), 4.37-4.03 (m, 1H), 3.98-3.63 (m, 2H), 3.44 (ddd, J=14.8, 13.3, 7.7 Hz, 1H), 2.48 (dd, J=3.8, 0.7 Hz, 3H), 1.97-1.63 (m, 3H), 1.57-1.33 (m, 1H), 1.12 (dd, J=6.2, 1.9 Hz, 3H). 1:1 ratio of diastereomers. UPLC-MS (Basic 4 min): rt=1.26 min, m/z (ES+) 301.2 [M+H]⁺

### N-(3-Oxabicyclo[3.1.0]hexan-6-yl)-6-(1H-imidazol-1-yl)-4-methylpicolinamide (31A)

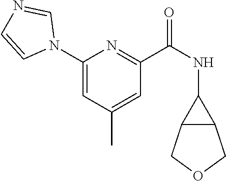 [SMILES: Cc1cc(C(=O)NC2C3COCC32)nc(-n2ccnc2)c1]

Prepared from cesium 6-(1H-imidazol-1-yl)-4-methylpicolinate 4 and 3-oxabicyclo[3.1.0]hexan-6-ylammonium chloride according to General Method A to afford the desired product N-(3-oxabicyclo[3.1.0]hexan-6-yl)-6-(1H-imidazol-1-yl)-4-methylpicolinamide (6.4 mg, 0.0225 mmol, 15% yield) as a colourless solid. ¹H NMR (400 MHz, DMSO-d₆) δ 8.93-8.73 (m, 2H), 8.19 (d, J=1.6 Hz, 1H), 7.92-7.70 (m, 2H), 7.10 (s, 1H), 3.85 (d, 2H), 3.63 (d, J=8.4 Hz, 2H), 2.60-2.51 (m, 1H), 2.44 (d, J=0.7 Hz, 3H), 2.03-1.93 (m, 2H). UPLC-MS (Basic 2 min): rt=1.12 min, m/z (ES+) 285.1 [M+H]⁺

### 6-(1H-Imidazol-1-yl)-4-methyl-N-(tetrahydro-2H-pyran-4-yl)picolinamide (32A)

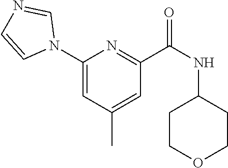 [SMILES: Cc1cc(C(=O)NC2CCOCC2)nc(-n2ccnc2)c1]

Prepared from cesium 6-(1H-imidazol-1-yl)-4-methylpicolinate 4 and 4-aminotetrahydropyran according to General Method A to afford the desired product 6-(1H-imidazol-1-yl)-4-methyl-N-(tetrahydro-2H-pyran-4-yl)picolinamide (26 mg, 0.0908 mmol, 61% yield) as a colourless solid. ¹H NMR (400 MHz, DMSO-d₆) δ 9.01-8.87 (m, 1H), 8.64 (d, J=8.6 Hz, 1H), 8.23 (d, J=1.5 Hz, 1H), 7.98-7.78 (m, 2H), 7.24-7.10 (m, 1H), 4.22-3.84 (m, 3H), 3.40 (td, J=11.5, 2.8 Hz, 2H), 2.48 (s, 3H), 1.83-1.67 (m, 4H). UPLC-MS (Basic 2 min): rt=0.82 min, m/z (ES+) 287.3 [M+H]⁺

### N-Cycloheptyl-6-(1H-imidazol-1-yl)-4-methylpicolinamide (34A)

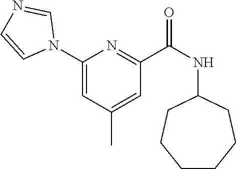 [SMILES: Cc1cc(C(=O)NC2CCCCCC2)nc(-n2ccnc2)c1]

Prepared from cesium 6-(1H-imidazol-1-yl)-4-methylpicolinate 4 and cycloheptylamine according to General Method A to afford the desired product N-cycloheptyl-6-(1H-imidazol-1-yl)-4-methylpicolinamide (6.7 mg, 0.0222 mmol, 18% yield) as a colourless solid. ¹H NMR (400 MHz, DMSO-d₆) δ 8.90 (d, J=1.3 Hz, 1H), 8.55 (d, J=8.6 Hz, 1H), 8.21 (t, J=1.4 Hz, 1H), 7.86 (t, J=1.1 Hz, 1H), 7.80 (t, J=0.8 Hz, 1H), 7.14 (d, J=1.2 Hz, 1H), 4.05-3.91 (m, 1H), 2.47 (s, 3H), 1.89-1.78 (m, 2H), 1.73-1.45 (m, 10H). UPLC-MS (Basic 6 min): rt=2.89 min, m/z (ES+) 299.3 [M+H]⁺

### N-(3,3-Difluorocyclopentyl)-6-(1H-imidazol-1-yl)-4-methylpicolinamide (35A)

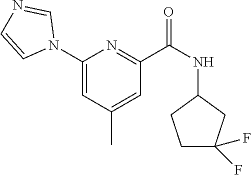 [SMILES: Cc1cc(C(=O)NC2CCC(F)(F)C2)nc(-n2ccnc2)c1]

Prepared from lithium 6-(1H-imidazol-1-yl)-4-methylpicolinate 5 and 3,3-difluorocyclopentan-1-amine hydrochloride according to General Method B to afford the desired product N-(3,3-difluorocyclopentyl)-6-(1H-imidazol-1-yl)-4-methylpicolinamide (32 mg, 0.104 mmol, 54% yield) as a colourless solid. ¹H NMR (400 MHz, DMSO-d₆) δ 8.92 (dd, J=1.1, 1.1 Hz, 1H), 8.83 (d, J=8.3 Hz, 1H), 8.22 (dd, J=1.4, 1.4 Hz, 1H), 7.90 (dd, J=1.1, 1.1 Hz, 1H), 7.82 (d, J=1.0 Hz, 1H), 7.15 (d, J=1.2 Hz, 1H), 4.51 (h, J=8.7 Hz, 1H), 2.60-2.50 (m, 1H), 2.48 (s, 3H), 2.47-2.38 (m, 1H), 2.38-2.25 (m, 1H), 2.21-2.05 (m, 2H), 2.02-1.89 (m, 1H). UPLC-MS (Basic 4 min): rt=1.42 min, m/z (ES+) 307.2 [M+H]⁺

### N-(1,1-Dioxidotetrahydro-2H-thiopyran-3-yl)-6-(1H-imidazol-1-yl)-4-methylpicolinamide (36A)

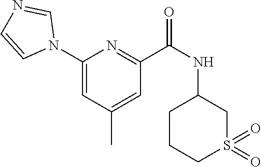 [SMILES: Cc1cc(C(=O)NC2CCCS(=O)(=O)C2)nc(-n2ccnc2)c1]

Prepared from cesium 6-(1H-imidazol-1-yl)-4-methylpicolinate 4 and 1,1-dioxothian-3-amine according to General Method A to afford the desired product N-(1,1-dioxidotetrahydro-2H-thiopyran-3-yl)-6-(1H-imidazol-1-yl)-4-methylpicolinamide (16 mg, 0.0464 mmol, 31% yield) as a yellow solid. ¹H NMR (400 MHz, DMSO-d₆) δ 9.03 (d, J=8.7 Hz, 1H), 8.83 (s, 1H), 8.17 (s, 1H), 7.95-7.90 (m, 1H), 7.85-7.80 (m, 1H), 7.16 (s, 1H), 4.48-4.40 (m, 1H), 3.44-3.33 (m, 2H), 3.22-3.05 (m, 2H), 2.49 (s, 3H), 2.13-2.05 (m, 1H), 1.92-1.84 (m, 2H), 1.83-1.71 (m, 1H). UPLC-MS (Basic 4 min): rt=0.80 min, m/z (ES+) 335.1 [M+H]⁺

### (S)-6-(1H-Imidazol-1-yl)-4-methyl-N-(tetrahydrofuran-3-yl)picolinamide (38A)

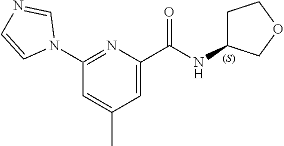

Prepared from cesium 6-(1H-imidazol-1-yl)-4-methylpicolinate 4 and (S)-3-aminotetrahydrofuran hydrochloride according to General Method A to afford the desired product (S)-6-(1H-imidazol-1-yl)-4-methyl-N-(tetrahydrofuran-3-yl)picolinamide (19 mg, 0.068 mmol, 53% yield) as a brown solid. ¹H NMR (400 MHz, DMSO-d₆) δ 8.92 (s, 1H), 8.78 (d, J=7.4 Hz, 1H), 8.23 (s, 1H), 7.91-7.86 (m, 1H), 7.83-7.80 (m, 1H), 7.14 (s, 1H), 4.61-4.48 (m, 1H), 3.96-3.84 (m, 2H), 3.73 (ddd, J=8.3, 7.6, 6.5 Hz, 1H), 3.66 (dd, J=8.8, 5.0 Hz, 1H), 2.48 (s, 3H), 2.25-2.12 (m, 1H), 2.10-1.97 (m, 1H). UPLC-MS (Acidic 4 min): rt=0.88 min, m/z (ES+) 273.2 [M+H]⁺

### (S)-6-(1H-Imidazol-1-yl)-4-methyl-N-(pyrrolidin-3-yl)picolinamide hydrochloride (41a)

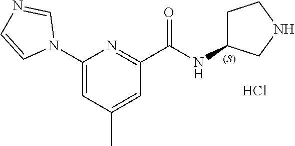

Prepared from tert-butyl (S)-3-(6-(1H-imidazol-1-yl)-4-methylpicolinamido)pyrrolidine-1-carboxylate according to General Method G to afford the desired product (S)-6-(1H-imidazol-1-yl)-4-methyl-N-(pyrrolidin-3-yl)picolinamide hydrochloride (29 mg, 0.0786 mmol, 86% yield) as a pale yellow solid. ¹H NMR (400 MHz, DMSO-d₆) 10.68 (s, 1H), 9.76 (s, 1H), 9.43 (d, J=8.1 Hz, 1H), 9.25 (s, 1H), 8.21-8.16 (m, 1H), 8.07-8.02 (m, 1H), 7.95-7.89 (m, 1H), 4.74 (dd, J=12.6, 6.4 Hz, 1H), 3.51-3.35 (m, 5H), 3.30-2.96 (m, 1H), 2.57-2.53 (m, 3H), 2.34-2.24 (m, 1H), 2.12-2.02 (m, 1H). UPLC-MS (Acidic 4 min): rt=0.40 min, m/z (ES+) 272.2 [M+H]⁺

### (R)-6-(1H-Imidazol-1-yl)-4-methyl-N-(tetrahydrofuran-3-yl)picolinamide (42A)

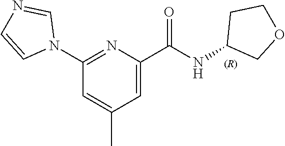

Prepared from cesium 6-(1H-imidazol-1-yl)-4-methylpicolinate 4 and (R)-3-aminotetrahydrofuran hydrochloride according to General Method A to afford the desired product (R)-6-(1H-imidazol-1-yl)-4-methyl-N-(tetrahydrofuran-3-yl)picolinamide (19 mg, 0.068 mmol, 53% yield) as a brown solid. ¹H NMR (400 MHz, DMSO-d₆) δ 8.92 (t, J=1.1 Hz, 1H), 8.77 (d, J=7.4 Hz, 1H), 8.23 (t, J=1.4 Hz, 1H), 7.91-7.86 (m, 1H), 7.84-7.79 (m, 1H), 7.14 (t, J=1.2 Hz, 1H), 4.55 (dtd, J=12.7, 7.4, 5.2 Hz, 1H), 3.90 (ddd, J=17.1, 8.5, 6.4 Hz, 2H), 3.73 (td, J=8.0, 6.5 Hz, 1H), 3.66 (dd, J=8.8, 5.0 Hz, 1H), 2.48 (s, 3H), 2.25-2.12 (m, 1H), 2.10-1.97 (m, 1H). UPLC-MS (Acidic 4 min): rt=0.83 min, m/z (ES+) 273.2 [M+H]⁺

### tert-Butyl (R)-3-(6-(1H-imidazol-1-yl)-4-methylpicolinamido)pyrrolidine-1-carboxylate (43a)

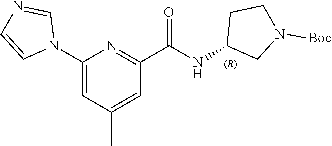

Prepared from cesium 6-(1H-imidazol-1-yl)-4-methylpicolinate 4 and (R)-1-Boc-3-aminopyrrolidine according to General Method A to afford the desired product tert-butyl (R)-3-(6-(1H-imidazol-1-yl)-4-methylpicolinamido)pyrrolidine-1-carboxylate (14 mg, 0.036 mmol, 21% yield) as a colourless gum. ¹H NMR (400 MHz, DMSO-d₆) δ 8.91 (s, 1H), 8.77 (d, J=7.6 Hz, 1H), 8.22 (s, 1H), 7.89 (s, 1H), 7.81 (s, 1H), 7.14 (s, 1H), 5.75 (d, J=2.4 Hz, 1H), 3.57 (d, J=9.4 Hz, 1H), 3.47 (s, 1H), 2.48 (d, J=3.0 Hz, 5H), 2.18-1.94 (m, 2H), 1.40 (s, 9H). UPLC-MS (Acidic 4 min): rt=1.45 min, m/z (ES+) 372.3 [M+H]⁺

### (R)-6-(1H-Imidazol-1-yl)-4-methyl-N-(tetrahydro-2H-pyran-3-yl)picolinamide (44a)

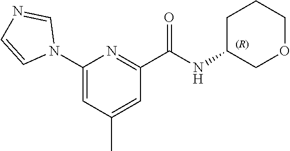

Prepared from cesium 6-(1H-imidazol-1-yl)-4-methylpicolinate 4 and (R)-tetrahydro-2H-pyran-3-amine hydrochloride according to General Method A to afford the desired product (R)-6-(1H-imidazol-1-yl)-4-methyl-N-(tetrahydro-2H-pyran-3-yl)picolinamide (13 mg, 0.045 mmol, 34% yield) as a white solid. ¹H NMR (400 MHz, CHLOROFORM-d) 8.66 (s, 1H), 8.08-7.99 (m, 2H), 7.66 (s, 1H), 7.39 (dd, J=1.4, 0.8 Hz, 1H), 7.29 (s, 1H), 4.19 (dtd, J=9.0, 5.8, 3.1 Hz, 1H), 3.87 (dd, J=11.3, 3.1 Hz, 1H), 3.80-3.56 (m, 4H), 2.56-2.50 (m, 2H), 2.05-1.60 (m, 4H). UPLC-MS (Acidic 4 min): rt=0.96 min m/z (ES+) 287.2 [M+H]⁺

### (S)-6-(1H-Imidazol-1-yl)-4-methyl-N-(tetrahydro-2H-pyran-3-yl)picolinamide (45a)

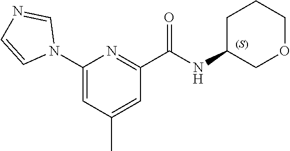

Prepared from cesium 6-(1H-imidazol-1-yl)-4-methylpicolinate 4 and (S)-tetrahydro-2H-pyran-3-amine hydrochloride according to General Method A to afford the desired product (S)-6-(1H-imidazol-1-yl)-4-methyl-N-(tetrahydro-2H-pyran-3-yl)picolinamide (9.3 mg, 0.031 mmol, 26% yield) as a pale orange solid. ¹H NMR (400 MHz, CHLOROFORM-d) 8.45 (s, 1H), 8.08-7.96 (m, 2H), 7.64 (s, 1H), 7.33 (t, J=1.1 Hz, 1H), 4.18 (dtd, J=9.2, 5.6, 2.9 Hz, 1H), 3.87 (dd, J=11.2, 3.1 Hz, 1H), 3.79-3.55 (m, 3H), 2.52 (s, 3H), 1.97 (dd, J=9.1, 4.8 Hz, 1H), 1.82 (ddq, J=18.1, 10.0, 3.3 Hz, 2H), 1.71-1.58 (m, 1H). 1H unobserved. UPLC-MS (Acidic 4 min): rt=0.92 min, m/z (ES+) 287.2 [M+H]⁺

### (S)-6-(1H-imidazol-1-yl)-4-methyl-N-(1-(methylsulfonyl)pyrrolidin-3-yl)picolinamide (46a)

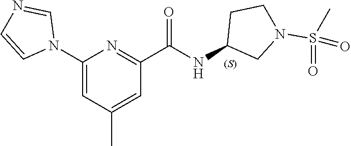

Prepared from (S)-6-(1H-imidazol-1-yl)-4-methyl-N-(pyrrolidin-3-yl)picolinamide hydrochloride according to General Method F to afford the desired product (S)-6-(1H-imidazol-1-yl)-4-methyl-N-(1-(methylsulfonyl)pyrrolidin-3-yl)picolinamide (13 mg, 0.0358 mmol, 49% yield) as a white solid. ¹H NMR (400 MHz, DMSO-d₆) δ 8.90 (s, 1H), 8.80 (d, J=7.5 Hz, 1H), 8.22 (s, 1H), 7.93-7.88 (m, 1H), 7.88-7.80 (m, 1H), 7.15 (s, 1H), 4.66-4.52 (m, 1H), 3.58 (dd, J=10.1, 7.4 Hz, 1H), 3.47 (ddd, J=9.9, 7.9, 4.6 Hz, 1H), 3.38-3.26 (m, 2H), 2.95 (d, J=0.8 Hz, 3H), 2.48 (t, J=0.7 Hz, 3H), 2.25-2.05 (m, 2H). UPLC-MS (Acidic 4 min): rt=0.88 min, m/z (ES+) 350.2 [M+H]⁺

### tert-Butyl (S)-3-(6-(1H-imidazol-1-yl)-4-methylpicolinamido)piperidine-1-carboxylate (47A)

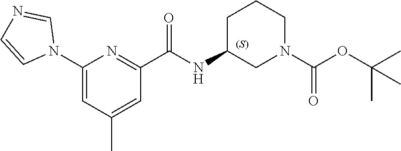

Prepared from tert-butyl (S)-3-(6-chloro-4-methylpicolinamido)piperidine-1-carboxylate according to General Method H to afford the desired product tert-butyl (S)-3-(6-(1H-imidazol-1-yl)-4-methylpicolinamido)piperidine-1-carboxylate (77 mg, 0.192 mmol, 26% yield) as a yellow solid. ¹H NMR (400 MHz, DMSO-d₆) δ 8.87 (s, 1H), 8.55 (d, J=7.8 Hz, 1H), 8.19 (s, 1H), 7.89 (s, 1H), 7.83 (s, 1H), 7.15 (s, 1H), 3.87-3.83 (m, 2H), 3.80-3.72 (m, 1H), 3.03-2.79 (m, 2H), 2.48 (s, 3H), 1.88-1.83 (m, 1H), 1.78-1.67 (m, 2H), 1.47-1.42 (m, 1H), 1.38 (s, 9H). UPLC-MS (Basic 4 min): rt=1.64 min, m/z (ES+) 386.3 [M+H]⁺

### (S)-6-(1H-Imidazol-1-yl)-4-methyl-N-(piperidin-3-yl)picolinamide dihydrochloride (48A)

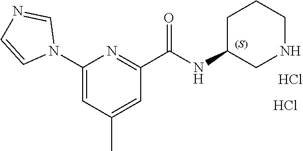

Prepared from tert-butyl (S)-3-(6-(1H-imidazol-1-yl)-4-methylpicolinamido)piperidine-1-carboxylate according to General Method G to afford the desired product (S)-6-(1H-imidazol-1-yl)-4-methyl-N-(piperidin-3-yl)picolinamide dihydrochloride (65 mg, 0.176 mmol, 97% yield) as a yellow solid. ¹H NMR (400 MHz, DMSO-d₆) δ 10.58 (s, 1H), 9.43 (s, 1H), 9.23 (d, J=8.7 Hz, 1H), 9.05-8.97 (m, 1H), 8.78 (s, 1H), 8.18 (s, 1H), 8.04 (s, 1H), 7.93 (s, 1H), 4.39-4.30 (m, 1H), 3.32-3.08 (m, 4H), 2.86-2.79 (m, 1H), 2.53 (s, 3H), 1.98-1.67 (m, 4H). UPLC-MS (Basic 4 min): rt=1.02 min, m z (ES+) 286.2 [M+H]⁺

### tert-Butyl (R)-3-(6-(1H-imidazol-1-yl)-4-methylpicolinamido)piperidine-1-carboxylate (49A)

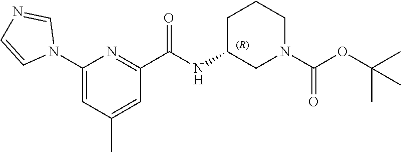

Prepared from cesium 6-(1H-imidazol-1-yl)-4-methylpicolinate 4 and (R)-1-Boc-3-piperidinamine according to General Method A to afford the desired product tert-butyl (R)-3-(6-(1H-imidazol-1-yl)-4-methylpicolinamido)piperidine-1-carboxylate (24 mg, 0.0616 mmol, 29% yield) as an off-white solid. ¹H NMR (400 MHz, DMSO-d₆) δ 9.70 (s, 1H), 8.63 (d, J=8.2 Hz, 1H), 8.57 (s, 1H), 8.04 (s, 1H), 7.97 (s, 1H), 7.70 (s, 1H), 4.00-3.79 (m, 3H), 3.00-2.74 (m, 2H), 2.53 (s, 3H), 1.94-1.87 (m, 1H), 1.78-1.68 (m, 2H), 1.49-1.36 (m, 10H). UPLC-MS (Basic 4 min): rt=1.64 min, m/z (ES+) 386.4 [M+H]⁺

### (S)-6-(1H-Imidazol-1-yl)-4-methyl-N-(1-(methylsulfonyl)piperidin-3-yl)picolinamide (50A)

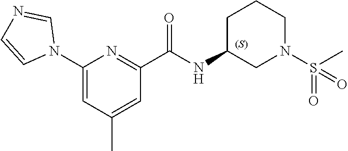

Prepared from (S)-6-(1H-imidazol-1-yl)-4-methyl-N-(piperidin-3-yl)picolinamide dihydrochloride according to General Method F to afford the desired product (S)-6-(1H-imidazol-1-yl)-4-methyl-N-(1-(methylsulfonyl)piperidin-3-yl)picolinamide (20 mg, 0.0539 mmol, 33% yield) as an off-white solid. ¹H NMR (400 MHz, DMSO-d₆) δ 8.87 (s, 1H), 8.62 (d, J=8.4 Hz, 1H), 8.19 (s, 1H), 7.93-7.88 (m, 1H), 7.86-7.81 (m, 1H), 7.15 (s, 1H), 4.08-3.96 (m, 1H), 3.58 (dd, J=10.5, 3.5 Hz, 1H), 3.50-3.43 (m, 1H), 2.91 (s, 3H), 2.86-2.72 (m, 2H), 2.49 (s, 3H), 1.91-1.55 (m, 4H). UPLC-MS (Basic 4 min): rt=1.21 min, m/z (ES+) 364.3 [M+H]⁺.

### (R)-6-(1H-Imidazol-1-yl)-4-methyl-N-(piperidin-3-yl)picolinamide dihydrochloride (51A)

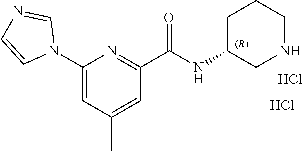

Prepared from tert-butyl (R)-3-(6-(1H-imidazol-1-yl)-4-methylpicolinamido)piperidine-1-carboxylate according to General Method G to afford the desired product (R)-6-(1H-imidazol-1-yl)-4-methyl-N-(piperidin-3-yl)picolinamide dihydrochloride (18 mg, 0.050 mmol, 97% yield) as a yellow solid. ¹H NMR (400 MHz, DMSO-d₆) δ 10.38 (s, 1H), 9.31-9.26 (m, 1H), 9.14 (d, J=8.6 Hz, 1H), 8.93-8.88 (m, 1H), 8.73 (s, 1H), 8.15 (s, 1H), 8.02 (s, 1H), 7.87 (s, 1H), 4.35-4.28 (m, 1H), 3.33-3.22 (m, 3H), 3.17-3.07 (m, 1H), 2.86-2.79 (m, 1H), 2.54 (s, 3H), 1.96-1.70 (m, 4H). UPLC-MS (Basic 4 min): rt=1.05 min, m/z (ES+) 286.2 [M+H]⁺

### (R)-6-(1H-Imidazol-1-yl)-4-methyl-N-(pyrrolidin-3-yl)picolinamide dihydrochloride (52A)

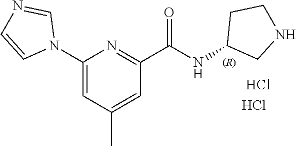

Prepared from tert-butyl (R)-3-(6-(1H-imidazol-1-yl)-4-methylpicolinamido)pyrrolidine-1-carboxylate according to General Method G to afford the desired product (R)-6-(1H-imidazol-1-yl)-4-methyl-N-(pyrrolidin-3-yl)picolinamide dihydrochloride (29 mg, 0.078 mmol, 99% yield) as a yellow solid. ¹H NMR (400 MHz, DMSO-d₆) δ 10.63 (s, 1H), 9.69 (s, 1H), 9.38 (d, J=8.0 Hz, 1H), 9.16 (s, 1H), 8.79 (s, 1H), 8.14 (s, 1H), 8.01 (d, J=1.1 Hz, 1H), 7.89 (s, 1H), 4.74-4.68 (m, 1H), 3.36 (d, J=6.3 Hz, 3H), 3.25-3.17 (m, 2H), 2.51 (s, 3H), 2.24 (dt, J=14.8, 7.4 Hz, 1H), 2.04 (dt, J=13.2, 6.7 Hz, 1H). UPLC-MS (Basic 4 min): rt=0.96 min, m/z (ES+) 272.3 [M+H]⁺

### (R)-6-(1H-Imidazol-1-yl)-4-methyl-N-(1-(methylsulfonyl)pyrrolidin-3-yl)picolinamide (53A)

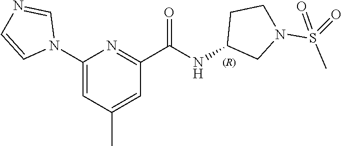

Prepared from (R)-6-(1H-imidazol-1-yl)-4-methyl-N-(pyrrolidin-3-yl)picolinamide hydrochloride according to General Method F to afford the desired product (R)-6-(1H-imidazol-1-yl)-4-methyl-N-(1-(methylsulfonyl)pyrrolidin-3-yl)picolinamide (17 mg, 0.047 mmol, 58% yield) as a white solid. ¹H NMR (400 MHz, DMSO-d₆) 8.90 (d, J=1.4 Hz, 1H), 8.80 (d, J=7.5 Hz, 1H), 8.21 (t, J=1.4 Hz, 1H), 7.90 (t, J=1.1 Hz, 1H), 7.82 (t, J=0.9 Hz, 1H), 7.15 (t, J=1.1 Hz, 1H), 4.59 (h, J=7.3 Hz, 1H), 3.58 (dd, J=10.1, 7.4 Hz, 1H), 3.47 (ddd, J=9.9, 8.0, 4.6 Hz, 1H), 3.31-3.25 (m, 2H), 2.95 (s, 3H), 2.48 (s, 3H), 2.26-2.05 (m, 2H). UPLC-MS (Acidic 4 min): rt=1.12 min, m/z (ES+) 350.1 [M+H]⁺

### (R)-6-(1H-Imidazol-1-yl)-4-methyl-N-(1-(methylsulfonyl)piperidin-3-yl)picolinamide (54A)

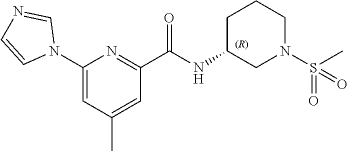

Prepared from (R)-6-(1H-imidazol-1-yl)-4-methyl-N-(piperidin-3-yl)picolinamide dihydrochloride according to General Method F to afford the desired product (R)-6-(1H-imidazol-1-yl)-4-methyl-N-(1-(methylsulfonyl)piperidin-3-yl)picolinamide (20 mg, 0.0545 mmol, 65% yield) as a white solid. ¹H NMR (400 MHz, DMSO-d₆) δ 8.87 (t, J=1.1 Hz, 1H), 8.61 (d, J=8.4 Hz, 1H), 8.19 (t, J=1.4 Hz, 1H), 7.93-7.88 (m, 1H), 7.86-7.81 (m, 1H), 7.15 (t, J=1.1 Hz, 1H), 4.09-3.95 (m, 1H), 3.58 (dd, J=11.2, 4.2 Hz, 1H), 3.50-3.43 (m, 1H), 2.91 (s, 3H), 2.86-2.72 (m, 2H), 2.48 (t, J=0.7 Hz, 3H), 1.91-1.79 (m, 2H), 1.79-1.67 (m, 1H), 1.70-1.54 (m, 1H). UPLC-MS (Basic 4 min): rt=1.21 min, m/z (ES+) 364.3 [M+H]⁺

### (S)—N-(1,1-Dioxidotetrahydrothiophen-3-yl)-6-(1H-imidazol-1-yl)-4-methylpicolinamide (55A)

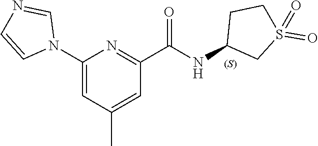

Prepared from lithium 6-(1H-imidazol-1-yl)-4-methylpicolinate 5 and (3S)-1,1-dioxothiolan-3-amine hydrochloride according to General Method B to afford the desired product (S)—N-(1,1-dioxidotetrahydrothiophen-3-yl)-6-(1H-imidazol-1-yl)-4-methylpicolinamide (20 mg, 0.062 mmol, 52% yield) as a white solid. ¹H NMR (400 MHz, DMSO-d₆) δ 8.99 (d, J=8.4 Hz, 1H), 8.90 (s, 1H), 8.21 (s, 1H), 7.94-7.89 (m, 1H), 7.86-7.81 (m, 1H), 7.16 (s, 1H), 4.88-4.73 (m, 1H), 3.55-3.46 (m, 1H), 3.46-3.35 (m, 1H), 3.31-3.18 (m, 2H), 2.49 (s, 3H), 2.47-2.26 (m, 2H). UPLC-MS (Basic 4 min): rt=1.08 min, m/z (ES+) 321.1 [M+H]⁺

### (R)-6-(1H-Imidazol-1-yl)-4-methyl-N-(1-methyl-2-oxopiperidin-3-yl)picolinamide (56A)

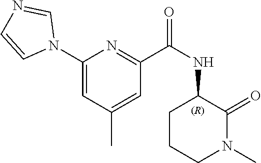

Prepared from lithium 6-(1H-imidazol-1-yl)-4-methylpicolinate 5 and (R)-3-amino-1-methylpiperidin-2-one hydrochloride according to General Method B to afford the desired product (R)-6-(1H-imidazol-1-yl)-4-methyl-N-(1-methyl-2-oxopiperidin-3-yl)picolinamide (21 mg, 0.067 mmol, 35% yield) as a white solid. ¹H NMR (400 MHz, DMSO-d₆) δ 9.09 (d, J=8.5 Hz, 1H), 8.92-8.87 (m, 1H), 8.23 (t, J=1.4 Hz, 1H), 7.94-7.89 (m, 1H), 7.89-7.81 (m, 1H), 7.19-7.12 (m, 1H), 4.52-4.41 (m, 1H), 3.42-3.34 (m, 2H), 2.88 (s, 3H), 2.50 (s, 3H), 2.11-1.86 (m, 4H). UPLC-MS (Basic 4 min): rt=1.11 min m/z (ES+) 314.2 [M+H]⁺

### (R)—N-(1,1-Dioxidotetrahydro-2H-thiopyran-3-yl)-6-(1H-imidazol-1-yl)-4-methylpicolinamide (57A)

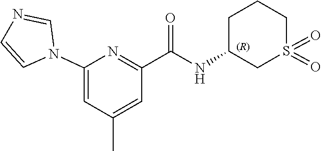

Prepared from lithium 6-(1H-imidazol-1-yl)-4-methylpicolinate 5 and (3R)-1,1-dioxothian-3-amine according to General Method B to afford the desired product (R)—N-(1,1-dioxidotetrahydro-2H-thiopyran-3-yl)-6-(1H-imidazol-1-yl)-4-methylpicolinamide (15 mg, 0.045 mmol, 37% yield) as a white solid. ¹H NMR (400 MHz, ACETONITRILE-d₃) δ 8.84 (s, 1H), 8.49 (d, J=1.2 Hz, 1H), 7.90-7.85 (m, 2H), 7.61 (dq, J=1.4, 0.7 Hz, 1H), 7.17-7.11 (m, 1H), 4.61 (ddq, J=11.6, 7.7, 3.9 Hz, 1H), 3.37 (ddd, J=14.0, 4.0, 1.8 Hz, 1H), 3.17 (ddd, J=14.1, 7.8, 2.0 Hz, 1H), 3.13-3.00 (m, 2H), 2.50 (t, J=0.7 Hz, 3H), 2.24-2.16 (m, 1H), 2.07-1.96 (m, 1H), 1.92-1.75 (m, 2H). UPLC-MS (Basic 6 min): rt=1.65 min, m/z (ES+) 335.1 [M+H]⁺

### (R)-6-(1H-Imidazol-1-yl)-4-methyl-N-(2-oxopiperidin-3-yl)picolinamide (58A)

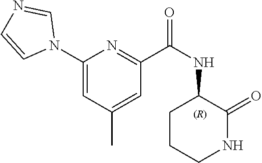

Prepared from lithium 6-(1H-imidazol-1-yl)-4-methylpicolinate 5 and (R)-3-aminopiperidin-2-one according to General Method B to afford the desired product (R)-6-(1H-imidazol-1-yl)-4-methyl-N-(2-oxopiperidin-3-yl)picolinamide (20 mg, 0.066 mmol, 34% yield) as an off-white solid. ¹H NMR (400 MHz, DMSO-d₆) δ 9.07 (d, J=8.6 Hz, 1H), 8.88 (dd, J=1.0, 1.0 Hz, 1H), 8.22 (dq, J=1.4, 0.5 Hz, 1H), 7.90 (dq, J=1.4, 0.7 Hz, 1H), 7.82 (dd, J=1.4, 0.8 Hz, 1H), 7.68 (s, 1H), 7.15 (dd, J=1.5, 0.7 Hz, 1H), 4.40 (ddd, J=11.2, 8.5, 6.3 Hz, 1H), 3.43-3.36 (m, 2H), 2.49 (s, 3H), 2.07-1.74 (m, 4H). UPLC-MS (Basic 4 min): rt=1.04 min, m/z (ES+) 300.1 [M+H]⁺.

### (R)-6-(1H-Imidazol-1-yl)-4-methyl-N-(1-methylpiperidin-3-yl)picolinamide (59A)

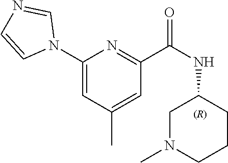

Prepared from lithium 6-(1H-imidazol-1-yl)-4-methylpicolinate 5 and (3R)-1-methyl-3-piperidinamine dihydrochloride according to General Method B to afford the desired product (R)-6-(1H-imidazol-1-yl)-4-methyl-N-(1-methylpiperidin-3-yl)picolinamide (8.2 mg, 0.027 mmol, 14% yield) as a white solid. ¹H NMR (400 MHz, DMSO-d₆) δ 8.85 (t, J=1.1 Hz, 1H), 8.50 (d, J=8.8 Hz, 1H), 8.17 (t, J=1.4 Hz, 1H), 7.87 (t, J=1.1 Hz, 1H), 7.81 (t, J=1.0 Hz, 1H), 7.14 (dd, J=1.5, 0.9 Hz, 1H), 4.06-3.93 (m, 1H), 2.74-2.54 (m, 2H), 2.47 (s, 3H), 2.18 (s, 3H), 2.10-1.86 (m, 2H), 1.80-1.61 (m, 2H), 1.60-1.41 (m, 2H). UPLC-MS (Basic 4 min): rt=1.24 min, m/z (ES+) 300.2 [M+H]⁺

### (S)-6-(1H-Imidazol-1-yl)-4-methyl-N-(2-oxopiperidin-3-yl)picolinamide (60A)

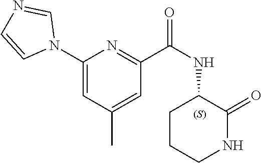

Prepared from lithium 6-(1H-imidazol-1-yl)-4-methylpicolinate 5 and (S)-3-aminopiperidin-2-one according to General Method B to afford the desired product (S)-6-(1H-imidazol-1-yl)-4-methyl-N-(2-oxopiperidin-3-yl)picolinamide (29 mg, 0.096 mmol, 50% yield) as a white solid. ¹H NMR (400 MHz, DMSO-d₆) δ 9.07 (d, J=8.6 Hz, 1H), 8.88 (dd, J=1.1, 1.1 Hz, 1H), 8.22 (dd, J=1.4, 1.4 Hz, 1H), 7.90 (dd, J=1.3, 0.8 Hz, 1H), 7.82 (dd, J=1.3, 0.7 Hz, 1H), 7.68 (s, 1H), 7.15 (dd, J=1.5, 0.9 Hz, 1H), 4.40 (ddd, J=11.3, 8.6, 6.3 Hz, 1H), 3.22-3.17 (dd, m, 2H), 2.49 (s, 3H), 2.08-1.71 (m, 4H). UPLC-MS (Basic 4 min): rt=1.04 min, m z (ES+) 300.1 [M+H]⁺

### 2-(1H-Imidazol-1-yl)-6-methyl-N-(1-(methylsulfonyl)piperidin-4-yl)pyrimidine-4-carboxamide (61A)

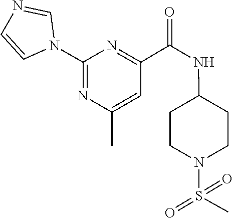 [SMILES: Cc1cc(C(=O)NC2CCN(S(C)(=O)=O)CC2)nc(-n2ccnc2)n1]

Prepared from lithium 2-(1H-imidazol-1-yl)-6-methylpyrimidine-4-carboxylate 8 and 1-(methylsulfonyl)-4-piperidinamine according to General Method B to afford the desired product 2-(1H-imidazol-1-yl)-6-methyl-N-(1-(methylsulfonyl)piperidin-4-yl)pyrimidine-4-carboxamide (26 mg, 0.072 mmol, 36% yield) as a white solid. ¹H NMR (400 MHz, DMSO-d₆) δ 8.93 (dd, J=1.1 Hz, 1H), 8.91 (s, 1H), 8.19 (dd, J=1.4 Hz, 1H), 7.84 (d, J=0.5 Hz, 1H), 7.16 (dd, J=1.5, 0.9 Hz, 1H), 4.00 (dd, J=8.6, 4.2 Hz, 1H), 3.63 (d, J=11.8 Hz, 2H), 2.90 (s, 3H), 2.89-2.78 (m, 2H), 2.63 (s, 3H), 1.93-1.72 (m, 4H). UPLC-MS (Basic 4 min): rt=1.25 min, m/z (ES+) 365.2 [M+H]⁺

### (R)-6-Bromo-N-(tetrahydro-2H-pyran-3-yl)-4-(trifluoromethyl)picolinamide

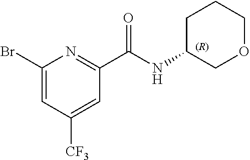

Prepared from 6-bromo-4-(trifluoromethyl)picolinic acid and (R)-tetrahydro-2H-pyran-3-amine hydrochloride according to General Method B to afford the desired product (R)-6-bromo-N-(tetrahydro-2H-pyran-3-yl)-4-(trifluoromethyl)picolinamide (559 mg, 1.56 mmol, 67% yield) as a white solid. ¹H NMR (400 MHz, DMSO-d₆) δ 8.62 (d, J=8.5 Hz, 1H), 8.41-8.36 (m, 1H), 8.24-8.19 (m, 1H), 4.02-3.89 (m, 1H), 3.78-3.69 (m, 2H), 3.38-3.31 (m, 2H), 1.92-1.50 (m, 4H).

### (R)-6-(1H-imidazol-1-yl)-N-(tetrahydro-2H-pyran-3-yl)-4-(trifluoromethyl)picolinamide (62A)

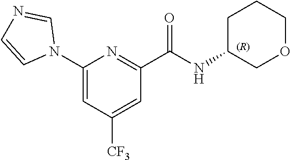

Prepared from (R)-6-bromo-N-(tetrahydro-2H-pyran-3-yl)-4-(trifluoromethyl)picolinamide according to General Method H to afford the desired product (R)-6-(1H-imidazol-1-yl)-N-(tetrahydro-2H-pyran-3-yl)-4-(trifluoromethyl)picolinamide (415 mg, 1.22 mmol, 77% yield) as an off-white solid. ¹H NMR (400 MHz, DMSO-d₆) δ 9.07 (t, J=1.1 Hz, 1H), 8.72 (d, J=8.7 Hz, 1H), 8.47-8.44 (m, 1H), 8.41 (t, J=1.4 Hz, 1H), 8.14-8.09 (m, 1H), 7.22-7.17 (m, 1H), 4.08-3.94 (m, 1H), 3.84-3.75 (m, 2H), 3.38-3.27 (m, 2H), 1.98-1.55 (m, 4H). UPLC-MS (Basic 4 min): rt=1.18 min m/z (ES+) 341.1 [M+H]⁺

### (R)—N-(2,6-Dioxopiperidin-3-yl)-6-(1H-imidazol-1-yl)-4-methylpicolinamide (63A)

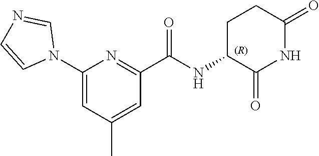

Prepared from lithium 6-(1H-imidazol-1-yl)-4-methylpicolinate 5 and (3R)-3-aminopiperidine-2,6-dione hydrochloride according to General Method A to afford the desired product (R)—N-(2,6-dioxopiperidin-3-yl)-6-(1H-imidazol-1-yl)-4-methylpicolinamide (9.0 mg, 0.028 mmol, 15% yield) as a white solid. ¹H NMR (400 MHz, ACETONITRILE-d₃) δ 8.81 (s, 1H), 8.58-8.41 (m, 2H), 7.92-7.89 (m, 1H), 7.87 (dt, J=2.5, 1.6 Hz, 1H), 7.65-7.60 (m, 1H), 7.17-7.11 (m, 1H), 4.79 (ddd, J=13.3, 8.4, 5.8 Hz, 1H), 2.83-2.63 (m, 2H), 2.53-2.49 (m, 3H), 2.36-2.22 (m, 2H). UPLC-MS (Basic 6 min): rt=1.43 min m/z (ES+) 314.1 [M+H]⁺

### (R)—N-(6,6-Dimethyltetrahydro-2H-pyran-3-yl)-6-(1H-imidazol-1-yl)-4-methylpicolinamide (64A)

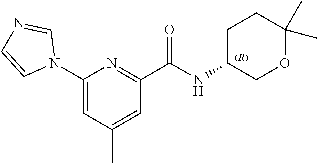

Isolated by chiral separation of N-(6,6-dimethyltetrahydro-2H-pyran-3-yl)-6-(1H-imidazol-1-yl)-4-methylpicolinamide according to Purification Method A to afford the desired product (R)—N-(6,6-dimethyltetrahydro-2H-pyran-3-yl)-6-(1H-imidazol-1-yl)-4-methylpicolinamide (51 mg, 0.162 mmol, 36% yield) as a white solid. ¹H NMR (400 MHz, ACETONE-d₆) δ 8.54 (dd, J=1.1, 1.1 Hz, 1H), 8.31 (d, J=8.6 Hz, 1H), 7.93 (dd, J=1.4, 1.4 Hz, 1H), 7.90 (dq, J=1.5, 0.7 Hz, 1H), 7.79 (dq, J=1.5, 0.8 Hz, 1H), 7.12 (dd, J=1.5, 0.9 Hz, 1H), 4.02 (ddd, J=8.5, 5.5, 4.2 Hz, 1H), 3.77 (ddd, J=11.4, 4.3, 1.0 Hz, 1H), 3.60-3.27 (m, 1H), 2.53 (t, J=0.7 Hz, 3H), 1.96-1.88 (m, 2H), 1.74-1.65 (m, 1H), 1.62-1.44 (m, 1H), 1.24 (s, 3H), 1.21 (s, 3H). UPLC-MS (Basic 4 min): rt=1.37 min, m/z (ES+) 315.3 [M+H]⁺

### (S)—N-(6,6-Dimethyltetrahydro-2H-pyran-3-yl)-6-(1H-imidazol-1-yl)-4-methylpicolinamide (65A)

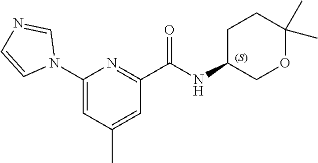

Isolated by chiral separation of N-(6,6-dimethyltetrahydro-2H-pyran-3-yl)-6-(1H-imidazol-1-yl)-4-methylpicolinamide according to Purification Method A to afford the desired product (S)—N-(6,6-dimethyltetrahydro-2H-pyran-3-yl)-6-(1H-imidazol-1-yl)-4-methylpicolinamide (50 mg, 0.160 mmol, 35% yield) as a white solid. ¹H NMR (400 MHz, ACETONE-d₆) δ 8.54 (s, 1H), 8.31 (d, J=8.5 Hz, 1H), 7.93 (dd, J=1.4, 1.4 Hz, 1H), 7.90 (dq, J=1.6, 0.7 Hz, 1H), 7.79 (dq, J=1.6, 0.7 Hz, 1H), 7.12 (dd, J=1.5, 0.9 Hz, 1H), 4.02 (ddd, J=8.5, 5.6, 4.4 Hz, 1H), 3.77 (ddd, J=11.3, 4.3, 1.0 Hz, 1H), 3.57 (dd, J=11.4, 8.4 Hz, 1H), 2.54 (t, J=0.7 Hz, 3H), 1.96-1.85 (m, 2H), 1.69 (ddd, J=13.6, 5.7, 4.7 Hz, 1H), 1.57 (ddd, J=13.7, 9.2, 5.8 Hz, 1H), 1.24 (s, 3H), 1.21 (s, 3H). UPLC-MS (Basic 4 min): rt=1.37 min, m z (ES+) 315.3 [M+H]⁺

### (R)—N-(5,5-Difluorotetrahydro-2H-pyran-3-yl)-6-(1H-imidazol-1-yl)-4-methylpicolinamide (66A)

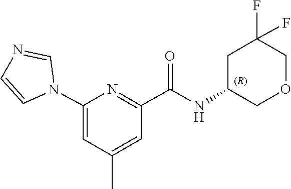

Isolated by chiral separation of N-(5,5-difluorotetrahydro-2H-pyran-3-yl)-6-(1H-imidazol-1-yl)-4-methylpicolinamide according to Purification Method B to afford the desired product (R)—N-(5,5-difluorotetrahydro-2H-pyran-3-yl)-6-(1H-imidazol-1-yl)-4-methylpicolinamide (59 mg, 0.183 mmol, 31% yield) as a white solid. ¹H NMR (400 MHz, DMSO-d₆) δ 8.68 (d, J=8.8 Hz, 1H), 8.20-8.14 (m, 1H), 7.92 (s, 1H), 7.86-7.81 (m, 1H), 7.19-7.14 (m, 1H), 4.36-4.22 (m, 1H), 3.98-3.84 (m, 2H), 3.69-3.54 (m, 1H), 3.52-3.42 (m, 1H), 2.49-2.47 (m, 3H), 2.47-2.29 (m, 2H). 1H unobserved. UPLC-MS (Basic 4 min): rt=1.37 min, m/z (ES+) 315.3 [M+H]⁺

### (R)—N-(5,5-Ddifluorotetrahydro-2H-pyran-3-yl)-6-(1H-imidazol-1-yl)-4-methylpicolinamide (67A)

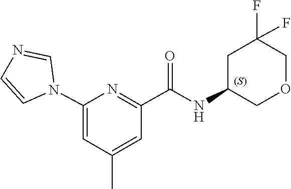

Isolated by chiral separation of N-(5,5-difluorotetrahydro-2H-pyran-3-yl)-6-(1H-imidazol-1-yl)-4-methylpicolinamide according to Purification Method B to afford the desired product (R)—N-(5,5-difluorotetrahydro-2H-pyran-3-yl)-6-(1H-imidazol-1-yl)-4-methylpicolinamide (52 mg, 0.160 mmol, 27% yield) as a white solid. ¹H NMR (400 MHz, DMSO-d₆) δ 8.88-8.81 (m, 1H), 8.68 (dd, J=9.0, 2.4 Hz, 1H), 8.20-8.13 (m, 1H), 7.92 (dd, J=2.3, 1.3 Hz, 1H), 7.86-7.81 (m, 1H), 7.19-7.13 (m, 1H), 4.34-4.23 (m, 1H), 3.98-3.82 (m, 2H), 3.70-3.55 (m, 1H), 3.52-3.42 (m, 1H), 2.48 (s, 3H), 2.47-2.30 (m, 2H). UPLC-MS (Basic 4 min): rt=1.37 min, m/z (ES+) 315.3 [M+H]⁺

### (R)—N-(1,1-Dioxidotetrahydrothiophen-3-yl)-2-(1H-imidazol-1-yl)-6-methylpyrimidine-4-carboxamide (68A)

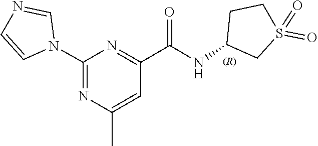

Prepared from lithium 2-(1H-imidazol-1-yl)-6-methylpyrimidine-4-carboxylate 8 and (3R)-1,1-dioxothiolan-3-amine hydrochloride according to General Method B to afford the desired product (R)—N-(1,1-dioxidotetrahydrothiophen-3-yl)-2-(1H-imidazol-1-yl)-6-methylpyrimidine-4-carboxamide (16 mg, 0.049 mmol, 26% yield) as a white solid. ¹H NMR (400 MHz, DMSO-d₆) δ 9.23 (d, J=8.3 Hz, 1H), 8.90 (d, J=1.2 Hz, 1H), 8.17 (dd, J=1.5, 1.5 Hz, 1H), 7.86 (s, 1H), 7.18 (dd, J=1.3, 1.3 Hz, 1H), 4.89-4.72 (m, 1H), 3.53 (dd, J=13.2, 8.1 Hz, 1H), 3.49-3.38 (m, 1H), 3.31-3.20 (m, 2H), 2.64 (s, 3H), 2.48-2.40 (m, 1H), 2.39-2.26 (m, 1H). UPLC-MS (Basic 4 min): rt=1.07 min, m/z (ES+) 322.1 [M+H]⁺

### (R)-2-(1H-Imidazol-1-yl)-6-methyl-N-(tetrahydro-2H-pyran-3-yl)pyrimidine-4-carboxamide (69A)

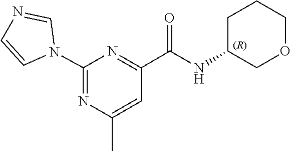

Prepared from lithium 2-(1H-imidazol-1-yl)-6-methylpyrimidine-4-carboxylate 8 and (R)-tetrahydro-2H-pyran-3-amine hydrochloride according to General Method B to afford the desired product (R)-2-(1H-imidazol-1-yl)-6-methyl-N-(tetrahydro-2H-pyran-3-yl)pyrimidine-4-carboxamide (11 mg, 0.039 mmol, 20% yield) as a white solid. ¹H NMR (400 MHz, DMSO-d₆) δ 8.92 (t, J=1.2 Hz, 1H), 8.77 (d, J=8.6 Hz, 1H), 8.18 (t, J=1.4 Hz, 1H), 7.83 (d, J=0.6 Hz, 1H), 7.16 (t, J=1.3 Hz, 1H), 4.06-3.92 (m, 1H), 3.83-3.69 (m, 2H), 3.32-3.25 (m, 2H), 2.63 (s, 3H), 1.93 (d, J=12.1 Hz, 1H), 1.84-1.47 (m, 3H). UPLC-MS (Basic 4 min): rt=1.22 min, m/z (ES+) 288.2 [M+H]⁺

### (S)—N-(4,4-Difluorotetrahydro-2H-pyran-3-yl)-6-(1H-imidazol-1-yl)-4-methylpicolinamide (70A)

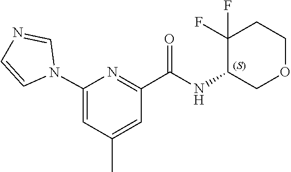

Isolated by chiral separation of N-(4,4-difluorotetrahydro-2H-pyran-3-yl)-6-(1H-imidazol-1-yl)-4-methylpicolinamide according to Purification Method C to afford the desired product (S)—N-(4,4-difluorotetrahydro-2H-pyran-3-yl)-6-(1H-imidazol-1-yl)-4-methylpicolinamide (37 mg, 0.113 mmol, 27% yield) as a white solid. ¹H NMR (400 MHz, DMSO-d₆) δ 8.90-8.85 (m, 1H), 8.61 (d, J=9.7 Hz, 1H), 8.21-8.16 (m, 1H), 7.95-7.90 (m, 1H), 7.88-7.83 (m, 1H), 7.18-7.13 (m, 1H), 4.61-4.43 (m, 1H), 3.97-3.90 (m, 1H), 3.89-3.82 (m, 1H), 3.71 (ddd, J=11.5, 9.8, 1.6 Hz, 1H), 3.62-3.54 (m, 1H), 2.49 (s, 3H), 2.34-2.09 (m, 2H). UPLC-MS (Basic 4 min): rt=1.26 min, m/z (ES+) 323.1 [M+H]⁺

### (R)—N-(4,4-Difluorotetrahydro-2H-pyran-3-yl)-6-(1H-imidazol-1-yl)-4-methylpicolinamide (71A)

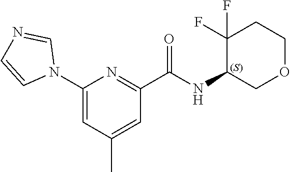

Isolated by chiral separation of N-(4,4-difluorotetrahydro-2H-pyran-3-yl)-6-(1H-imidazol-1-yl)-4-methylpicolinamide according to Purification Method C to afford the desired product (R)—N-(4,4-difluorotetrahydro-2H-pyran-3-yl)-6-(1H-imidazol-1-yl)-4-methylpicolinamide (37 mg, 0.113 mmol, 27% yield) as a white solid. ¹H NMR (400 MHz, DMSO-d₆) δ 0.92-8.85 (m, 1H), 8.61 (d, J=9.6 Hz, 1H), 8.22-8.16 (m, 1H), 7.95-7.90 (m, 1H), 7.88-7.83 (m, 1H), 7.18-7.13 (m, 1H), 4.62-4.41 (m, 1H), 3.97-3.90 (m, 1H), 3.89-3.82 (m, 1H), 3.76-3.66 (m, 1H), 3.64-3.53 (m, 1H), 2.49 (s, 3H), 2.25 (s, 2H). UPLC-MS (Basic 4 min): rt=1.26 min, m/z (ES+) 323.1 [M+H]⁺

### 6-(1H-Imidazol-1-yl)-4-methyl-N-(1-((tetrahydrofuran-3-yl)sulfonyl)piperidin-4-yl)picolinamide (72A)

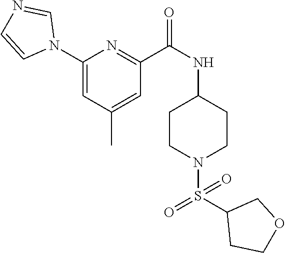 [SMILES: Cc1cc(C(=O)NC2CCN(S(=O)(=O)C3CCOC3)CC2)nc(-n2ccnc2)c1]

Oxolane-3-sulfonyl chloride (30 mg, 0.174 mmol) was added to a solution of 6-(1H-imidazol-1-yl)-4-methyl-N-(piperidin-4-yl)picolinamide hydrochloride 6 (52 mg, 0.145 mmol) and N,N-diisopropylethylamine (0.10 mL, 0.581 mmol) in DCM (0.5 mL) and the cloudy yellow suspension was stirred for 3 h. Sat. Aq. NaHCO₃ solution (2 mL) was added and the mixture was extracted with DCM (2×3 mL). The resulting combined organic layer was dried (Na₂SO₄) and concentrated under reduced pressure to give crude product. This was purified by column chromatography over silica eluting with a gradient of MeOH (1% to 8%; v/v) in DCM followed by reverse phase column chromatography over C18 eluting with a gradient of MeCN (0.1% NH₃) (5% to 55%; v/v) in water (0.1% NH₃) to afford the desired product 6-(1H-imidazol-1-yl)-4-methyl-N-(1-((tetrahydrofuran-3-yl)sulfonyl)piperidin-4-yl)picolinamide (31 mg, 0.0718 mmol, 49% yield) as a white solid. ¹H NMR (400 MHz, DMSO-d₆) δ 8.94-8.89 (m, 1H), 8.67 (d, J=8.7 Hz, 1H), 8.23 (t, J=1.4 Hz, 1H), 7.91-7.86 (m, 1H), 7.84-7.79 (m, 1H), 7.17-7.12 (m, 1H), 4.12-3.92 (m, 3H), 3.84 (ddd, J=15.8, 8.5, 5.4 Hz, 2H), 3.76-3.61 (m, 3H), 3.10-2.92 (m, 2H), 2.48 (s, 3H), 2.30-2.18 (m, 1H), 2.15-2.03 (m, 1H), 1.87-1.68 (m, 4H). UPLC-MS (Basic 4 min): rt=1.26 min, m/z (ES+) 420.2 [M+H]⁺

### 2-(1H-Imidazol-1-yl)-N-(1-(methylsulfonyl)piperidin-4-yl)pyrimidine-4-carboxamide (73A)

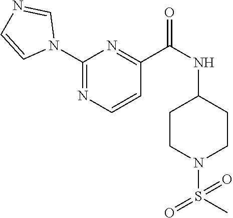 [SMILES: CS(=O)(=O)N1CCC(NC(=O)c2ccnc(-n3ccnc3)n2)CC1]

Prepared from 2-(1H-imidazol-1-yl)pyrimidine-4-carboxylic acid 9 and 1-(methylsulfonyl)-4-piperidinamine according to General Method B to afford the desired product 2-(1H-imidazol-1-yl)-N-(1-(methylsulfonyl)piperidin-4-yl)pyrimidine-4-carboxamide (45 mg, 0.128 mmol, 49% yield) as an off-white solid. ¹H NMR (400 MHz, DMSO-d₆) δ 9.07 (d, J=4.9 Hz, 1H), 9.02-8.95 (m, 2H), 8.23 (t, J=1.4 Hz, 1H), 7.92 (d, J=5.0 Hz, 1H), 7.19 (t, J=1.2 Hz, 1H), 4.02 (dq, J=15.9, 6.0 Hz, 1H), 3.63 (d, J=11.8 Hz, 2H), 2.90 (s, 3H), 2.85 (dd, J=12.2, 2.8 Hz, 2H), 1.95-1.86 (m, 2H), 1.80 (qd, J=12.2, 4.2 Hz, 2H). UPLC-MS (Basic 6 min): rt=1.55 min, m/z (ES+) 351.1 [M+H]⁺

### 6-Bromo-N-(1-(methylsulfonyl)piperidin-4-yl)-4-(trifluoromethyl)picolinamide

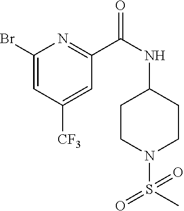 [SMILES: CS(=O)(=O)N1CCC(NC(=O)c2cc(C(F)(F)F)cc(Br)n2)CC1]

Prepared from 6-bromo-4-(trifluoromethyl)picolinic acid and 1-(methylsulfonyl)-4-piperidinamine according to General Method B to afford the desired product 6-bromo-N-(1-(methylsulfonyl)piperidin-4-yl)-4-(trifluoromethyl)picolinamide (181 mg, 0.421 mmol, 76% yield) as a white solid. ¹H NMR (400 MHz, DMSO-d₆) δ 8.85 (d, J=8.4 Hz, 1H), 8.38 (dt, J=1.5, 0.7 Hz, 1H), 8.22 (dd, J=1.5, 0.7 Hz, 1H), 3.97 (s, 1H), 3.59 (d, J=12.0 Hz, 2H), 2.89 (s, 3H), 2.84 (dd, J=12.1, 2.9 Hz, 2H), 1.95-1.67 (m, 4H). UPLC-MS (Basic 4 min): rt=1.65 min, m/z (ES+) 430.0, 432.0 [M−H]⁺

### (R)—N-(1,1-Dioxidotetrahydrothiophen-3-yl)-2-(1H-imidazol-1-yl)pyrimidine-4-carboxamide (74A)

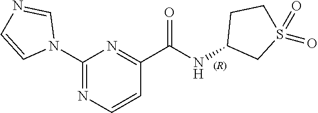

Prepared from 2-(1H-imidazol-1-yl)pyrimidine-4-carboxylic acid 9 and (3R)-1,1-dioxothiolan-3-amine hydrochloride according to General Method B to afford the desired product (R)—N-(1,1-dioxidotetrahydrothiophen-3-yl)-2-(1H-imidazol-1-yl)pyrimidine-4-carboxamide (29 mg, 0.094 mmol, 45% yield) as an off-white solid. ¹H NMR (400 MHz, DMSO-d₆) δ 9.28 (d, J=8.3 Hz, 1H), 9.10 (d, J=4.9 Hz, 1H), 8.96-8.91 (m, 1H), 8.23-8.18 (m, 1H), 7.93 (d, J=4.9 Hz, 1H), 7.22-7.17 (m, 1H), 4.82 (h, J=8.6 Hz, 1H), 3.54 (dd, J=13.2, 8.1 Hz, 1H), 3.43 (ddd, J=11.8, 7.6, 3.4 Hz, 1H), 3.30-3.20 (m, 2H), 2.46 (s, 1H), 2.41-2.27 (m, 1H). UPLC-MS (Basic 6 min): rt=1.23 min, m/z (ES+) 308.0 [M+H]⁺

### 2-(1H-Imidazol-1-yl)-N-(1-(methylsulfonyl)piperidin-4-yl)-6-(trifluoromethyl)pyrimidine-4-carboxamide (75A)

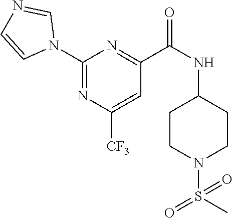 [SMILES: CS(=O)(=O)N1CCC(NC(=O)c2cc(C(F)(F)F)nc(-n3ccnc3)n2)CC1]

Prepared from 1H-imidazole 2-(1H-imidazol-1-yl)-6-(trifluoromethyl)pyrimidine-4-carboxylate 10 and 1-(methylsulfonyl)-4-piperidinamine according to General Method B to afford the desired product 2-(1H-imidazol-1-yl)-N-(1-(methylsulfonyl)piperidin-4-yl)-6-(trifluoromethyl)pyrimidine-4-carboxamide (56 mg, 0.128 mmol, 35% yield) as a white solid. ¹H NMR (400 MHz, DMSO-d₆) δ 9.14 (d, J=8.6 Hz, 1H), 9.04 (s, 1H), 8.25-8.18 (m, 2H), 7.23 (s, 1H), 4.12-3.98 (m, 1H), 3.65 (d, J=11.6 Hz, 2H), 2.93-2.83 (m, 5H), 1.96-1.88 (m, 2H), 1.88-1.73 (m, 2H). UPLC-MS (Basic 4 min): rt=1.45 min, m/z (ES+) 419.1 [M−H]⁺

### 6-Chloro-3-methyl-N-(1-(methylsulfonyl)piperidin-4-yl)picolinamide

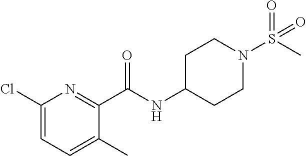 [SMILES: Cc1ccc(Cl)nc1C(=O)NC1CCN(S(C)(=O)=O)CC1]

Prepared from 6-chloro-3-methylpicolinic acid and 1-(methylsulfonyl)-4-piperidinamine according to General Method B to afford the desired product 6-chloro-3-methyl-N-(1-(methylsulfonyl)piperidin-4-yl)picolinamide (97 mg, 0.292 mmol, 62% yield) as an off-white solid. ¹H NMR (400 MHz, DMSO-d₆) δ 8.54 (d, J=8.1 Hz, 1H), 7.81 (dd, J=8.1, 0.7 Hz, 1H), 7.55 (d, J=8.1 Hz, 1H), 3.96-3.82 (m, 1H), 3.59-3.50 (m, 2H), 2.91-2.82 (m, 5H), 2.41 (s, 3H), 1.93-1.84 (m, 2H), 1.69-1.54 (m, 2H). UPLC-MS (Basic 2 min): rt=0.92 min, m/z (ES+) 332.2 [M−H]⁺

### (R)-2-(1H-Imidazol-1-yl)-N-(tetrahydro-2H-pyran-3-yl)-6-(trifluoromethyl)pyrimidine-4-carboxamide (76A)

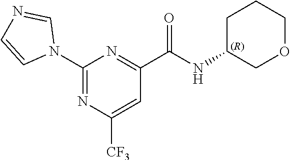

Prepared from 1H-imidazole 2-(1H-imidazol-1-yl)-6-(trifluoromethyl)pyrimidine-4-carboxylate 10 and (R)-tetrahydro-2H-pyran-3-amine hydrochloride according to General Method B to afford the desired product (R)-2-(1H-imidazol-1-yl)-N-(tetrahydro-2H-pyran-3-yl)-6-(trifluoromethyl)pyrimidine-4-carboxamide (34 mg, 0.099 mmol, 68% yield) as a white solid. ¹H NMR (400 MHz, DMSO-d₆) δ 9.04-8.95 (m, 2H), 8.24-8.17 (m, 2H), 7.22 (s, 1H), 4.09-3.95 (m, 1H), 3.86-3.77 (m, 2H), 3.37-3.26 (m, 2H), 1.99-1.91 (m, 1H), 1.87-1.55 (m, 3H). UPLC-MS (Basic 4 min): rt=1.46 min, m/z (ES+) 342.2 [M−H]⁺

### (R)—N-(1,1-Dioxidotetrahydrothiophen-3-yl)-2-(1H-imidazol-1-yl)-6-(trifluoromethyl)pyrimidine-4-carboxamide (77A)

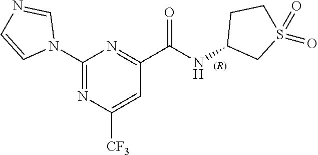

Prepared from 1H-imidazole 2-(1H-imidazol-1-yl)-6-(trifluoromethyl)pyrimidine-4-carboxylate 10 and (3R)-1,1-dioxothiolan-3-amine hydrochloride according to General Method B to afford the desired product (R)—N-(1,1-dioxidotetrahydrothiophen-3-yl)-2-(1H-imidazol-1-yl)-6-(trifluoromethyl)pyrimidine-4-carboxamide (12 mg, 0.031 mmol, 21% yield) as a white solid. ¹H NMR (400 MHz, DMSO-d₆) δ 9.41 (d, J=8.2 Hz, 1H), 8.98 (s, 1H), 8.24-8.17 (m, 2H), 7.24 (s, 1H), 4.91-4.76 (m, 1H), 3.62-3.52 (m, 1H), 3.51-3.38 (m, 1H), 3.34-3.22 (m, 2H), 2.50-2.42 (m, 1H), 2.41-2.26 (m, 1H). UPLC-MS (Basic 4 min): rt=1.30 min, m z (ES+) 376.2 [M+H]⁺

### (R)-6-Bromo-N-(1,1-dioxidotetrahydrothiophen-3-yl)-4-(trifluoromethyl)picolinamide

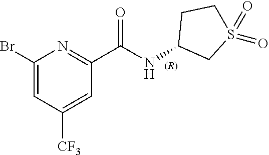

Prepared from 6-bromo-4-(trifluoromethyl)picolinic acid and (3R)-1,1-dioxothiolan-3-amine hydrochloride according to General Method B to afford the desired product (R)-6-bromo-N-(1,1-dioxidotetrahydrothiophen-3-yl)-4-(trifluoromethyl)picolinamide (110 mg, 0.278 mmol, 58% yield) as a white solid. ¹H NMR (400 MHz, DMSO-d₆) δ 9.28 (d, J=8.1 Hz, 1H), 8.44-8.39 (m, 1H), 8.26-8.21 (m, 1H), 4.84-4.69 (m, 1H), 3.51-3.34 (m, 2H), 3.33-3.17 (m, 2H), 2.48-2.23 (m, 2H). UPLC-MS (Basic 2 min): rt=0.99 min, m/z (ES+) 385.0, 387.0 [M+H]⁺

### (R)—N-(1,1-Dioxidotetrahydrothiophen-3-yl)-6-(1H-imidazol-1-yl)-4-(trifluoromethyl)picolinamide (78A)

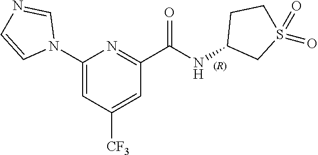

Prepared from (R)-6-bromo-N-(1,1-dioxidotetrahydrothiophen-3-yl)-4-(trifluoromethyl)picolinamide according to General Method H to afford the desired product (R)—N-(1,1-dioxidotetrahydrothiophen-3-yl)-6-(1H-imidazol-1-yl)-4-(trifluoromethyl)picolinamide (15 mg, 0.0401 mmol, 14% yield) as a white solid. ¹H NMR (400 MHz, DMSO-d₆) δ 9.16 (d, J=8.4 Hz, 1H), 9.06 (t, J=1.1 Hz, 1H), 8.52-8.47 (m, 1H), 8.41 (t, J=1.4 Hz, 1H), 8.16-8.11 (m, 1H), 7.24-7.18 (m, 1H), 4.91-4.76 (m, 1H), 3.53 (dd, J=13.2, 8.0 Hz, 1H), 3.49-3.37 (m, 1H), 3.30-3.21 (m, 2H), 2.48-2.27 (m, 2H). UPLC-MS (Basic 4 min): rt=1.25 min, m z (ES+) 375.1 [M+H]⁺

### 6-(1H-Imidazol-1-yl)-3-methyl-N-(1-(methylsulfonyl)piperidin-4-yl)picolinamide (79A)

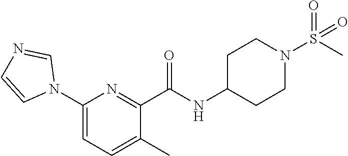 [SMILES: Cc1ccc(-n2ccnc2)nc1C(=O)NC1CCN(S(C)(=O)=O)CC1]

Prepared from 6-chloro-3-methyl-N-(1-(methylsulfonyl)piperidin-4-yl)picolinamide according to General Method H to afford the desired product 6-(1H-imidazol-1-yl)-3-methyl-N-(1-(methylsulfonyl)piperidin-4-yl)picolinamide (26 mg, 0.0681 mmol, 24% yield) as an off-white solid. ¹H NMR (400 MHz, DMSO-d₆) δ 8.71 (s, 1H), 8.54 (d, J=8.4 Hz, 1H), 8.10-8.05 (m, 1H), 7.91 (dd, J=8.3, 0.7 Hz, 1H), 7.83 (d, J=8.4 Hz, 1H), 7.10 (s, 1H), 3.97-3.85 (m, 1H), 3.59-3.48 (m, 2H), 2.88-2.77 (m, 5H), 2.50 (s, 3H), 1.90-1.81 (m, 2H), 1.69 (qd, J=12.1, 4.2 Hz, 2H). UPLC-MS (Basic 2 min): rt=1.12 min, m/z (ES+) 364.2 [M+H]⁺

### (R)-2-(1H-Imidazol-1-yl)-N-(tetrahydro-2H-pyran-3-yl)pyrimidine-4-carboxamide (80A)

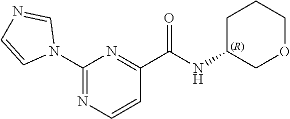

Prepared from 2-(1H-imidazol-1-yl)pyrimidine-4-carboxylic acid 9 and (R)-tetrahydro-2H-pyran-3-amine hydrochloride according to General Method B to afford the desired product (R)-2-(1H-imidazol-1-yl)-N-(tetrahydro-2H-pyran-3-yl)pyrimidine-4-carboxamide (44 mg, 0.161 mmol, 76% yield) as a white solid.

¹H NMR (400 MHz, DMSO-d₆) δ 9.07 (d, J=5.0 Hz, 1H), 8.95 (t, J=1.1 Hz, 1H), 8.83 (d, J=8.6 Hz, 1H), 8.22 (t, J=1.4 Hz, 1H), 7.91 (d, J=5.0 Hz, 1H), 7.19 (dd, J=1.5, 0.9 Hz, 1H), 3.99 (ddq, J=13.9, 9.4, 4.8 Hz, 1H), 3.80 (ddd, J=10.6, 4.7, 1.9 Hz, 2H), 3.32-3.26 (m, 2H), 1.93 (dd, J=10.6, 5.1 Hz, 1H), 1.86-1.54 (m, 3H). UPLC-MS (Basic 6 min): rt=1.56 min, m/z (ES+) 274.1 [M+H]⁺

### (R)-6-(1H-Imidazol-1-yl)-3-methyl-N-(tetrahydro-2H-pyran-3-yl)picolinamide (81A)

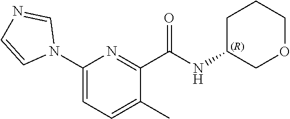

Prepared from (R)-6-chloro-3-methyl-N-(tetrahydro-2H-pyran-3-yl)picolinamide according to General Method H to afford the desired product (R)-6-(1H-imidazol-1-yl)-3-methyl-N-(tetrahydro-2H-pyran-3-yl)picolinamide (31 mg, 0.103 mmol, 40% yield) as a white solid. ¹H NMR (400 MHz, ACETONITRILE-d₃) δ 8.37 (t, J=1.1 Hz, 1H), 7.85 (dq, J=8.5, 0.7 Hz, 2H), 7.78 (t, J=1.4 Hz, 1H), 7.62-7.55 (m, 1H), 7.17-7.10 (m, 1H), 4.00 (ddq, J=12.2, 8.2, 3.9 Hz, 1H), 3.84 (ddd, J=11.0, 3.8, 1.4 Hz, 1H), 3.73 (dt, J=11.4, 4.3 Hz, 1H), 3.51 (ddd, J=11.3, 8.5, 3.0 Hz, 1H), 3.40 (dd, J=11.0, 7.7 Hz, 1H), 2.64 (t, J=0.5 Hz, 3H), 2.01-1.96 (m, 1H), 1.82-1.58 (m, 3H). UPLC-MS (Basic 6 min): rt=1.72 min m/z (ES+) 287.2 [M+H]⁺

### 6-(1H-Imidazol-1-yl)-N-(1-(methylsulfonyl)piperidin-4-yl)-4-(trifluoromethyl)picolinamide (82A)

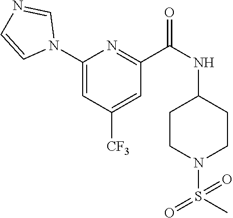 [SMILES: CS(=O)(=O)N1CCC(NC(=O)c2cc(C(F)(F)F)cc(-n3ccnc3)n2)CC1]

Prepared from 6-bromo-N-(1-(methylsulfonyl)piperidin-4-yl)-4-(trifluoromethyl)picolinamide according to General Method H to afford the desired product 6-(1H-imidazol-1-yl)-N-(1-(methylsulfonyl)piperidin-4-yl)-4-(trifluoromethyl)picolinamide (60 mg, 0.143 mmol, 41% yield) as an off-white solid. ¹H NMR (400 MHz, DMSO-d₆) δ 9.09 (s, 1H), 8.87 (d, J=8.7 Hz, 1H), 8.46 (dd, J=1.3, 0.7 Hz, 1H), 8.43 (t, J=1.4 Hz, 1H), 8.12 (dd, J=1.3, 0.6 Hz, 1H), 7.20 (dd, J=1.6, 0.9 Hz, 1H), 4.12-3.97 (m, 1H), 3.63 (d, J=11.7 Hz, 2H), 2.90 (s, 3H), 2.85 (dd, J=12.1, 3.0 Hz, 2H), 1.98-1.68 (m, 4H). UPLC-MS (Basic 4 min): rt=1.37 min, m/z (ES+) 418.1 [M+H]⁺

### (R)-6-Chloro-3-methyl-N-(tetrahydro-2H-pyran-3-yl)picolinamide

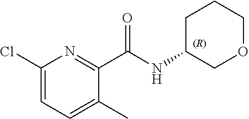

Prepared from 6-chloro-3-methylpicolinic acid and (R)-tetrahydro-2H-pyran-3-amine hydrochloride according to General Method B to afford the desired product (R)-6-chloro-3-methyl-N-(tetrahydro-2H-pyran-3-yl)picolinamide (126 mg, 0.494 mmol, 99% yield) as an off-white solid. ¹H NMR (400 MHz, DMSO-d₆) δ 8.41 (d, J=8.2 Hz, 1H), 7.81 (dq, J=8.1, 0.7 Hz, 1H), 7.55 (d, J=8.1 Hz, 1H), 3.94-3.81 (m, 1H), 3.80-3.66 (m, 2H), 3.38-3.34 (m, 1H), 3.25 (dd, J=10.8, 8.7 Hz, 1H), 2.42 (d, J=0.6 Hz, 3H), 1.93-1.84 (m, 1H), 1.73-1.49 (m, 3H). UPLC-MS (Basic 2 min): rt=0.97 min m/z (ES+) 255.1 [M−H]⁺

### (R)-6-(1H-Imidazol-1-yl)-4-methyl-N-(1-(oxetan-3-yl)piperidin-3-yl)picolinamide (83A)

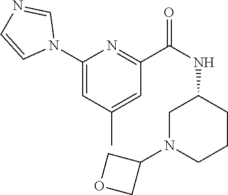 [SMILES: Cc1cc(C(=O)N[C@@H]2CCCN(C3COC3)C2)nc(-n2ccnc2)c1]

Prepared from lithium 6-(1H-imidazol-1-yl)-4-methylpicolinate 5 and (3R)-1-(oxetan-3-yl)piperidin-3-amine according to General Method B to afford the desired product (R)-6-(1H-imidazol-1-yl)-4-methyl-N-(1-(oxetan-3-yl)piperidin-3-yl)picolinamide (9.0 mg, 0.028 mmol, 15% yield) as a white solid. ¹H NMR (400 MHz, DMSO-d₆) δ 8.87 (t, J=1.1 Hz, 1H), 8.58 (d, J=8.7 Hz, 1H), 8.19 (t, J=1.4 Hz, 1H), 7.89 (dd, J=1.4, 0.8 Hz, 1H), 7.82 (dd, J=1.3, 0.7 Hz, 1H), 7.14 (dd, J=1.5, 0.9 Hz, 1H), 4.53 (td, J=6.5, 2.0 Hz, 2H), 4.43 (td, J=6.2, 2.7 Hz, 2H), 4.01 (dd, J=11.4, 6.8 Hz, 1H), 3.44 (p, J=6.4 Hz, 1H), 2.68-2.60 (m, 1H), 2.56-2.52 (m, 1H), 2.49-2.47 (m, 3H), 2.00 (t, J=9.9 Hz, 1H), 1.88 (s, 1H), 1.82-1.67 (m, 2H), 1.64-1.47 (m, 2H). UPLC-MS (Basic 6 min): rt=1.83 min, m/z (ES+) 342.2 [M+H]⁺

### (S)—N-(1,1-Dioxidotetrahydro-2H-thiopyran-3-yl)-6-(1H-imidazol-1-yl)-4-methylpicolinamide (84A)

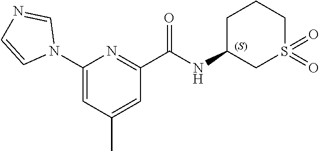

Prepared from lithium 6-(1H-imidazol-1-yl)-4-methylpicolinate 5 and (3S)-1,1-dioxothian-3-amine according to General Method B to afford the desired product (S)—N-(1,1-dioxidotetrahydro-2H-thiopyran-3-yl)-6-(1H-imidazol-1-yl)-4-methylpicolinamide (18 mg, 0.053 mmol, 45% yield) as a white solid. ¹H NMR (400 MHz, DMSO-d₆) δ 9.03 (d, J=8.7 Hz, 1H), 8.83 (t, J=1.2 Hz, 1H), 8.17 (t, J=1.4 Hz, 1H), 7.92 (dd, J=1.3, 0.8 Hz, 1H), 7.83 (dd, J=1.3, 0.7 Hz, 1H), 7.16 (dd, J=1.5, 0.9 Hz, 1H), 4.49-4.39 (m, 1H), 3.44-3.33 (m, 2H), 3.22-3.04 (m, 2H), 2.49 (s, 3H), 2.08 (s, 1H), 1.93-1.70 (m, 3H). UPLC-MS (Basic 4 min): rt=1.09 min, m/z (ES+) 335.2 [M+H]⁺

### tert-Butyl (S)-3-(6-(1H-imidazol-1-yl)-4-methylpicolinamido)pyrrolidine-1-carboxylate

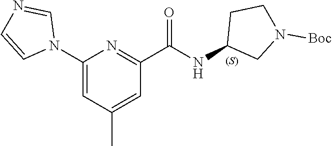

Prepared from cesium 6-(1H-imidazol-1-yl)-4-methylpicolinate 4 and tert-butyl (3S)-3-aminopyrrolidine-1-carboxylate according to General Method A to afford the desired product tert-butyl (S)-3-(6-(1H-imidazol-1-yl)-4-methylpicolinamido)pyrrolidine-1-carboxylate (33 mg, 0.089 mmol, 49% yield) as a colourless gum. ¹H NMR (400 MHz, CHLOROFORM-d) δ 8.40-8.26 (m, 1H), 7.98 (dd, J=1.3, 0.7 Hz, 1H), 7.84-7.77 (m, 1H), 7.61 (t, J=1.4 Hz, 1H), 7.36-7.31 (m, 1H), 7.24-7.20 (m, 1H), 4.70-4.61 (m, 1H), 3.81-3.68 (m, 1H), 3.60-3.44 (m, 2H), 3.41-3.25 (m, 1H), 2.55-2.50 (m, 3H), 2.36-2.23 (m, 1H), 1.99 (s, 1H), 1.19 (s, 9H). UPLC-MS (Acidic 2 min): rt=0.99 min, m/z (ES+) 372.3 [M+H]⁺.

### tert-Butyl (S)-3-(6-chloro-4-methylpicolinamido)piperidine-1-carboxylate

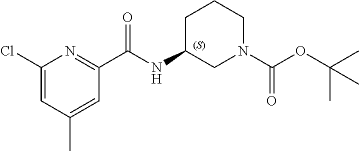

6-Chloro-4-methylpyridine-2-carboxylic acid (200 mg, 1.17 mmol), 0-(7-azabenzotriazol-1-yl)-N,N,N′,N′-tetramethyluronium hexafluorophosphate (665 mg, 1.75 mmol) and N,N-diisopropylethylamine (0.41 mL, 2.33 mmol) were dissolved in DMF (2 mL) and stirred for 30 minutes at 25° C. tert-Butyl (3S)-3-aminopiperidine-1-carboxylate (327 mg, 1.63 mmol) was added and the reaction stirred for 3 h. This was diluted withy ethyl acetate (20 ml) and washed with water (2×20 ml) and brine (20 ml). The resulting organic layer was dried (Na₂SO₄) and concentrated under reduced pressure to give crude product. This was purified by column chromatography over silica eluting with a gradient of MeOH (0% to 10%; v/v) in DCM to afford the desired product tert-butyl (S)-3-(6-chloro-4-methylpicolinamido)piperidine-1-carboxylate (280 mg, 0.752 mmol, 64% yield) as a yellow oil. ¹H NMR (400 MHz, DMSO-d₆) δ 8.35 (s, 1H), 7.87 (s, 1H), 7.58 (s, 1H), 3.88-3.52 (m, 3H), 3.19-2.86 (m, 2H), 2.41 (s, 3H), 1.86-1.78 (m, 1H), 1.77-1.60 (m, 2H), 1.48-1.29 (m, 10H). UPLC-MS (Acidic 4 min): rt=2.08 min, m/z (ES+) 281.2 [M-tBuO+H]⁺

### (S)-6-(1H-Imidazol-1-yl)-4-methyl-N-(1-methyl-2-oxopiperidin-3-yl)picolinamide (85A)

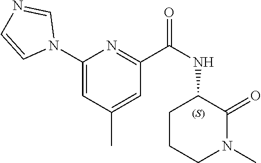

Prepared from lithium 6-(1H-imidazol-1-yl)-4-methylpicolinate 5 and (S)-3-amino-1-methylpiperidin-2-one hydrochloride according to General Method B to afford the desired product (S)-6-(1H-imidazol-1-yl)-4-methyl-N-(1-methyl-2-oxopiperidin-3-yl)picolinamide (22 mg, 0.069 mmol, 36% yield) as a white solid. ¹H NMR (400 MHz, DMSO-d₆) δ 9.08 (d, J=8.6 Hz, 1H), 8.88 (t, J=1.1 Hz, 1H), 8.22 (t, J=1.4 Hz, 1H), 7.90 (t, J=1.1 Hz, 1H), 7.88-7.80 (m, 1H), 7.15 (dd, J=1.5, 0.9 Hz, 1H), 4.51-4.40 (m, 1H), 3.36-3.31 (m, 2H), 2.87 (s, 3H), 2.49 (s, 3H), 2.10-1.85 (m, 4H). UPLC-MS (Basic 4 min): rt=1.11 min, m/z (ES+) 314.1 [M+H]⁺.

### (R)—N-(1,1-Dioxidotetrahydrothiophen-3-yl)-6-(1H-imidazol-1-yl)-4-methylpicolinamide (86A)

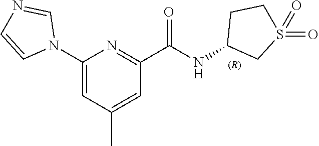

Prepared from lithium 6-(1H-imidazol-1-yl)-4-methylpicolinate 5 and (3R)-1,1-dioxothiolan-3-amine hydrochloride according to General Method B to afford the desired product (R)—N-(1,1-dioxidotetrahydrothiophen-3-yl)-6-(1H-imidazol-1-yl)-4-methylpicolinamide (7 mg, 0.021 mmol, 18% yield) as a white solid. ¹H NMR (400 MHz, DMSO-d₆) δ 8.99 (d, J=8.4 Hz, 1H), 8.89 (dd, J=1.2, 1.2 Hz, 1H), 8.21 (dd, J=1.4, 1.4 Hz, 1H), 7.94-7.89 (m, 1H), 7.83 (dd, J=1.3, 0.7 Hz, 1H), 7.16 (dd, J=1.2, 1.2 Hz, 1H), 5.00-4.64 (m, 1H), 3.63-3.45 (m, 1H), 3.45-3.38 (m, 1H), 3.31-3.28 (m, 1H), 3.27-3.18 (m, 1H), 2.49 (s, 3H), 2.43 (ddt, J=10.3, 7.0, 3.3 Hz, 1H), 2.33 (dtd, J=13.0, 10.3, 8.0 Hz, 1H). UPLC-MS (Basic 4 min): rt=1.05 min, m/z (ES+) 321.1 [M+H]⁺.

### 6-(1H-Imidazol-1-yl)-4-methyl-N-(1-(oxetan-3-ylsulfonyl)piperidin-4-yl)picolinamide (87A)

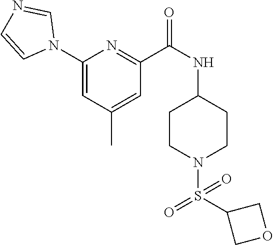 [SMILES: Cc1cc(C(=O)NC2CCN(S(=O)(=O)C3COC3)CC2)nc(-n2ccnc2)c1]

Oxetane-3-sulfonyl chloride (27 mg, 0.174 mmol) was added to a solution of 6-(1H-imidazol-1-yl)-4-methyl-N-(piperidin-4-yl)picolinamide hydrochloride 6 (52 mg, 0.145 mmol) and N,N-diisopropylethylamine (0.10 mL, 0.574 mmol) in DCM (0.50 mL) and the cloudy yellow suspension was stirred for 3 h. Aq. sat. NaHCO₃ solution (2 mL) was added and the mixture was extracted with DCM (2×3 mL). The resulting combined organic layer was dried (Na₂SO₄) and concentrated under reduced pressure to give crude product. This was purified by column chromatography over silica eluting with a gradient of MeOH (1% to 8%; v/v) in DCM to afford a white solid. This was triturated with MTBE to afford the desired product 6-(1H-imidazol-1-yl)-4-methyl-N-(1-(oxetan-3-ylsulfonyl)piperidin-4-yl)picolinamide (28 mg, 0.0674 mmol, 46% yield) as a white solid. ¹H NMR (400 MHz, DMSO-d₆) δ 8.93 (s, 1H), 8.67 (d, J=8.7 Hz, 1H), 8.23 (s, 1H), 7.91-7.86 (m, 1H), 7.84-7.79 (m, 1H), 7.15 (s, 1H), 4.88-4.69 (m, 5H), 4.09-3.93 (m, 1H), 3.75-3.54 (m, 2H), 2.91 (td, J=12.4, 2.7 Hz, 2H), 2.47 (s, 3H), 1.88-1.67 (m, 4H). UPLC-MS (Basic 4 min): rt=0.85 min, m/z (ES+) 406.2 [M+H]⁺.

### Example 5: NAD⁺ Cydase Activity Assay with Human or Mouse Recombinant CD38 Protein

The following assay was used to determine the ability of compounds of the invention to inhibit the hydrolysis of NAD⁺ by the human or mouse enzyme, CD38.

### Final Assay Conditions for Assay Protocol

Reaction buffer: 40 mM HEPES/NaOH pH 7.5, 250 mM sucrose, 0.01% Tween

Final concentrations: 0.6 nM mouse CD38 (4947-AC, Bio-Techne GmbH) 1.9 nM human CD38 (AVI2404, Bio-Techne GmbH)

Substrate: 15.8 μM Nicotinamide 1,N6-ethenoadenine dinucleotide (E-NAD)

Read-out: Fluorescence intensity (extinction 300 nm/emission 410 nm)

### Assay Protocol

Required reaction buffer, required CD38 concentration in reaction buffer and required e-NAD concentration in reaction buffer were prepared. Thereafter, 8 μL of CD38 in reaction buffer were added to High Control and compound concentration wells in 384 well plate (Corning #4514, 384 well, nonbinding surface, low volume, round bottom) and 8 μL of reaction buffer to Low Control. Compound dilutions were transferred via pintool technology that transfers 42.5 nL compound dilutions (12-point dose-response with factor three dilutions in 100% DMSO) well-to-well. Plate was subsequently shaken for 15 s at 1450 rpm and sealed for incubation for 30 min at room temperature. Thereafter, 2 μL of e-NAD in reaction buffer were added with Integra Voyager. After shaking for 15 s at 1450 rpm, plate was sealed, and assay signal was measured continuously with PHERAstar® fluorescence intensity module (extinction 300 nm/emission 410 nm).

### Data Analysis

Dose-response curves were constructed using twelve-point factor three dilutions in triplicates per plate. High control (full enzyme activity) was set to assay signal of CD38 and e-NAD in presence of vehicle (DMSO) and Low control (full enzyme inhibition) was set to assay signal of e-NAD and vehicle (DMSO) in absence of CD38. For data analysis, fluorescent signal of traces corresponding to each compound concentration and High and Low control was monitored over time. Each run, High Control (full enzyme activity) was fitted to Linear equation via GraphPad Prism to determine CD38 activity from the slope and determine the time interval of linear phase of reaction to account for minor fluctuations. Thereafter, slopes of all compound concentrations were determined via fitting to same linear equation (GraphPad Prism) over the same time interval as determined for High control. Percentage inhibition was calculated via normalization to High and Low Control via the equation: %−inhibition=100*(1−((Sample−Low control)/(High control−Low control))) Half maximal inhibitory concentration (IC50) values are yielded from the inflection point of the corresponding curve by fitting of resulting percentage inhibition values to standard equation (4 Parameter Logistic Model or Sigmoidal Dose-Response Model) via Excel Fit. Unless otherwise stated, fitting of curves was fixed to 100%-inhibition and unlocked for 0%-inhibition.

Compounds of the invention are potent inhibitors of CD38 as demonstrated by the results shown in the Table 1. For Human CD38, compounds having an IC₅₀ (nM) of less than 30.0 nM as ++++, 30.0 nM to 100 nM as +++, 100 nM to 1000 nM as ++, and greater than 1000 nM as +. For Mouse CD38, compounds having an IC₅₀ (nM) of less than 1.0 nM++++, 1.0 nM to 10.0 nM as +++, 10.0 nM to 100 nM as ++, and greater than 100.0 nM as +.

TABLE 1
CD38 IC₅₀ Values

| Compound Number | Human CD38 IC50 | Mouse CD38 IC50 |
|---|---|---|
| 1A | +++ | +++ |
| 2A | ++++ | +++ |
| 3A | ++++ | +++ |
| 4A | ++++ | +++ |
| 5A | ++++ | +++ |
| 6A | ++++ | +++ |
| 7A | +++ | +++ |
| 8A | ++++ | +++ |
| 9A | ++++ | +++ |
| 10A | +++ | +++ |
| 11A | + | +++ |
| 12A | +++ | +++ |
| 13A | +++ | +++ |
| 14A | ++ | +++ |
| 15A | +++ | +++ |
| 16A | +++ | +++ |
| 18A | ++++ | +++ |
| 19A | +++ | +++ |
| 20A | +++ | +++ |
| 21A | +++ | +++ |
| 22A | ++++ | +++ |
| 23A | +++ | +++ |
| 24A | ++ | +++ |
| 25A | +++ | +++ |
| 26A | +++ | +++ |
| 27A | ++ | ++ |
| 28A | ++ | +++ |
| 29A | +++ | +++ |
| 30A | +++ | +++ |
| 31A | +++ | +++ |
| 32A | +++ | +++ |
| 34A | ++++ | +++ |
| 35A | ++++ | +++ |
| 36A | ++ | +++ |
| 38A | ++ | +++ |
| 41A | + | + |
| 42A | +++ | +++ |
| 43A | ++++ | +++ |
| 44A | ++++ | ++++ |
| 45A | ++ | +++ |
| 46A | ++ | +++ |
| 47A | ++ | +++ |
| 48A | + | ++ |
| 49A | +++ | +++ |
| 50A | ++ | +++ |
| 51A | + | + |
| 52A | + | + |
| 53A | ++ | +++ |
| 54A | +++ | +++ |
| 55A | ++ | +++ |
| 56A | + | + |
| 57A | +++ | +++ |
| 58A | + | ++ |
| 59A | + | ++ |
| 60A | ++ | +++ |
| 61A | ++++ | ++++ |
| 62A | ++++ | +++ |
| 63A | + | ++ |
| 64A | +++ | +++ |
| 65A | ++ | +++ |
| 66A | +++ | ++++ |
| 67A | ++ | +++ |
| 68A | ++++ | ++++ |
| 69A | ++++ | ++++ |
| 70A | +++ | +++ |
| 71A | +++ | ++++ |
| 72A | ++++ | ++++ |
| 73A | ++++ | +++ |
| 74A | ++++ | +++ |
| 75A | +++ | +++ |
| 76A | +++ | +++ |
| 77A | +++ | +++ |
| 78A | ++++ | +++ |
| 79A | ++ | ++ |
| 80A | ++++ | +++ |
| 81A | ++ | ++ |
| 82A | ++++ | +++ |
| 83A | + | ++ |
| 84A | ++ | ++ |
| 85A | ++ | ++ |
| 86A | ++++ | +++ |
| 87A | ++++ | +++ |

1-141. (canceled)

142. A compound of Formula (I): 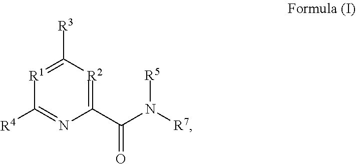
or a pharmaceutically acceptable salt thereof, wherein:
R¹ is selected from N and CR¹¹;
R² is selected from N and CR¹²;
R³ is selected from hydrogen, halogen, —CN, —OR⁶, —SO₂R¹⁶, C₁-C₆ alkyl, C₃₋₁₂ carbocycle, and 3- to 12-membered heterocycle, wherein the C₁-C₆ alkyl, C₃₋₁₂ carbocycle, and 3- to 12-membered heterocycle are each optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl, C₁-C₆ alkyl, C₂₋₁₀ alkenyl, C₂₋₁₀ alkynyl, C₃₋₁₂ carbocycle, and 3- to 12-membered heterocycle;
R⁴ is selected from a 5- to 6-membered heteroaryl, wherein the 5- to 6-membered heteroaryl is selected from imidazole, thiazole, oxazole, pyrimidine, pyrazine, pyridazine, oxadiazole, and thiadiazole, each of which is optionally substituted with one or more R⁹;
R⁵ is selected from hydrogen and C₁-C₆ alkyl;
R⁷ is selected from:
a saturated 4- to 8-membered heterocycle, wherein the saturated 4- to 8-membered heterocycle is substituted with at least one substituent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the saturated 4- to 8-membered heterocycle is further optionally substituted with one or more R⁸;
a C₃₋₅ cycloalkyl, and C₇₋₁₀ cycloalkyl, each of which is substituted with at least one substituent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein each is further optionally substituted with one or more R⁸;
a C₆ cycloalkyl substituted with at least one substituent selected from —S(O)₂(R²⁰), —S(O)(R²⁰), —S(O)₂(NR²⁰₂), —S(O)(NR²¹)R²⁰; and wherein the C₆ cycloalkyl is further optionally substituted with one or more R^{8A};
R⁸ is independently selected at each occurrence from halogen, —OR²⁰, —SR²⁰, —N(R²¹)₂, —NO₂, ═O, ═S, ═N(R²⁰), —CN, C₁₋₆ aminoalkyl, C₁₋₆ hydroxyalkyl, C₁₋₆ cyanoalkyl, C₁₋₆ haloalkyl, C₁₋₆ alkoxyalkyl, C₁₋₆ alkyl, C₂₋₆ alkenyl, C₂₋₆ alkynyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, —S(O)(NR²¹)N(R²¹)₂, and 4- to 8-membered heterocycle;
R^{8A} is independently selected at each occurrence from halogen, C₁₋₆ haloalkyl, —C(O)R²⁰, —C(O)OR²⁰, —C(O)N(R²¹)₂, —N(R²¹)C(O)R²⁰, —N(R²¹)C(O)N(R²¹)₂, —S(O)₂(R²⁰), —S(O)(R²⁰), —S(O)₂(NR²⁰₂), —S(O)(NR²¹)R²⁰, and —S(O)(NR²¹)N(R²¹)₂;
each R⁹ is independently selected from halogen, —OH, —CN, —NO₂, —NH₂, —NHC₁₋₁₀ alkyl, —N(C₁₋₁₀ alkyl)₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R¹¹ is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹² is selected from hydrogen, halogen, —OH, —CN, —NO₂, —NH₂, —NHC₁₋₁₀ alkyl, —N(C₁₋₁₀ alkyl)₂, —O—C₁₋₁₀ alkyl, C₁-C₆ alkyl, and C₁-C₆ haloalkyl;
R¹⁶ is selected from hydrogen; C₁₋₆ alkyl, and C₃₋₁₂ carbocycle, wherein the C₁₋₆ alkyl, and C₃₋₁₂ carbocycle are each optionally substituted with one or more substituents independently selected from halogen, —OH, —CN, —NO₂, —NH₂, —NHC₁₋₁₀ alkyl, —N(C₁₋₁₀ alkyl)₂, —O—C₁₋₁₀ alkyl, C₂₋₁₀ alkenyl, C₁-C₆ alkyl, C₁-C₆ haloalkyl, C₂₋₁₀ alkynyl, C₃₋₁₂ carbocycle, and 3- to 12-membered heterocycle;
R²⁰ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle; and
R²¹ is independently selected at each occurrence from hydrogen; and C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents independently selected from halogen, —OH, —CN, —NO₂, —NH₂, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle.

143. The compound or salt of claim 142, wherein Formula (I) is represented by Formula (I-A) 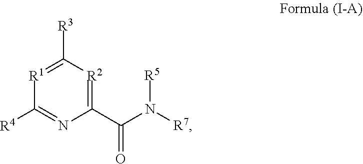
or a pharmaceutically acceptable salt thereof, wherein:
R³ is selected from halogen, —O—C₁-C₆ alkyl, and C₁-C₆ alkyl, wherein the C₁-C₆ alkyl is optionally substituted with one or more substituents independently selected from halogen, —OH, —CN, —NO₂, —NH₂, —NHC₁₋₁₀ alkyl, —N(C₁₋₁₀ alkyl)₂, —C₁₋₁₀ haloalkyl, —O—C₁₋₁₀ alkyl; and
R⁷ is selected from an optionally substituted saturated 4- to 8-membered heterocycle, which is optionally substituted with one or more R₈.

144. The compound or salt of claim 143, wherein R¹ is CH or N; and R² is CH or CR¹² wherein R¹² is selected from C₁₋₆ alkyl.

145. The compound or salt of claim 144, wherein R¹ is CH.

146. The compound or salt of claim 144, wherein R¹ is N.

147. The compound or salt of claim 144, wherein R³ is selected from hydrogen, —O—C₁-C₆ alkyl, and C₁-C₆ alkyl, wherein the C₁-C₆ alkyl is optionally substituted with one or more substituents independently selected from halogen.

148. The compound or salt of claim 147, wherein R³ is selected from hydrogen —CF₃, —CH₃, and —OCH₃.

149. The compound or salt of claim 148, wherein R⁷ is selected from a saturated 4- to 8-membered heterocycle, wherein the saturated 4- to 8-membered heterocycle is substituted with at least one substituent selected from —S(O)₂(R²⁰), —S(O)(R²⁰), —S(O)₂(NR²⁰₂), —S(O)(NR²¹)R²⁰, and —S(O)(NR²¹)N(R²¹)₂; and wherein the saturated 4- to 8-membered heterocycle is further optionally substituted with one or more R⁸.

150. The compound or salt of claim 149, wherein the saturated 4- to 8-membered_heterocycle of R⁷ is selected from 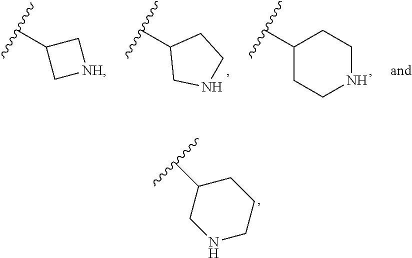 [SMILES: *C1CNC1 ; *C1CCNC1 ; *C1CCNCC1 ; *C1CCCNC1]
wherein the saturated 4- to 8-membered heterocycle of R⁷ is substituted with at least one substituent-selected from —S(O)₂(R²⁰), and —S(O)₂(NR²⁰₂).

151. The compound or salt of claim 150, wherein R⁷ is selected from

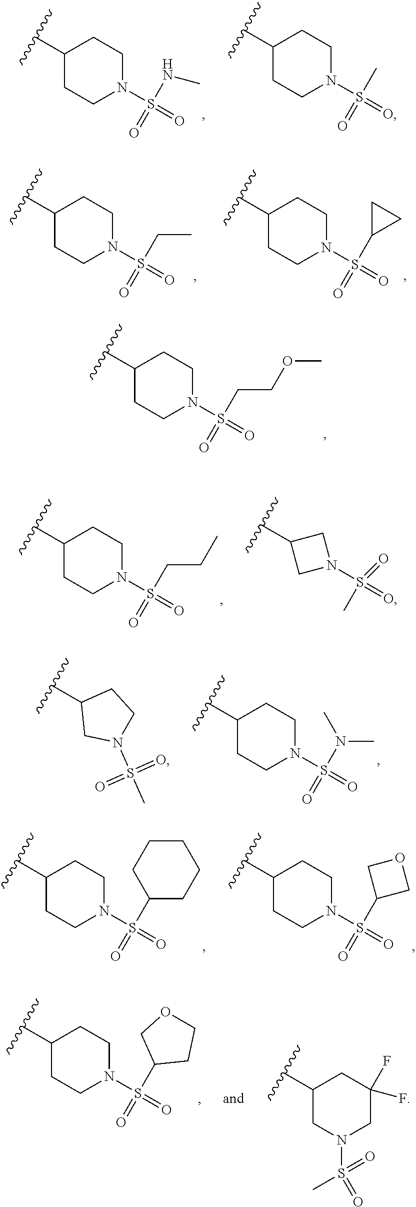

152. The compound or salt of claim 151, wherein R⁵ is hydrogen.

153. The compound or salt of claim 152, wherein R⁴ is

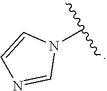 [SMILES: *n1ccnc1]

154. The compound or salt of claim 142, selected from: 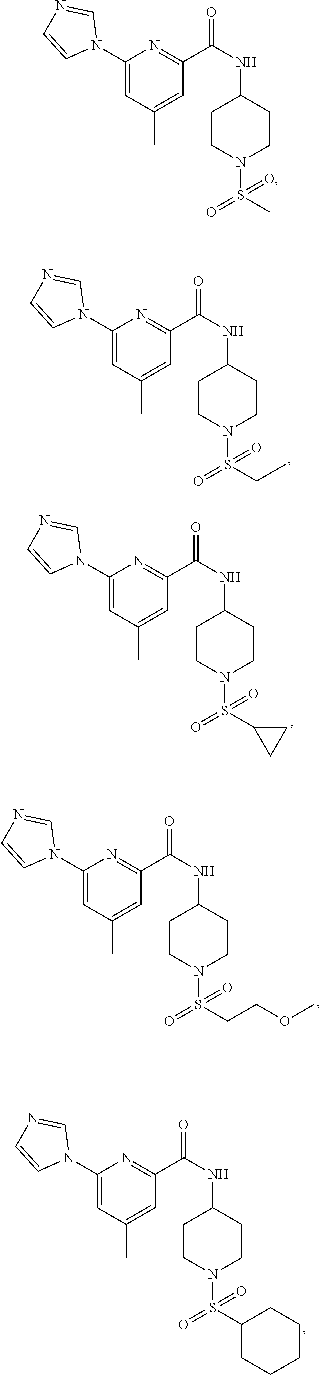 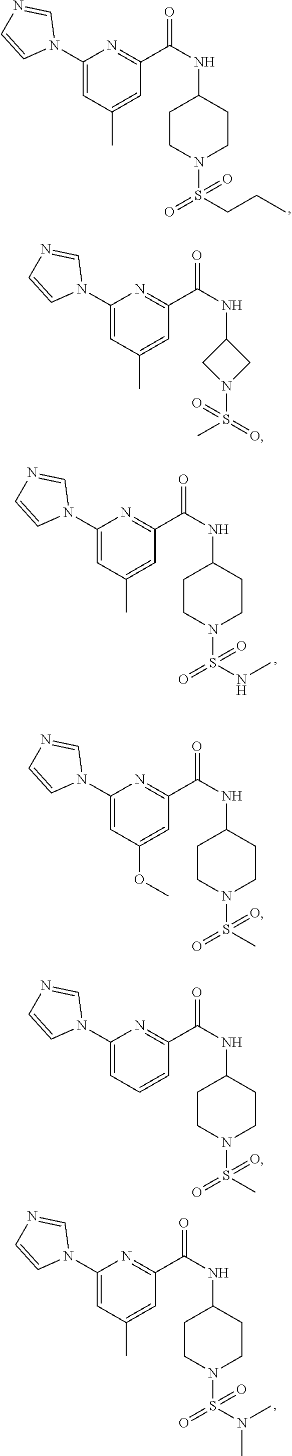 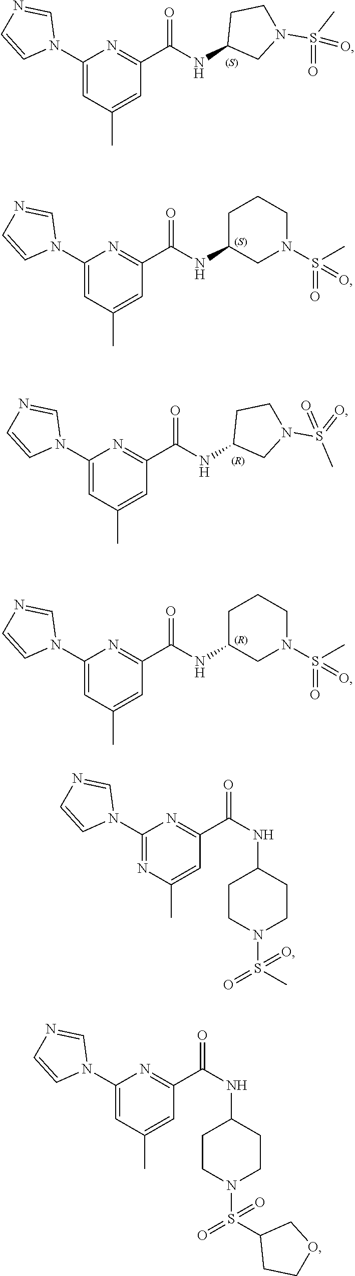 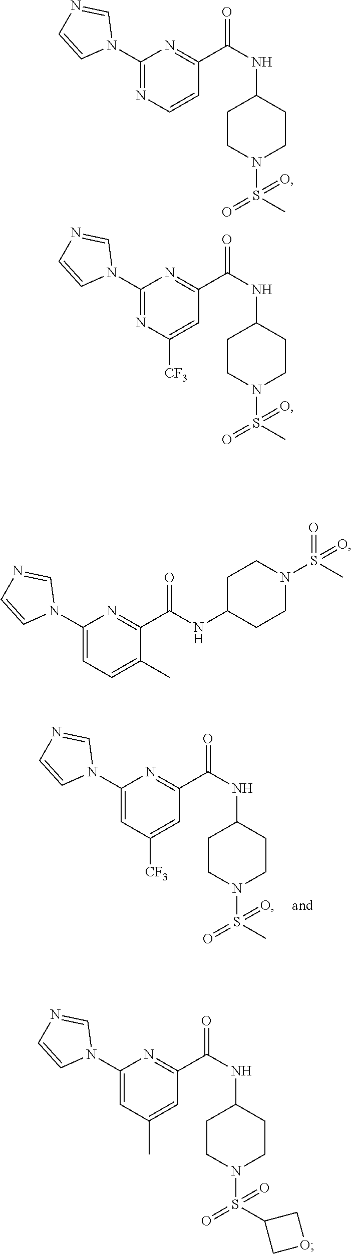
a pharmaceutically acceptable salt of any one thereof.

155. The compound or salt of claim 142, selected from: 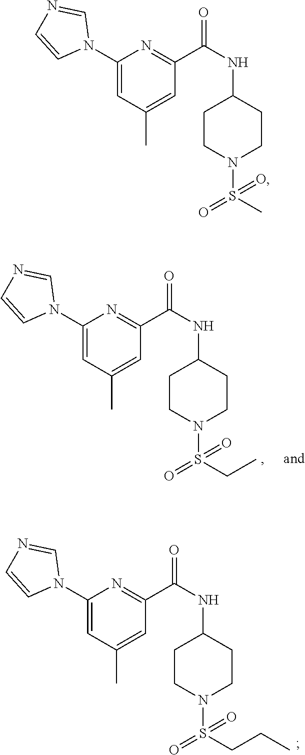 [SMILES: Cc1cc(C(=O)NC2CCN(S(C)(=O)=O)CC2)nc(-n2ccnc2)c1 ; CCS(=O)(=O)N1CCC(NC(=O)c2cc(C)cc(-n3ccnc3)n2)CC1 ; CCCS(=O)(=O)N1CCC(NC(=O)c2cc(C)cc(-n3ccnc3)n2)CC1]
a pharmaceutically acceptable salt of any one thereof.

156. The compound or salt of claim 142, selected from: 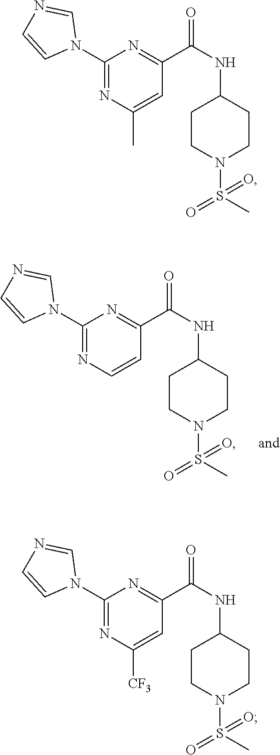 [SMILES: Cc1cc(C(=O)NC2CCN(S(C)(=O)=O)CC2)nc(-n2ccnc2)n1 ; CS(=O)(=O)N1CCC(NC(=O)c2ccnc(-n3ccnc3)n2)CC1 ; CS(=O)(=O)N1CCC(NC(=O)c2cc(C(F)(F)F)nc(-n3ccnc3)n2)CC1]
a pharmaceutically acceptable salt of any one thereof.

157. The compound or salt of claim 142, selected from: 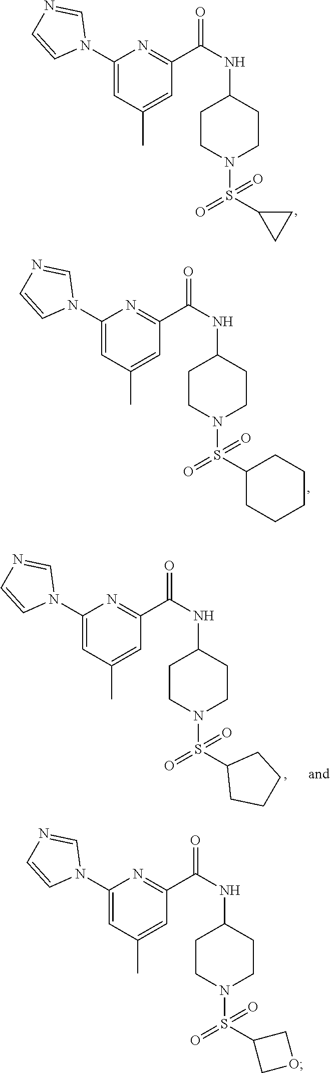 [SMILES: Cc1cc(C(=O)NC2CCN(S(=O)(=O)C3CC3)CC2)nc(-n2ccnc2)c1 ; Cc1cc(C(=O)NC2CCN(S(=O)(=O)C3CCCCC3)CC2)nc(-n2ccnc2)c1 ; Cc1cc(C(=O)NC2CCN(S(=O)(=O)C3CCCC3)CC2)nc(-n2ccnc2)c1 ; Cc1cc(C(=O)NC2CCN(S(=O)(=O)C3COC3)CC2)nc(-n2ccnc2)c1]
or
a pharmaceutically acceptable salt of any one thereof.

158. A pharmaceutical composition comprising a compound or salt of claim 142 and a pharmaceutically acceptable excipient.

159. A method of treating a disease, comprising administering to a subject in need thereof a compound or salt of claim 142 or the pharmaceutical composition of claim 158, wherein the subject would benefit from inhibition of CD38.

160. The method of claim 159, wherein the disease is a selected from neurodegenerative disease, type I diabetes, insulin resistance, Leber's hereditary amaurosis, Parkinson's disease, amyelotrophic lateral sclerosis, chronic lymphocytic leukemia, periodontal disease, psoriasis, UV skin damage, radiation protection, diabetic neuropathy, skin hyperpigmentation, Pellagra, Hartnup disease, Diabetes, Huntington's disease, Bipolar disorder, Schizophrenia, postmenopausal osteoporosis, optic neuropathy, neurocognitive disorders, multiple sclerosis, Alzheimer's disease, steatosis, NASH, hearing loss, dyslipidemia, end stage renal disease, Metabolic Syndrome, obesity, sarcopenic obesity, gout, Irritable Bowel Syndrome, Colitis, COPD, Asthma, cystic fibrosis, pancreatitis, idiopathic pulmonary fibrosis, organ reperfusion injury, stroke, muscular dystrophy, cardiac hypertrophy, CHF, leishmaniasis, tuberculosis, hansen's disease, hypoxic pulmonary vasoconstriction, hypertension, renal clear cell carcinoma, small lung cell carcinoma, exercise intolerance, epilepsy, sleep disorders, ataxia—telangiectasia, rheumatoid arthritis, lupus, alcohol intolerance, hyperphosphatemia, acute lung injury, and ARDS.

161. The method of claim 160, wherein the disease is selected from a neurodegenerative disease, obesity, Metabolic Syndrome, and muscular dystrophy.
